(19) United States
(12) Patent Application Publication
(10) Pub. No.: US 2025/0388965 A1
(43) Pub. Date: Dec. 25, 2025

(54) METHODS AND SYSTEMS FOR PROCESSING CELL-FREE SAMPLES

(71) Applicant: CareDx, Inc., Brisbane, CA (US)

(72) Inventors: Robert WOODWARD, Pleasanton, CA (US); Marica GRSKOVIC, Burlingame, CA (US); James YEE, San Mateo, CA (US); Mitch NELLES, Half Moon Bay, CA (US); David HILLER, Brisbane, CA (US)

(21) Appl. No.: 19/308,103

(22) Filed: Aug. 22, 2025

Related U.S. Application Data
(63) Continuation-in-part of application No. 19/281,431, filed on Jul. 25, 2025.
Application No. 19/281,431 is a continuation of application No. 18/936,839, filed on Nov. 4, 2024, now Pat. No. 12,404,547.
Application No. 18/936,839 is a continuation of application No. 18/455,456, filed on Aug. 24, 2023, now abandoned.
Application No. 18/455,456 is a continuation-in-part of application No. 14/658,061, filed on Mar. 13, 2015, now Pat. No. 11,767,559.
(60) Provisional application No. 61/953,582, filed on Mar. 14, 2014.

(51) Int. Cl. C12Q 1/6876; C12Q 1/6806; C12Q 1/686; C12Q 1/6883; G16B 20/00; G16H 50/30

(52) U.S. Cl. CPC C12Q 1/6876; C12Q 1/6806; C12Q 1/686; C12Q 1/6883; G16B 20/00; C12Q 2600/118; C12Q 2600/156; C12Q 2600/158; G16H 50/30; Y02A 90/10

(57) ABSTRACT

Disclosed herein are methods for processing a cell-free sample of a subject, comprising, providing said cell-free sample of said subject, wherein said cell-free sample comprises a plurality of nucleic acid molecules, subjecting said plurality of nucleic acid molecules to one or more amplification reactions to generate a plurality of complementary deoxyribonucleic acid (cDNA) molecules or derivatives thereof, and sequencing said plurality of cDNA molecules or derivatives thereof. Further disclosed herein are systems, comprising a processor, and a non-transitory computer readable storage medium encoded with a computer program that causes said processor to provide a cell-free sample of a subject, wherein said cell-free sample comprises a plurality of nucleic acid molecules, subject said plurality of nucleic acid molecules to one or more amplification reactions to generate a plurality of complementary deoxyribonucleic acid (cDNA) molecules or derivatives thereof, and sequence said plurality of cDNA molecules or derivatives thereof.

### CROSS-REFERENCE TO RELATED APPLICATIONS

This application is a continuation-in-part of U.S. application Ser. No. 19/281,431, filed Jul. 25, 2025, which is a continuation of U.S. application Ser. No. 18/936,839, filed Nov. 4, 2024, which is a continuation of U.S. application Ser. No. 18/455,456, filed Aug. 24, 2023, now abandoned, which is a continuation-in-part of U.S. application Ser. No. 14/658,061, filed Mar. 13, 2015, now issued as U.S. Pat. No. 11,767,559 on Sep. 26, 2023, which claims the benefit of U.S. Provisional Application No. 61/953,582, filed Mar. 14, 2014.

### FIELD

The present disclosure relates to methods and systems for processing a cell-free sample of a subject and sequencing complementary deoxyribonucleic acid (cDNA) therefrom.

### BACKGROUND

The immune system plays a defensive role in subjects, such as a human individual, but can also cause diseases, disorders, and other undesirable conditions. In the case of medically intended transplantation of non-self (allograft) cells, tissues, or organs into an individual, the recipient's immune system recognizes the allograft to be foreign to the body and activates various mechanisms to reject the allograft. Thus, it is necessary to medically suppress the normal immune system responses to reject the transplant. The medical practice of immunosuppression in transplant recipients has evolved to include a regimen of prophylactic pharmacologic agents, typically beginning with induction therapies to deplete lymphocytes, followed by maintenance drugs intended to inhibit activation or replication of lymphocytes such as corticosteroids, calcineurin inhibitors (such as tacrolimus), and additional inhibitors of lymphocyte replication (such as mycophenolate mofetil). Changing or varying the amount of immunosuppressive drugs administered to a transplant recipient has largely been guided by empirical experience. After transplant, the dosage of immunosuppressant(s) are reduced over time to reduce the incidence and severity of side effects, such as increased risk of infectious diseases, while still avoiding immune rejection of the allograft.

After transplantation, the status of the allograft in the transplant recipient may be monitored for the remainder of his/her lifetime, including assessment function of the allograft and immune-mediated rejection of the allograft. In heart transplantation, for example, surveillance for rejection may include up to 15 scheduled biopsies within the first year of the transplant to pro-vide specimens of the heart muscle for histologic evaluation by a pathologist. Each biopsy procedure is invasive (percutaneous passage of a transvenous catheter into the right ventricle of the heart), stressful, inconvenient, and incumbent of procedural risks for the patient, as well as being expensive. Moreover, the biopsy sampling is extremely localized, so histological abnormalities in any non-biopsied areas of the heart are missed. The grading of biopsies is subjective, and discordance of biopsy findings is common between independent pathologists. In the standard clinical care of transplant recipients, there are a variety of clinical laboratory di-agnostics tests, in addition to periodic biopsies, that provide some information relating to the status of the allograft. For example, the serum trough levels of the calcineurin inhibitor drug are measured to estimate adequacy of intended coverage. Other assays detect the presence of antibodies directed against the allograft. Biopsy is primarily used for surveillance of transplant rejection within the first year, but this invasive method is not well suited or established for guiding individualized immunosuppressive therapy in the longer term (e.g beyond one year after transplant) maintenance care of patients. Non-invasive gene expression methods inform on the status of the immune system by examining the status of genes expressed in immune cells. AlloMap Molecular Expression Testing is an FDA-cleared test available for heart transplant recipients. Tests are in development for monitoring other solid organ transplants.

In addition to existing invasive biopsy methods of monitoring transplant status, there are currently no specific tests with a demonstrated ability to guide individualization (and further minimization) of immunosuppressive drugs for long-term maintenance of a transplant recipient. Data, mostly derived from registry studies, have identified certain clinical risk factors for transplant loss or death such as recipient age, gender, and race, as well as donor features such as cold ischemia, time, and age. However, determining these clinical risk factors does not sup-plant the need for individualized treatment and routine surveillance of transplant recipients.

There exists a need for improved noninvasive methods of diagnosing and monitoring that status of an allograft in a transplant recipient, as well as for methods of determining the need to adjust immunosuppressive therapy being administered to a transplant recipient.

Additionally, cell, tissue, and organ therapies are critically important medical interventions for the treatment of many life-threatening diseases and ailments including organ failure, tissue damage, cancer, immunodeficiency disorders, neurodegeneration, autoimmune diseases, and genetic diseases. One type of cell therapy is allogeneic cell therapy, in which cells are administered to a recipient with a different genotype. Many different types of allogeneic cells including hematopoietic (i.e., blood-forming) stem cells, skeletal muscle stem cells, cardiac stem cells, mesenchymal stem cells, cardiomyocytes, neurons, lymphocytes, macrophages, dendritic cells, and pancreatic islet cells have been successfully used in the treatment of a variety of diseases and conditions, for example, to replace or repair damaged tissues and/or cells, fight cancer, or treat non-malignant diseases. See, for example, Irion et al. (2016) Mol Ther Methods Clin Dev. 4:72-82; Garbern and Lee (2013) Cell Stem Cell 12(6):689-698; Judson and Rossi (2020) NPJ Regen Med. 5:10; Hatzimichael and Tuthill (2010) Stem Cells Cloning 3:105-117; and Anazawa et al. (2019) Ann Gastroenterol Surg. 3(1):34-42.

In some cell therapies, allogeneic cells such as T cells or NK cells are isolated from a healthy donor and administered to a patient. In other cases, allogeneic cells are administered to replace diseased or dead cells in a recipient. For example, chemotherapy or radiation therapy can destroy the bone marrow of some cancer patients. For these patients, hematopoietic cell transplantation (HCT), also known as bone marrow transplantation or stem cell transplantation, may be performed to replace a patient's damaged or lost cells. In other cases, allogeneic cells are specifically altered or engineered to more effectively and/or specifically treat a particular disease or condition. Such is the case in cellular immunotherapies, where immune cells such as lymphocytes, NK cells, or macrophages are genetically engineered and used to target and kill specific cancer cells. For example, T cells can be modified to produce special structures called chimeric antigen receptors (CARs) on their surfaces that are engineered to target specific cancer antigens; when these CAR T cells are administered into a recipient patient, the CAR receptors enable the CAR T cells to latch onto their target cancer antigens to kill the cancerous cells while leaving healthy tissues unharmed.

Currently, autologous CAR T cell therapy (i.e., where T cells are harvested from the patient, genetically engineered to express CARs, then administered back into the patient) is FDA approved as the standard of care for some forms of aggressive, refractory non-Hodgkin's lymphoma, and for patients with relapsed or refractory acute lymphoblastic leukemia; however, there are several limitations that can make it difficult for patients to access this therapy. For example, the CAR T cells must be made from scratch for each individual patient, resulting in a labor-intensive and costly therapy. In addition, it may not be possible to harvest enough cells from the patient for treatment, or cells may be unsuitable for use in treatment, for example, if the patient's T cells are cancerous. Also, the patient's health may deteriorate or the patient may die before the CAR T cells can be genetically engineered and administered. Allogeneic CAR T cells derived from healthy donors overcome many of these issues, as they can be produced en masse and given to patients immediately. Currently, there are numerous ongoing clinical trials investigating various allogeneic CAR T cell therapies (Aftab, et al. (2020) Advances in Cell and Gene Therapy).

Allogeneic cell therapies could be very effective in treating certain diseases and conditions; however, efficacy requires engraftment, expansion, and/or persistence of the allogeneic cells, where the allogeneic cells are able to survive and/or proliferate in the recipient. Unfortunately, allogeneic cells are frequently recognized as foreign agents by the recipient's immune response. This can trigger an immune response resulting in allogeneic cell rejection, where the allogeneic cells are thought to die and become less numerous over time. Allogeneic cells may also fail to survive in the recipient for reasons other than a host immune response. Eventually, the allogeneic cells can dip below a therapeutically effective threshold, rendering the allogeneic cells ineffective. In this case, there is usually a need to readminister allogeneic cells and/or adjust other therapies to promote allogeneic cell persistence.

In addition, significant side effects associated with allogeneic cell therapies, such as CAR T cell therapy, exist. For example, possible side effects from CAR T cell therapy include graft vs. host disease; cytokine release syndrome, where CAR T cells initiate a massive release of cytokines, triggering an inflammatory condition known as cytokine-release syndrome (CRS); and CAR T related encephalopathy syndrome (CRES), where patients experience neurologic difficulties, confusion, stupor, and/or difficulty understanding language and speech.

Cell therapies are critically important treatment options for many diseases and conditions; however significant risks of rejection and serious side effects associated with allogeneic cell therapy can negatively impact patient health if left unchecked. Methods of monitoring cell engraftment, expansion, persistence, rejection, and disease relapse can help physicians make treatment decisions for their patients, such as when to administer additional allogeneic cells, adjust immunosuppressive therapy, or otherwise adjust therapy to treat allogeneic cell exhaustion, lack of allogeneic cell persistence, allogeneic cell rejection, or side effects of allogeneic cell administration. The ability to quickly and easily monitor therapy is extremely valuable, as early detection of allogeneic cell engraftment, expansion, contraction, persistence, rejection, and/or disease relapse allows for early treatment and better therapeutic outcomes for recipients of allogeneic cell therapy. Monitoring allogeneic cell status and therapeutic effectiveness during allogeneic cellular therapies might also provide better insight into the mechanisms underlying clinical efficacy of allogeneic cell therapy, which is pivotal for further development of immune therapies to treat cancer.

Though monitoring of allogeneic cell therapy is extremely important, few methods of monitoring exist. Flow cytometry and immunohistochemistry of patient samples are sometimes used to monitor allogeneic cells; for example, allogeneic cell levels, health, phenotype, and effectiveness may be assessed by profiling various cell surface antigens. However, these techniques are analytically challenging, labor intensive, variable among laboratories, and have suboptimal sensitivity and/or specificity. In addition, these methods require prior knowledge of the surface antigens of interest. Another way of monitoring allogeneic cell therapy is through the analysis of the levels of allogeneic and recipient microsatellites and minisatellites (i.e., tandemly repeated blocks of non-coding DNA) in patient samples, which tend to differ between individuals. However, this method is only semi-quantitative and moderately sensitive (i.e., can detect a minimum minor allele level as low as about 1-5%). One more way of monitoring allogeneic cell therapy is through quantitative or digital PCR to detect particular biomarkers or tags specific to the allogeneic cells. These methods have a higher sensitivity than flow cytometry, immunohistochemistry, and microsatellite/minisatellite profiling; however they too require complex methodological optimization and prior knowledge of the biomarkers or tags of interest (e.g., by sequencing allogeneic and/or recipient cells). These methods also use a significant amount of DNA material, and are not linear across the full range 0-100%.

Thus, there exists a need for improved methods of monitoring allogeneic cell status and the therapeutic effectiveness of allogeneic cells that can better inform treatment decisions related to allogeneic cell therapy. In particular, there exists a need for methods that can be applied universally to allogeneic cells, regardless of whether they have been genetically modified or not. There also exists a need for improved methods of treating allogeneic cell rejection and administering allogeneic cells that are informed by allogeneic cell status and/or therapeutic effectiveness.

### BRIEF SUMMARY

In one aspect, the present disclosure relates to methods of monitoring immunosuppressive therapy in a subject, the method including: a) providing cell-free DNA from a sample obtained from a subject who is the recipient of an organ transplant from a donor, b) sequencing a panel of single nucleotide polymorphisms (SNPs) from the cell-free DNA, where the panel of SNPs is suitable for differentiating between donor-derived cell-free DNA and recipient-derived cell-free DNA, c) assaying variance in SNP allele distribution patterns in the panel as compared to expected homozygous or heterozygous distribution patterns to determine the level of donor-derived cell-free DNA, and d) diagnosing the status of the transplanted organ in the subject, where a change in levels or variance of the donor-derived cell-free DNA over a time interval is indicative of transplanted organ status and a basis for adjusting immunosuppressive therapy.

In another aspect, the present disclosure relates to methods of adjusting an immunosuppressive therapy in a subject, the method including: a) providing cell-free DNA from a sample obtained from a subject who is the recipient of an organ transplant from a donor, b) sequencing a panel of single nucleotide polymorphisms (SNPs) from the cell-free DNA, where the panel of SNPs is suitable for differentiating between donor-derived cell-free DNA and recipient-derived cell-free DNA, c) assaying variance in SNP allele distribution patterns in the panel as compared to expected homozygous or heterozygous distribution patterns to determine the level of donor-derived cell-free DNA, d) diagnosing the status of the transplanted organ in the subject, where a change in levels or variance of the donor-derived cell-free DNA over a time interval is indicative of transplanted organ status, e) adjusting immunosuppressive therapy being administered to the subject.

In some embodiments that may be combined with any of the preceding embodiments, an increase in the levels or variance of the donor-derived cell-free DNA over the time interval is indicative of transplant rejection, a need for adjusting immunosuppressive therapy, and/or a need for further investigation of the transplanted organ status. In some embodiments that may be combined with any of the preceding embodiments, a decrease in the levels or variance of the donor-derived cell-free DNA over the time interval is indicative of transplant tolerance, a need for adjusting immunosuppressive therapy, and/or a need for further investigation of the transplanted organ status. In some embodiments that may be combined with any of the preceding embodiments, no change in the levels or variance of the donor-derived cell-free DNA over the time interval is indicative of stable transplant rejection status and/or opportunity for adjusting immunosuppressive therapy. In some embodiments that may be combined with any of the preceding embodiments, immunosuppressive therapy being administered to the subject is increased. In some embodiments that may be combined with any of the preceding embodiments, immunosuppressive therapy being administered to the subject is decreased. In some embodiments that may be combined with any of the preceding embodiments, immunosuppressive therapy being administered to the subject is maintained. In some embodiments that may be combined with any of the preceding embodiments, the organ transplant is a kidney transplant. In some embodiments that may be combined with any of the preceding embodiments, the organ transplant is a heart transplant. In some embodiments that may be combined with any of the preceding embodiments, the sample is a plasma sample. In some embodiments that may be combined with any of the preceding embodiments, the panel of SNPs includes at least 20 independent SNPs. In some embodiments that may be combined with any of the preceding embodiments, the panel of SNPs includes independent SNPs selected from rs1004357, rs10092491, rs1019029, rs1027895, rs10488710, rs10500617, rs1058083, rs10768550, rs10773760, rs10776839, rs1109037, rs12480506, rs1294331, rs12997453, rs13134862, rs13182883, rs13218440, rs1336071, rs1358856, rs1410059, rs1478829, rs1490413, rs1498553, rs1523537, rs1554472, rs159606, rs1736442, rs1821380, rs1872575, rs2046361, rs2073383, rs214955, rs2175957, rs221956, rs2255301, rs2269355, rs2270529, rs2272998, rs2291395, rs2292972, rs2342747, rs2399332, rs2503107, rs2567608, rs279844, rs2811231, rs2833736, rs2920816, rs315791, rs321198, rs338882, rs3744163, rs3780962, rs4288409, rs430046, rs4364205, rs445251, rs4530059, rs4606077, rs464663, rs4789798, rs4796362, rs4847034, rs521861, rs560681, rs5746846, rs576261, rs590162, rs6444724, rs6591147, rs6811238, rs689512, rs6955448, rs7041158, rs7205345, rs722290, rs7229946, rs740598, rs7520386, rs7704770, rs8070085, rs8078417, rs891700, rs901398, rs9546538, rs9606186, rs985492, rs9866013, rs987640, rs9905977, rs993934, and rs9951171. In some embodiments that may be combined with any of the preceding embodiments, sequencing the panel of SNPs is performed using a multiplex sequencing platform. In some embodiments that may be combined with any of the preceding embodiments, the time interval is about 12-14 months after the transplant from the donor to the recipient subject occurred. In some embodiments that may be combined with any of the preceding embodiments, the method further includes testing for viral load. In some embodiments, the testing includes determining the presence of a virus selected from CMV, EBV, anellovirus, and BKV. In some embodiments that may be combined with any of the preceding embodiments, the method further includes conducting one or more gene expression profiling assays. In some embodiments, the gene expression profiling assay is an AlloMap test.

In one aspect, the present disclosure relates to methods of monitoring the status of a transplanted organ in a subject, the method including: a) providing cell-free DNA from a sample obtained from a subject who is the recipient of an organ transplant from a donor, b) sequencing a panel of single nucleotide polymorphisms (SNPs) from the cell-free DNA, where the panel of SNPs is suitable for differentiating between donor-derived cell-free DNA and recipient-derived cell-free DNA, c) assaying variance in SNP allele distribution patterns in the panel as compared to expected homozygous or heterozygous distribution patterns to determine the level of donor-derived cell-free DNA, and d) diagnosing the status of the transplanted organ in the subject, where a change in levels or variance of the donor-derived cell-free DNA over a time interval is indicative of the status of the transplanted organ.

In another aspect, the present disclosure relates to methods of monitoring immunosuppressive therapy in a subject, the method including: a) providing cell-free DNA from a sample obtained from a subject who is the recipient of an organ transplant from a donor, b) sequencing a panel of single nucleotide polymorphisms (SNPs) from the cell-free DNA, where the panel of SNPs is suitable for differentiating between donor-derived cell-free DNA and recipient-derived cell-free DNA, c) assaying variance in SNP allele distribution patterns in the panel as compared to expected homozygous or heterozygous distribution patterns to determine the level of donor-derived cell-free DNA, and d) diagnosing the status of the transplanted organ in the subject, where a change in levels or variance of the donor-derived cell-free DNA over a time interval is indicative of transplanted organ status and a basis for adjusting immunosuppressive therapy.

In another aspect, the present disclosure relates to methods of adjusting an immunosuppressive therapy in a subject, the method including: a) providing cell-free DNA from a sample obtained from a subject who is the recipient of an organ transplant from a donor, b) sequencing a panel of single nucleotide polymorphisms (SNPs) from the cell-free DNA, where the panel of SNPs is suitable for differentiating between donor-derived cell-free DNA and recipient-derived cell-free DNA, c) assaying variance in SNP allele distribution patterns in the panel as compared to expected homozygous or heterozygous distribution patterns to determine the level of donor-derived cell-free DNA, d) diagnosing the status of the transplanted organ in the subject, where a change in levels or variance of the donor-derived cell-free DNA over a time interval is indicative of transplanted organ status, and e) adjusting immunosuppressive therapy being administered to the subject.

In some embodiments that may be combined with any of the preceding embodiments, an increase in the levels or variance of the donor-derived cell-free DNA over the time interval is indicative of transplant rejection, a need for adjusting immunosuppressive therapy, and/or a need for further investigation of the transplanted organ status. In some embodiments that may be combined with any of the preceding embodiments, a decrease in the levels or variance of the donor-derived cell-free DNA over the time interval is indicative of transplant tolerance, a need for adjusting immunosuppressive therapy, and/or a need for further investigation of the transplanted organ status. In some embodiments that may be combined with any of the preceding embodiments, no change in the levels or variance of the donor-derived cell-free DNA over the time interval is indicative of stable transplant rejection status and/or opportunity for adjusting immunosuppressive therapy. In some embodiments that may be combined with any of the preceding embodiments, immunosuppressive therapy being administered to the subject is increased. In some embodiments that may be combined with any of the preceding embodiments, immunosuppressive therapy being administered to the subject is decreased. In some embodiments that may be combined with any of the preceding embodiments, immunosuppressive therapy being administered to the subject is maintained. In some embodiments that may be combined with any of the preceding embodiments, the organ transplant is a kidney transplant. In some embodiments that may be combined with any of the preceding embodiments, the organ transplant is a heart transplant. In some embodiments that may be combined with any of the preceding embodiments, the organ transplant is selected from a liver transplant, a lung transplant, and a pancreas transplant. In some embodiments that may be combined with any of the preceding embodiments, the sample is a plasma sample. In some embodiments that may be combined with any of the preceding embodiments, the panel of SNPs includes independent SNPs selected from rs1004357, rs10092491, rs1019029, rs1027895, rs10488710, rs10500617, rs1058083, rs10768550, rs10773760, rs10776839, rs1109037, rs12480506, rs1294331, rs12997453, rs13134862, rs13182883, rs13218440, rs1336071, rs1358856, rs1410059, rs1478829, rs1490413, rs1498553, rs1523537, rs1554472, rs159606, rs1736442, rs1821380, rs1872575, rs2046361, rs2073383, rs214955, rs2175957, rs221956, rs2255301, rs2269355, rs2270529, rs2272998, rs2291395, rs2292972, rs2342747, rs2399332, rs2503107, rs2567608, rs279844, rs2811231, rs2833736, rs2920816, rs315791, rs321198, rs338882, rs3744163, rs3780962, rs4288409, rs430046, rs4364205, rs445251, rs4530059, rs4606077, rs464663, rs4789798, rs4796362, rs4847034, rs521861, rs560681, rs5746846, rs576261, rs590162, rs6444724, rs6591147, rs6811238, rs689512, rs6955448, rs7041158, rs7205345, rs722290, rs7229946, rs740598, rs7520386, rs7704770, rs8070085, rs8078417, rs891700, rs901398, rs9546538, rs9606186, rs985492, rs9866013, rs987640, rs9905977, rs993934, and rs9951171. In some embodiments that may be combined with any of the preceding embodiments, the panel of SNPs includes SNPs that have an overall population minor allele frequency of >0.4, a target population minor allele frequency of >0.4, the lowest polymerase error rate of the 6 potential allele transitions or transversions, and the genomic distance between each independent SNP is >500 kb. In some embodiments that may be combined with any of the preceding embodiments, the panel of SNPs includes independent SNPs selected from rs10488710, rs279844, rs1048290, rs1049379, rs1051614, rs1052637, rs1055851, rs1056033, rs1056149, rs1064074, rs1078004, rs10831567, rs6811238, rs11106, rs11210490, rs1126899, rs1127472, rs1127893, rs1130857, rs1049544, rs11547806, rs12237048, rs430046, rs12508837, rs12529, rs12717, rs13184586, rs13295990, rs13428, rs13436, rs1374570, rs14080, rs1411271, rs576261, rs14155, rs1151687, rs1565933, rs1600, rs1678690, rs1881421, rs1897820, rs1898882, rs2056844, rs20575, rs10092491, rs2070426, rs2071888, rs2075322, rs2180314, rs2185798, rs2227910, rs2228560, rs2229571, rs2229627, rs2245285, rs2342747, rs2248490, rs2253592, rs2254357, rs2275047, rs2279665, rs2279776, rs2281098, rs2287813, rs4364205, rs2289751, rs2289818, rs2292830, rs2294092, rs2295005, rs2296545, rs2297236, rs2302443, rs2306049, rs1022478, rs445251, rs230898, rs231235, rs2342767, rs236152, rs2362450, rs2384571, rs2455230, rs246703, rs2480345, rs248385, rs2498982, rs2505232, rs2509943, rs2519123, rs2523072, rs2571028, rs2657167, rs28686812, rs2946994, rs1294331, rs10419826, rs3088241, rs3110623, rs3173615, rs3190321, rs3205187, rs344141, rs35596415, rs362124, rs36657, rs1872575, rs159606, rs3731877, rs3734311, rs3735615, rs3740199, rs3748930, rs3751066, rs3790993, rs3802265, rs3803763, rs1004357, rs3803798, rs3809972, rs3810483, rs3812571, rs3813609, rs3814182, rs3816800, rs3826709, rs3829655, rs3951216, rs1019029, rs408600, rs41317515, rs436278, rs448012, rs475002, rs4845480, rs4849167, rs4865615, rs1027895, rs4890012, rs492594, rs4940019, rs4971514, rs523104, rs528557, rs545500, rs561930, rs57010808, rs57285449, rs10500617, rs6061243, rs609521, rs62490396, rs625223, rs638405, rs6459166, rs648802, rs6510057, rs6764714, rs10768550, rs6790129, rs6794, rs6807362, rs6838248, rs713598, rs7161563, rs726009, rs7289, rs7301328, rs7332388, rs10773760, rs743616, rs743852, rs745142, rs7451713, rs7526132, rs7543016, rs7601771, rs7785899, rs7825, rs8009219, rs10776839, rs8025851, rs8058696, rs8076632, rs8097, rs8103906, rs874881, rs9262, rs9289122, rs936019, rs9393728, rs1109037, rs977070, rs9865242, rs12480506, rs560681, rs12997453, rs13134862, rs13218440, rs1358856, rs1410059, rs1478829, rs1498553, rs1523537, rs4606077, rs1554472, rs1736442, rs1821380, rs2046361, rs214955, rs2175957, rs2255301, rs2269355, rs2270529, rs2272998, rs2291395, rs2292972, rs2399332, rs2503107, rs2567608, rs2811231, rs2833736, rs315791, rs321198, rs6955448, rs338882, rs3780962, rs4288409, rs4530059, rs464663, rs4789798, rs4796362, rs4847034, rs521861, rs1058083, rs5746846, rs590162, rs6444724, rs6591147, rs689512, rs7205345, rs722290, rs740598, rs7520386, rs221956, rs7704770, rs8070085, rs8078417, rs891700, rs901398, rs9546538, rs9606186, rs985492, rs9866013, rs987640, rs13182883, rs9905977, rs993934, rs9951171, rs10274334, rs10421285, rs1043413, rs1044010, rs1045248, rs1045644, and rs1047979. In some embodiments, the SNP panel includes about 195 to about 200, about 200 to about 205, about 210 to about 215, about 215 to about 220, about 220 to about 225, about 225 to about 230, about 230 to about 235, about 235 to about 240, about 240 to about 245, about 245 to about 250, about 250 to about 255, about 255 to about 260, about 260 to about 265, or about 260 to about 266 of the independent SNPs. In some embodiments that may be combined with any of the preceding embodiments, the SNP panel includes rs10488710, rs279844, rs1048290, rs1049379, rs1051614, rs1052637, rs1055851, rs1056033, rs1056149, rs1064074, rs1078004, rs10831567, rs6811238, rs11106, rs11210490, rs1126899, rs1127472, rs1127893, rs1130857, rs1049544, rs11547806, rs12237048, rs430046, rs12508837, rs12529, rs12717, rs13184586, rs13295990, rs13428, rs13436, rs1374570, rs14080, rs1411271, rs576261, rs14155, rs1151687, rs1565933, rs1600, rs1678690, rs1881421, rs1897820, rs1898882, rs2056844, rs20575, rs10092491, rs2070426, rs2071888, rs2075322, rs2180314, rs2185798, rs2227910, rs2228560, rs2229571, rs2229627, rs2245285, rs2342747, rs2248490, rs2253592, rs2254357, rs2275047, rs2279665, rs2279776, rs2281098, rs2287813, rs4364205, rs2289751, rs2289818, rs2292830, rs2294092, rs2295005, rs2296545, rs2297236, rs2302443, rs2306049, rs1022478, rs445251, rs230898, rs231235, rs2342767, rs236152, rs2362450, rs2384571, rs2455230, rs246703, rs2480345, rs248385, rs2498982, rs2505232, rs2509943, rs2519123, rs2523072, rs2571028, rs2657167, rs28686812, rs2946994, rs1294331, rs10419826, rs3088241, rs3110623, rs3173615, rs3190321, rs3205187, rs344141, rs35596415, rs362124, rs36657, rs1872575, rs159606, rs3731877, rs3734311, rs3735615, rs3740199, rs3748930, rs3751066, rs3790993, rs3802265, rs3803763, rs1004357, rs3803798, rs3809972, rs3810483, rs3812571, rs3813609, rs3814182, rs3816800, rs3826709, rs3829655, rs3951216, rs1019029, rs408600, rs41317515, rs436278, rs448012, rs475002, rs4845480, rs4849167, rs4865615, rs1027895, rs4890012, rs492594, rs4940019, rs4971514, rs523104, rs528557, rs545500, rs561930, rs57010808, rs57285449, rs10500617, rs6061243, rs609521, rs62490396, rs625223, rs638405, rs6459166, rs648802, rs6510057, rs6764714, rs10768550, rs6790129, rs6794, rs6807362, rs6838248, rs713598, rs7161563, rs726009, rs7289, rs7301328, rs7332388, rs10773760, rs743616, rs743852, rs745142, rs7451713, rs7526132, rs7543016, rs7601771, rs7785899, rs7825, rs8009219, rs10776839, rs8025851, rs8058696, rs8076632, rs8097, rs8103906, rs874881, rs9262, rs9289122, rs936019, rs9393728, rs1109037, rs977070, rs9865242, rs12480506, rs560681, rs12997453, rs13134862, rs13218440, rs1358856, rs1410059, rs1478829, rs1498553, rs1523537, rs4606077, rs1554472, rs1736442, rs1821380, rs2046361, rs214955, rs2175957, rs2255301, rs2269355, rs2270529, rs2272998, rs2291395, rs2292972, rs2399332, rs2503107, rs2567608, rs2811231, rs2833736, rs315791, rs321198, rs6955448, rs338882, rs3780962, rs4288409, rs4530059, rs464663, rs4789798, rs4796362, rs4847034, rs521861, rs1058083, rs5746846, rs590162, rs6444724, rs6591147, rs689512, rs7205345, rs722290, rs740598, rs7520386, rs221956, rs7704770, rs8070085, rs8078417, rs891700, rs901398, rs9546538, rs9606186, rs985492, rs9866013, rs987640, rs13182883, rs9905977, rs993934, rs9951171, rs10274334, rs10421285, rs1043413, rs1044010, rs1045248, rs1045644, and rs1047979. In some embodiments that may be combined with any of the preceding embodiments, sequencing the panel of SNPs is performed using a multiplex sequencing platform. In some embodiments that may be combined with any of the preceding embodiments, the level of donor-derived cell-free DNA in the sample is determined without using genotype information. In some embodiments that may be combined with any of the preceding embodiments, the method further includes testing for the presence of an infectious agent. In some embodiments that may be combined with any of the preceding embodiments, the infectious agent is selected from viruses, bacteria, fungi, and parasites. In some embodiments, the viruses are selected from Cytomegalovirus, Epstein-Barr virus, Anelloviridae, and BK virus. In some embodiments that may be combined with any of the preceding embodiments, the method further includes conducting one or more gene expression profiling assays. In some embodiments, a combination score is calculated based on the results of the level of donor-derived cell-free DNA and the results of the gene expression profiling assay. In some embodiments that may be combined with any of the preceding embodiments, the gene expression profiling assay is an AlloMap test.

In one aspect, the present disclosure provides a method of administering allogeneic cells to a recipient and adjusting treatment or monitoring of the recipient, the method comprising: a) administering allogeneic cells to the recipient; b) providing cell DNA from a sample obtained from the recipient; c) sequencing a panel of single nucleotide polymorphisms (SNPs) from the cell DNA, wherein the panel of SNPs is suitable for differentiating between allogeneic cell DNA and recipient-derived cell DNA; d) assaying differences in SNP allele distribution patterns in the panel as compared to expected homozygous or heterozygous distribution patterns to determine the level of allogeneic cell DNA; wherein a level of allogeneic cell DNA above a therapeutically effective threshold and/or increasing or stable over a time interval indicates a status of engraftment, expansion and/or persistence of the allogeneic cells, and a level of allogeneic cell DNA below a therapeutically effective threshold and/or decreasing over a time interval indicates a status of exhaustion, contraction, loss of persistence, allogeneic cell rejection, and/or graft vs. host disease; and e) adjusting treatment or monitoring of the recipient of the allogeneic cells based on the status of the allogeneic cells.

In some embodiments, the level of allogeneic cell DNA is above a therapeutically effective threshold and/or increasing or stable over a time interval, thereby indicating a status of engraftment, expansion, and/or persistence of the allogeneic cells; and wherein adjusting the treatment of the recipient of the allogeneic cells comprises: administering allogeneic cells that are different than those administered in step a), administering a reduced dose of the allogeneic cells, administering doses of the allogeneic cells less frequently, discontinuing administration of the allogeneic cells, or combinations thereof.

In some embodiments, which may be combined with any of the preceding embodiments, the level of allogeneic cell DNA is above a therapeutically effective threshold and/or increasing or stable over a time interval, thereby indicating a status of engraftment, expansion, and/or persistence of the allogeneic cells; and wherein adjusting the treatment of the recipient of the allogeneic cells comprises: reducing immunosuppressive therapy, and/or discontinuing administration of immunosuppressive therapy.

In certain embodiments, allogeneic cell rejection is due to host vs. graft disease.

In certain embodiments, the level of allogeneic cell DNA is below a therapeutically effective threshold and/or decreasing over a time interval, thereby indicating a status of exhaustion, contraction, loss of persistence, allogeneic cell rejection, and/or graft vs. host disease; and wherein adjusting treatment of the recipient of the allogeneic cells comprises: continuing administration of allogeneic cells that are the same as those administered in step a), administering allogeneic cells that are different than those administered in step a), administering an increased dose of the allogeneic cells, administering doses of the allogeneic cells more frequently, or combinations thereof.

In certain embodiments, the level of allogeneic cell DNA is below a therapeutically effective threshold and/or decreasing over a time interval, thereby indicating a status of exhaustion, contraction, loss of persistence, allogeneic cell rejection, and/or graft vs. host disease; and wherein adjusting treatment of the recipient of the allogeneic cells comprises: initiating immunosuppressive therapy, and/or adjusting immunosuppressive therapy.

In some embodiments, which may be combined with any of the preceding embodiments, the sample is obtained twice a week in the first three weeks after step a), once a week for the first three months after step a), once a month for the first year after step a), and four times a year after the first year after step a), for at least one year. In some embodiments, which may be combined with any of the preceding embodiments, the sample is obtained at least once a week in the first three weeks after step a), at least once a week for the first three months after step a), at least once a month for the first year after step a), or several times a year after the first year after step a), for at least one year.

In another aspect, the present disclosure provides a method of determining the status of allogeneic cells in a recipient of allogeneic cells, the method comprising: a) optionally administering the allogeneic cells to the recipient; b) providing cell DNA from a sample obtained from the recipient; c) sequencing a panel of single nucleotide polymorphisms (SNPs) from the cell DNA, wherein the panel of SNPs is suitable for differentiating between allogeneic cell DNA and recipient-derived cell DNA; and d) assaying differences in SNP allele distribution patterns in the panel as compared to expected homozygous or heterozygous distribution patterns to determine the level of allogeneic cell DNA; wherein a level of allogeneic cell DNA above a therapeutically effective threshold and/or increasing or stable over a time interval indicates a status of engraftment, expansion and/or persistence of the allogeneic cells, and a level of allogeneic cell DNA below a therapeutically effective threshold and/or decreasing over a time interval indicates a status of exhaustion, contraction, loss of persistence, allogeneic cell rejection, and/or graft vs. host disease.

In another aspect, the present disclosure provides a method of treating allogeneic cell rejection in a recipient, the method comprising: a) providing cell DNA from a sample obtained from the recipient of allogeneic cells; b) sequencing a panel of single nucleotide polymorphisms (SNPs) from the cell DNA, wherein the panel of SNPs is suitable for differentiating between allogeneic cell DNA and recipient-derived cell DNA; c) assaying differences in SNP allele distribution patterns in the panel as compared to expected homozygous or heterozygous distribution patterns to determine the level of allogeneic cell DNA; d) diagnosing the recipient as experiencing allogeneic cell rejection, wherein a level of allogeneic cell DNA below a therapeutically effective threshold and/or decreasing over a time interval indicates allogeneic cell rejection; and e) administering an immunosuppressive therapy or adjusting ongoing immunosuppressive therapy to the recipient diagnosed as exhibiting allogeneic cell rejection.

In another aspect, the present disclosure provides a method of monitoring for relapse of a hematologic cancer in a recipient, the method comprising: a) providing cell DNA from a sample obtained from the recipient of allogeneic cells; b) sequencing a panel of single nucleotide polymorphisms (SNPs) from the cell DNA, wherein the panel of SNPs is suitable for differentiating between allogeneic cell DNA and recipient-derived cell DNA; c) assaying differences in SNP allele distribution patterns in the panel as compared to expected homozygous or heterozygous distribution patterns to determine the level of allogeneic cell DNA; and d) monitoring for relapse based on the level of allogeneic cell DNA. In some embodiments, a relapse is indicated by a decrease of the level of allogeneic cell DNA overtime in comparison to a prior determination of the level of allogeneic cell DNA. In some embodiments, if there appears to be relapse of the hematologic cancer, treatment of the recipient with allogeneic cells is re-initiated, allogeneic cells are administered that are different than those originally administered, chemotherapy is administered, a targeted anti-leukemia therapy is administered, immunotherapy is administered, palliative care is administered, the recipient is monitored more frequently, and/or the relapse is confirmed using other measures. In some embodiments, in case of a relapse the method further comprises re-initiating treatment of the recipient with allogeneic cells, administering allogeneic cells that are different than those originally administered, administering chemotherapy, administering a targeted anti-leukemia therapy, administering immunotherapy, administering palliative care, more frequent monitoring of the recipient, and/or confirming the relapse using other measures.

In another aspect, the present disclosure provides a method of measuring the level of chimerism in a sample, the method comprising: a) providing cell DNA from a sample obtained from a recipient of allogeneic cells; b) sequencing a panel of single nucleotide polymorphisms (SNPs) from the cell DNA, wherein the panel of SNPs is suitable for differentiating between allogeneic cell DNA and recipient-derived cell DNA; c) assaying differences in SNP allele distribution patterns in the panel as compared to expected homozygous or heterozygous distribution patterns to determine the level of allogeneic cell DNA; and d) determining the level of chimerism in the sample.

In another aspect, the present disclosure provides a method of measuring a cellular kinetic parameter of allogeneic cells in a recipient, the method comprising: a) providing cell DNA from a series of samples obtained from the recipient of allogeneic cells over a period of time; b) sequencing a panel of single nucleotide polymorphisms (SNPs) from the cell DNA, wherein the panel of SNPs is suitable for differentiating between allogeneic cell DNA and recipient-derived cell DNA; and c) assaying differences in SNP allele distribution patterns in the panel as compared to expected homozygous or heterozygous distribution patterns to determine the level of allogeneic cell DNA in the series of samples; thereby measuring the cellular kinetic parameter of allogeneic cells in the recipient.

In certain embodiments, the cellular kinetic parameter is selected from the group consisting of Cₘₐₓ, tₘₐₓ, AUC, rate of contraction, rate of engraftment, and a measurement of persistence.

In another aspect, the present disclosure provides a method of identifying allogeneic cells in a recipient, the method comprising: a) providing cell DNA from a sample obtained from the recipient of allogeneic cells; b) sequencing a panel of single nucleotide polymorphisms (SNPs) from the cell DNA, wherein the panel of SNPs is suitable for differentiating between allogeneic cell DNA and recipient-derived cell DNA; and c) assaying differences in SNP allele distribution patterns in the panel as compared to expected homozygous or heterozygous distribution patterns to identify the allogeneic cell DNA; thereby identifying the allogeneic cells in the recipient. In some embodiments, identifying the allogeneic cells in the recipient is used to detect the presence of allogeneic cells in a recipient.

In another aspect, the present disclosure provides a method of predicting recipient responsiveness to allogeneic cell administration, the method comprising: a) providing cell DNA from a sample obtained from the recipient of allogeneic cells; b) sequencing a panel of single nucleotide polymorphisms (SNPs) from the cell DNA, wherein the panel of SNPs is suitable for differentiating between allogeneic cell DNA and recipient-derived cell DNA; and c) assaying differences in SNP allele distribution patterns in the panel as compared to expected homozygous or heterozygous distribution patterns to determine the level of allogeneic cell DNA; wherein a level of allogeneic cell DNA above a therapeutically effective threshold and/or increasing or stable over a time interval indicates that it is more likely that the recipient will respond to the allogeneic cells, and a level of allogeneic cell DNA below a therapeutically effective threshold and/or decreasing over a time interval indicates that it is less likely that the recipient will respond to the allogeneic cells; thereby predicting recipient responsiveness to allogeneic cell therapy.

In another aspect, the present disclosure provides a method of identifying recipients at a higher risk for a side effect associated with allogeneic cell administration, the method comprising: a) providing cell DNA from a sample obtained from a recipient of allogeneic cells; b) sequencing a panel of single nucleotide polymorphisms (SNPs) from the cell DNA, wherein the panel of SNPs is suitable for differentiating between allogeneic cell DNA and recipient-derived cell DNA; and c) assaying differences in SNP allele distribution patterns in the panel as compared to expected homozygous or heterozygous distribution patterns to determine the level of allogeneic cell DNA; wherein a level of allogeneic cell DNA above a safety threshold indicates that the recipient is at a higher risk of a side effect associated with allogeneic cell administration, and a level of allogeneic cell DNA below a safety threshold indicates that the recipient is at a lower risk of a side effect associated with allogeneic cell administration; thereby identifying recipients at a higher risk for a side effect associated with allogeneic cell administration.

In some embodiments, which may be combined with any of the preceding aspects or embodiments, individual genotyping of the allogeneic cells and the recipient to determine which allele of the SNP belongs to the allogeneic cells and the recipient is not performed.

In some embodiments, which may be combined with any of the preceding aspects or embodiments, the sample is a whole blood, plasma, serum, or peripheral blood mononuclear cell (PBMC) sample.

In some embodiments, which may be combined with any of the preceding aspects or embodiments, the level of allogeneic cell DNA is expressed as the percentage of allogeneic cells or the area under the curve (AUC) of the percentage of allogeneic cells over a time interval.

In some embodiments, which may be combined with any of the preceding aspects or embodiments, the allogeneic cells are selected from the group consisting of hematopoietic stem cells, T cells, B cells, CAR T cells, T reg cells, NK cells, NKT cells, TILs, skeletal muscle stem cells, cardiac stem cells, mesenchymal stem cells, cardiomyocytes, neurons, lymphocytes, macrophages, dendritic cells, and pancreatic islet cells.

In some embodiments, which may be combined with any of the preceding aspects or embodiments, the allogeneic cells are T cells.

In certain embodiments, the T cells comprise a chimeric antigen receptor (CAR) T cell, a universal CAR T cell, a split CAR T cell, an activatable CAR T cell, a repressible CAR T cell, a multiphasic CAR T cell, a tumor infiltrating lymphocyte, a regulatory T cell, a genetically modified T cell, a T cell with genetically modified or synthesized T cell receptors (TCRs), or virus-specific T cells.

In certain embodiments, the allogeneic cells are hematopoietic stem cells.

In some embodiments, which may be combined with any of the preceding aspects or embodiments, the allogeneic cells comprise allogeneic cells that are genetically distinguishable from each other.

In some embodiments, which may be combined with any of the preceding aspects or embodiments, the panel of SNPs comprises SNPs that have characteristics selected from the group consisting of: an overall population minor allele frequency of >0.4, a target population minor allele frequency of >0.4, the lowest polymerase error rate of the 6 potential allele transitions or transversions, and a genomic distance between each independent SNP of >500 kb.

In some embodiments, which may be combined with any of the preceding embodiments, the panel of SNPs comprises independent SNPs selected from the group consisting of: rs987640, rs1078004, rs6564027, rs2391110, rs2253592, rs2122080, rs1374570, rs57010808, rs7048541, rs1554472, rs1411271, rs475002, rs9471364, rs7825, rs12529, rs899076, rs8087320, rs10232552, rs1126899, rs909404, rs1052637, rs2175957, rs9951171, rs2245285, rs10743071, rs1051614, rs7017671, rs7284876, rs743616, rs1056149, rs3951216, rs1045644, rs28402995, rs5746846, rs1898882, rs6682717, rs4721083, rs6049836, rs7633246, rs6811238, rs10773760, rs9556269, rs11210490, rs1889819, rs13436, rs1055851, rs11560324, rs4775444, rs4302336, rs7182758, rs10192076, rs7306251, rs1411711, rs9914372, rs13428, rs2229627, rs13281208, rs2275047, rs561930, rs436278, rs3935070, rs1696455, rs1420398, rs13184586, rs1027895, rs10092491, rs344141, rs2255301, rs11126691, rs7173538, rs2070426, rs7161563, rs2099875, rs8058696, rs1600, rs57594411, rs6444724, rs1565933, rs12135784, rs2811231, rs6472465, rs4834806, rs993934, rs2833736, rs6094809, rs1151687, rs6918698, rs10826653, rs2180314, rs745142, rs2294092, rs12797748, rs12321981, rs12901575, rs9379164, rs11019968, rs4958153, rs1678690, rs8070085, rs6790129, rs4843371, rs2291395, rs9393728, rs868254, rs10918072, rs7451713, rs1352640, rs445251, rs3829655, rs9908701, rs1056033, rs4425547, rs1897820, rs1130857, rs4940019, rs34393853, rs2292830, rs11882583, rs9931073, rs12739002, rs11069797, rs7289, rs6807362, rs6492840, rs2509943, rs7526132, rs1522662, rs3129207, rs4806433, rs3802265, rs57985219, rs523104, rs2398849, rs7613749, rs7822225, rs10274334, rs1045248, rs35958120, rs10865922, rs2835296, rs12994875, rs2455230, rs625223, rs2281098, rs7112538, rs3748930, rs4571557, rs4733017, rs35596415, rs9640283, rs9865242, rs2295005, rs3810483, rs2248490, rs464663, rs2571028, rs1288207, rs61202512, rs2498982, rs12309796, rs4843380, rs2279665, rs36657, rs2269355, rs7009153, rs4666736, rs9843077, rs3816800, rs638405, rs3088241, rs590162, rs6443202, rs12646548, rs7315223, rs4501824, rs891700, rs1476864, rs7626681, rs76285932, rs79740603, rs3205187, rs6495680, rs740598, rs13182883, rs13218440, rs321198, rs1019029, rs9905977, rs13134862, rs1109037, rs1049544, rs1547202, rs55843637, rs1736442, rs1872575, rs12997453, rs4606077, rs9790986, rs1498553, rs2227910, rs62490396, rs2292972, rs733398, rs62485328, rs3790993, rs3793945, rs6591147, rs10776839, rs1679815, rs314598, rs12480506, rs6578843, rs9906231, rs10060772, rs901398, rs2007843, rs936019, rs648802, rs28756099, rs214955, rs10817691, rs1523537, rs9866013, rs12146092, rs234650, rs11776427, rs10503926, rs6719427, rs7853852, rs4288409, rs3731877, rs2289751, rs1779866, rs10932185, rs8097, rs7163338, rs12165004, rs3813609, rs985492, rs11106, rs528557, rs2270529, rs12237048, rs6459166, rs4510896, rs2503667, rs2567608, rs1047979, rs41317515, rs3173615, rs7785899, rs4849167, rs408600, rs1477239, rs3780962, rs12547045, rs9464704, rs2297236, rs2505232, rs6838248, rs7029934, rs2279776, rs3740199, rs3803798, rs1340562, rs4688094, rs7311115, rs2229571, rs159606, rs6955448, rs430046, rs17472365, rs3734311, rs7730991, rs2296545, rs12550831, rs6507284, rs254255, rs2733595, rs3812571, rs279844, rs2519123, rs7902629, rs9861037, rs1941230, rs3814182, rs2833622, rs560681, rs2071888, rs4936415, rs7589684, rs576261, rs9262, rs6907219, rs9289122, rs178649, rs208815, rs17818255, rs282338, rs2342767, rs3735615, rs10066756, rs75330257, rs6570914, rs3817687, rs2267234, rs7332388, rs315791, rs8004200, rs2075322, rs2121302, rs4803502, rs10831567, rs521861, rs10488710, rs903369, rs12680079, rs2272998, rs2302443, rs362124, rs10421285, rs6478448, rs7639794, rs2721150, rs259554, rs10500617, rs2358286, rs8025851, rs3848730, rs342910, rs1478829, rs726009, rs2182241, rs150079, rs1064074, rs6766396, rs7601771, rs1894252, rs1127472, rs6055803, rs977070, rs3751066, rs8076632, rs6508485, rs10496031, rs609521, rs1974855, rs35338631, rs1915632, rs8019787, rs2964164, rs7843841, rs6788347, rs6510057, rs2469523, rs12709176, rs9638798, rs7070730, rs12793830, rs2657167, rs7667167, rs2946994, rs2480345, rs3118957, rs10750524, rs7301328, rs722290, rs2289818, rs16964068, rs1821380, rs1112679, rs3190321, rs11648453, rs7205345, rs1049379, rs4890012, rs11081203, rs1048290, rs3826709, rs14155, rs4845480, rs874881, rs1044010, rs76275398, rs7543016, rs6101217, rs2056844, rs9617448, rs1317808, rs12713118, rs2717225, rs357483, rs14080, rs4680782, rs4364205, rs6794, rs10013388, rs1477898, rs11934579, rs448012, rs30353, rs73714299, rs7825714, rs10760016, and rs13295990.

In certain embodiments, the panel of SNPs comprises about 200 to about 210, about 210 to about 220, about 220 to about 230, about 230 to about 240, about 240 to about 250, about 250 to about 260, about 270 to about 280, about 280 to about 290, about 290 to about 300, about 300 to about 310, about 310 to about 320, about 320 to about 330, about 330 to about 340, about 340 to about 350, about 350 to about 360, about 360 to about 370, about 370 to about 380, about 380 to about 390, about 390 to about 400, or about 400 to 405 of the independent SNPs.

In certain embodiments, the panel of SNPs comprises rs987640, rs1078004, rs6564027, rs2391110, rs2253592, rs2122080, rs1374570, rs57010808, rs7048541, rs1554472, rs1411271, rs475002, rs9471364, rs7825, rs12529, rs899076, rs8087320, rs10232552, rs1126899, rs909404, rs1052637, rs2175957, rs9951171, rs2245285, rs10743071, rs1051614, rs7017671, rs7284876, rs743616, rs1056149, rs3951216, rs1045644, rs28402995, rs5746846, rs1898882, rs6682717, rs4721083, rs6049836, rs7633246, rs6811238, rs10773760, rs9556269, rs11210490, rs1889819, rs13436, rs1055851, rs11560324, rs4775444, rs4302336, rs7182758, rs10192076, rs7306251, rs1411711, rs9914372, rs13428, rs2229627, rs13281208, rs2275047, rs561930, rs436278, rs3935070, rs1696455, rs1420398, rs13184586, rs1027895, rs10092491, rs344141, rs2255301, rs11126691, rs7173538, rs2070426, rs7161563, rs2099875, rs8058696, rs1600, rs57594411, rs6444724, rs1565933, rs12135784, rs2811231, rs6472465, rs4834806, rs993934, rs2833736, rs6094809, rs1151687, rs6918698, rs10826653, rs2180314, rs745142, rs2294092, rs12797748, rs12321981, rs12901575, rs9379164, rs11019968, rs4958153, rs1678690, rs8070085, rs6790129, rs4843371, rs2291395, rs9393728, rs868254, rs10918072, rs7451713, rs1352640, rs445251, rs3829655, rs9908701, rs1056033, rs4425547, rs1897820, rs1130857, rs4940019, rs34393853, rs2292830, rs11882583, rs9931073, rs12739002, rs11069797, rs7289, rs6807362, rs6492840, rs2509943, rs7526132, rs1522662, rs3129207, rs4806433, rs3802265, rs57985219, rs523104, rs2398849, rs7613749, rs7822225, rs10274334, rs1045248, rs35958120, rs10865922, rs2835296, rs12994875, rs2455230, rs625223, rs2281098, rs7112538, rs3748930, rs4571557, rs4733017, rs35596415, rs9640283, rs9865242, rs2295005, rs3810483, rs2248490, rs464663, rs2571028, rs1288207, rs61202512, rs2498982, rs12309796, rs4843380, rs2279665, rs36657, rs2269355, rs7009153, rs4666736, rs9843077, rs3816800, rs638405, rs3088241, rs590162, rs6443202, rs12646548, rs7315223, rs4501824, rs891700, rs1476864, rs7626681, rs76285932, rs79740603, rs3205187, rs6495680, rs740598, rs13182883, rs13218440, rs321198, rs1019029, rs9905977, rs13134862, rs1109037, rs1049544, rs1547202, rs55843637, rs1736442, rs1872575, rs12997453, rs4606077, rs9790986, rs1498553, rs2227910, rs62490396, rs2292972, rs733398, rs62485328, rs3790993, rs3793945, rs6591147, rs10776839, rs1679815, rs314598, rs12480506, rs6578843, rs9906231, rs10060772, rs901398, rs2007843, rs936019, rs648802, rs28756099, rs214955, rs10817691, rs1523537, rs9866013, rs12146092, rs234650, rs11776427, rs10503926, rs6719427, rs7853852, rs4288409, rs3731877, rs2289751, rs1779866, rs10932185, rs8097, rs7163338, rs12165004, rs3813609, rs985492, rs11106, rs528557, rs2270529, rs12237048, rs6459166, rs4510896, rs2503667, rs2567608, rs1047979, rs41317515, rs3173615, rs7785899, rs4849167, rs408600, rs1477239, rs3780962, rs12547045, rs9464704, rs2297236, rs2505232, rs6838248, rs7029934, rs2279776, rs3740199, rs3803798, rs1340562, rs4688094, rs7311115, rs2229571, rs159606, rs6955448, rs430046, rs17472365, rs3734311, rs7730991, rs2296545, rs12550831, rs6507284, rs254255, rs2733595, rs3812571, rs279844, rs2519123, rs7902629, rs9861037, rs1941230, rs3814182, rs2833622, rs560681, rs2071888, rs4936415, rs7589684, rs576261, rs9262, rs6907219, rs9289122, rs178649, rs208815, rs17818255, rs282338, rs2342767, rs3735615, rs10066756, rs75330257, rs6570914, rs3817687, rs2267234, rs7332388, rs315791, rs8004200, rs2075322, rs2121302, rs4803502, rs10831567, rs521861, rs10488710, rs903369, rs12680079, rs2272998, rs2302443, rs362124, rs10421285, rs6478448, rs7639794, rs2721150, rs259554, rs10500617, rs2358286, rs8025851, rs3848730, rs342910, rs1478829, rs726009, rs2182241, rs150079, rs1064074, rs6766396, rs7601771, rs1894252, rs1127472, rs6055803, rs977070, rs3751066, rs8076632, rs6508485, rs10496031, rs609521, rs1974855, rs35338631, rs1915632, rs8019787, rs2964164, rs7843841, rs6788347, rs6510057, rs2469523, rs12709176, rs9638798, rs7070730, rs12793830, rs2657167, rs7667167, rs2946994, rs2480345, rs3118957, rs10750524, rs7301328, rs722290, rs2289818, rs16964068, rs1821380, rs1112679, rs3190321, rs11648453, rs7205345, rs1049379, rs4890012, rs11081203, rs1048290, rs3826709, rs14155, rs4845480, rs874881, rs1044010, rs76275398, rs7543016, rs6101217, rs2056844, rs9617448, rs1317808, rs12713118, rs2717225, rs357483, rs14080, rs4680782, rs4364205, rs6794, rs10013388, rs1477898, rs11934579, rs448012, rs30353, rs73714299, rs7825714, rs10760016, and rs13295990.

In some embodiments, which may be combined with any of the preceding aspects or embodiments, the level of allogeneic cell DNA is determined using the conditional probability of Bayesian probability theorem P(A|B)=P(B|A)*P(A)/P(B), assuming Mendelian genetics, and incorporating biallelic and high population minor allele frequency features of the panel of SNPs.

In certain embodiments, the method minimizes the use of prior distribution assumptions.

In another aspect, the present disclosure provides a method of assessing therapeutic effectiveness of allogeneic cells in a recipient of allogeneic cells, the method comprising: a) providing cell DNA from a sample obtained from the recipient of allogeneic cells; b) sequencing a panel of single nucleotide polymorphisms (SNPs) from the cell DNA, wherein the panel of SNPs is suitable for differentiating between allogeneic cell DNA and recipient-derived cell DNA; c) assaying differences in SNP allele distribution patterns in the panel as compared to expected homozygous or heterozygous distribution patterns to determine the level of allogeneic cell DNA; and d) assessing therapeutic effectiveness based on the level of allogeneic cell DNA. In some embodiments, a level of allogeneic cell DNA above a therapeutically effective threshold and/or increasing or stable over a time interval indicates therapeutic effectiveness, and a level of allogeneic cell DNA below a therapeutically effective threshold and/or decreasing over a time interval indicates a lack of therapeutic effectiveness.

In another aspect, the present disclosure provides a method of providing information on the status of allogeneic cells in a recipient, the method comprising: a) providing cell DNA from a sample obtained from the recipient; b) sequencing a panel of single nucleotide polymorphisms (SNPs) from the cell DNA, wherein the panel of SNPs is suitable for differentiating between allogeneic cell DNA and recipient-derived cell DNA; c) assaying differences in SNP allele distribution patterns in the panel as compared to expected homozygous or heterozygous distribution patterns to determine the level of allogeneic cell DNA; wherein a level of allogeneic cell DNA above a therapeutically effective threshold and/or increasing or stable over a time interval indicates a status of engraftment, expansion and/or persistence of the allogeneic cells, and a level of allogeneic cell DNA below a therapeutically effective threshold and/or decreasing over a time interval indicates a status of exhaustion, contraction, loss of persistence, allogeneic cell rejection, and/or graft vs. host disease; and d) providing information on the status of the allogeneic cells.

In another aspect, the present disclosure provides a method of providing information on a need to guide treatment of a recipient of allogeneic cells, the method comprising: a) providing cell DNA from a sample obtained from the recipient of allogeneic cells; b) sequencing a panel of single nucleotide polymorphisms (SNPs) from the cell DNA, wherein the panel of SNPs is suitable for differentiating between allogeneic cell DNA and recipient-derived cell DNA; c) assaying differences in SNP allele distribution patterns in the panel as compared to expected homozygous or heterozygous distribution patterns to determine the level of allogeneic cell DNA; wherein a level of allogeneic cell DNA above a therapeutically effective threshold and/or increasing or stable over a time interval indicates a status of engraftment, expansion and/or persistence of the allogeneic cells, and a level of allogeneic cell DNA below a therapeutically effective threshold and/or decreasing over a time interval indicates a status of exhaustion, contraction, loss of persistence, allogeneic cell rejection, and/or graft vs. host disease; and d) providing information on a need to guide treatment of the recipient.

In another aspect, the present disclosure provides a method of guiding treatment of a recipient of allogeneic cells, the method comprising: a) providing cell DNA from a sample obtained from the recipient of allogeneic cells; b) sequencing a panel of single nucleotide polymorphisms (SNPs) from the cell DNA, wherein the panel of SNPs is suitable for differentiating between allogeneic cell DNA and recipient-derived cell DNA; c) assaying differences in SNP allele distribution patterns in the panel as compared to expected homozygous or heterozygous distribution patterns to determine the level of allogeneic cell DNA; wherein a level of allogeneic cell DNA above a therapeutically effective threshold and/or increasing or stable over a time interval indicates a status of engraftment, expansion and/or persistence of the allogeneic cells, and a level of allogeneic cell DNA below a therapeutically effective threshold and/or decreasing over a time interval indicates a status of exhaustion, contraction, loss of persistence, allogeneic cell rejection, and/or graft vs. host disease; and d) guiding treatment of the recipient of allogeneic cells.

In another aspect, the present disclosure provides a method of determining the status of allogeneic cells in a recipient and having treatment of the recipient adjusted, the method comprising: a) providing cell DNA from a sample obtained from the recipient; b) sequencing a panel of single nucleotide polymorphisms (SNPs) from the cell DNA, wherein the panel of SNPs is suitable for differentiating between allogeneic cell DNA and recipient-derived cell DNA; c) assaying differences in SNP allele distribution patterns in the panel as compared to expected homozygous or heterozygous distribution patterns to determine the level of allogeneic cell DNA; wherein a level of allogeneic cell DNA above a therapeutically effective threshold and/or increasing or stable over a time interval indicates a status of engraftment, expansion and/or persistence of the allogeneic cells, and a level of allogeneic cell DNA below a therapeutically effective threshold and/or decreasing over a time interval indicates a status of exhaustion, contraction, loss of persistence, allogeneic cell rejection, and/or graft vs. host disease; and d) having treatment of the recipient adjusted.

In some embodiments, which may be combined with any of the preceding aspects or embodiments, the recipient of allogeneic cells received allogeneic cells from one source only.

In some embodiments, which may be combined with any of the preceding aspects or embodiments, wherein the recipient of allogeneic cells received allogeneic cells from two genetically related or unrelated sources.

### DESCRIPTION OF THE FIGURES

FIG. 1 illustrates that the percentage of cell DNA can be measured accurately in a mixture of DNA extracted from two different human cell lines. Genomic DNAs from two unrelated individuals (NA16689 and NA17070) were combined and serially diluted with NA17070 genomic DNA to generate “spike-in cell mixtures” that represented allogeneic cell to recipient cell ratios of 0, 0.01, 0.03, 0.1, 0.3, 0.6, 1, 3, 5, 10, 15, 20, 25, 20, 35, 40, 45, 50 and 100%. Generated mixtures were each analyzed in 5 replicates. The percentage of DNA from one genotype (“Expected”) is indicated on the x-axis. The percentage of spiked-in cell DNA was measured as expressed herein, and is shown on the y-axis (“% Spiked-in Cell DNA”). As indicated by the dotted lines, the plot at right shows a portion of the plot at left at a higher resolution.

FIG. 2 provides a schematic graph showing the typical level of allogeneic CAR T cells in a recipient over time. Time is indicated on the x-axis, with the time of CAR T cell administration to the recipient (“Infusion”), tₘₐₓ, 2 weeks following administration (“2 wks”), and months and/or years (“Months/years”) following administration noted. The percentage of allogeneic cells (“% Cell Product”) is indicated on the y-axis. As noted on the graph, following administration, the CAR T cells typically redistribute, expand, reach a Cₘₐₓ at a tₘₐₓ, contract, and then persist in the recipient.

FIG. 3 shows the results of measuring the proportion of DNA from the minor contributor in a blood panel made up of blood samples from a parent and child (panel AB). The x-axis shows the percentage of blood added from the minor contributor (“Expected % Minor Contributor”), and the y-axis shows the measured percentage of DNA from the minor contributor (“Measured % Minor Contributor”). As indicated on the plot, the R² value was 1.000.

FIG. 4 shows the results of measuring the proportion of DNA from the minor contributor in a blood panel made up of blood samples from unrelated individuals (panel CD). The x-axis shows the percentage of blood added from the minor contributor (“Expected % Minor Contributor”), and the y-axis shows the measured percentage of DNA from the minor contributor (“Measured % Minor Contributor”). As indicated on the plot, the R² value was 1.000.

FIG. 5 shows the results of measuring the proportion of DNA from the minor contributor in a blood panel made up of blood samples from unrelated individuals (panel EF). The x-axis shows the percentage of blood added from the minor contributor (“Expected % Minor Contributor”), and the y-axis shows the measured percentage of DNA from the minor contributor (“Measured % Minor Contributor”). As indicated on the plot, the R² value was 0.999.

FIG. 6 shows the results of measuring the proportion of DNA from the minor contributor in a blood panel made up of blood samples from unrelated individuals (panel GH). The x-axis shows the percentage of blood added from the minor contributor (“Expected % Minor Contributor”), and the y-axis shows the measured percentage of DNA from the minor contributor (“Measured % Minor Contributor”). As indicated on the plot, the R² value was 1.000.

FIG. 7 shows the results of measuring the proportion of DNA from the minor contributor in a blood panel made up of blood samples from unrelated individuals (panel IJ). The x-axis shows the percentage of blood added from the minor contributor (“Expected % Minor Contributor”), and the y-axis shows the measured percentage of DNA from the minor contributor (“Measured % Minor Contributor”). As indicated on the plot, the R² value was 1.000.

FIG. 8 shows the level of chimerism in CD3+ cells in 10 independent blood mixtures that were each generated by mixing blood from two unrelated individuals. CD3+ cells were enriched from whole blood using anti-CD3 antibodies and magnetic beads purification. DNA was extracted from CD3+ cells and the analysis was performed with high (100-150 ng DNA; closed squares) or low (8 ng DNA; open triangles) amount of DNA input. The x-axis indicates the sample name, and the y-axis indicates the percentage of chimerism measured.

FIG. 9 shows the level of chimerism in CD15+ cells in 10 independent blood mixtures that were each generated by mixing blood from two unrelated individuals. CD15+ cells were enriched from whole blood using anti-CD15 antibodies and magnetic beads purification. DNA was extracted from CD15+ cells and the analysis was performed with high (100-150 ng DNA; closed squares) or low (8 ng DNA; open triangles) amount of DNA input. The x-axis indicates the sample name, and the y-axis indicates the percentage of chimerism measured.

FIG. 10 shows the level of chimerism in CD33+ cells in 10 independent blood mixtures that were each generated by mixing blood from two unrelated individuals. CD33+ cells were enriched from whole blood using anti-CD33 antibodies and magnetic beads purification. DNA was extracted from CD33+ cells and the analysis was performed with high (100-150 ng DNA; closed squares) or low (8 ng DNA; open triangles) amount of DNA input. The x-axis indicates the sample name, and the y-axis indicates the percentage of chimerism measured.

FIG. 11 shows the level of chimerism in CD3+ cells in 10 independent blood mixtures that were each generated by mixing blood from two unrelated individuals. CD3+ cells were enriched from whole blood using anti-CD3 antibodies and magnetic beads purification. DNA was extracted from CD3+ cells and the analysis was performed with high (100-150 ng DNA; closed squares and asterisk) or low (8 ng DNA; open triangles and diamonds) amount of DNA input. DNA extracted from whole blood (squares and triangles) or buccal swabs (asterisks and diamonds) was used as a reference genome. The x-axis indicates the sample name, and the y-axis indicates the percentage of chimerism measured.

FIG. 12 shows the level of chimerism in CD15+ cells in 10 independent blood mixtures that were each generated by mixing blood from two unrelated individuals. CD15+ cells were enriched from whole blood using anti-CD15 antibodies and magnetic beads purification. DNA was extracted from CD15+ cells and the analysis was performed with high (100-150 ng DNA; closed squares and asterisk) or low (8 ng DNA; open triangles and diamonds) amount of DNA input. DNA extracted from whole blood (squares and triangles) or buccal swabs (asterisks and diamonds) was used as a reference genome. The x-axis indicates the sample name, and the y-axis indicates the percentage of chimerism measured.

FIG. 13 shows the level of chimerism in CD33+ cells in 10 independent blood mixtures that were each generated by mixing blood from two unrelated individuals. CD33+ cells were enriched from whole blood using anti-CD33 antibodies and magnetic beads purification. DNA was extracted from CD33+ cells and the analysis was performed with high (100-150 ng DNA; closed squares and asterisk) or low (8 ng DNA; open triangles and diamonds) amount of DNA input. DNA extracted from whole blood (squares and triangles) or buccal swabs (asterisks and diamonds) was used as a reference genome. The x-axis indicates the sample name, and the y-axis indicates the percentage of chimerism measured.

FIG. 14 shows the results of measuring the proportion of DNA in mixtures of DNA from three unrelated individuals (panel C). The three plots show the results for each of the three individuals (DNA1, DNA2, and DNA3). In each plot, the x-axis shows the expected percentage of DNA, and the y-axis shows the measured percentage of DNA.

FIG. 15 shows the results of measuring the proportion of DNA in mixtures of DNA from three unrelated individuals (panel D). The three plots show the results for each of the three individuals (DNA1, DNA2, and DNA3). In each plot, the x-axis shows the expected percentage of DNA, and the y-axis shows the measured percentage of DNA.

FIG. 16 shows the results of measuring the proportion of DNA in mixtures of DNA from two related individuals (parent and child) and one unrelated individual (panel G). The three plots show the results for each of the three individuals (DNA1, DNA2, and DNA3). In each plot, the x-axis shows the expected percentage of DNA, and the y-axis shows the measured percentage of DNA.

FIG. 17 shows the results of measuring the proportion of DNA in mixtures of DNA from two related individuals (parent and child) and one unrelated individual (panel H). The three plots show the results for each of the three individuals (DNA1, DNA2, and DNA3). In each plot, the x-axis shows the expected percentage of DNA, and the y-axis shows the measured percentage of DNA.

### DETAILED DESCRIPTION

The following description is presented to enable a person of ordinary skill in the art to make and use the various embodiments. Descriptions of specific devices, techniques, and applications are provided only as examples. Various modifications to the examples described herein will be readily apparent to those of ordinary skill in the art, and the general principles defined herein may be applied to other examples and applications without departing from the spirit and scope of the various embodiments. Thus, the various embodiments are not intended to be limited to the examples described herein and shown, but are to be accorded the scope consistent with the claims.

All references cited herein, including patent applications and publications, are hereby incorporated by reference in their entirety.

### Definitions

As used herein, the term “donor” refers to a subject genetically distinguishable from a “recipient” wherein the “donor” provides allogeneic cells for administration. As used herein, the phrase “allogeneic cells” refers to cells that originate from a donor. These include but are not limited to cells taken directly from a donor for administration into a recipient, cells taken from a donor and genetically engineered before administration into a recipient, cells taken from a donor and cultured before administration into a recipient, cells taken from a donor and subjected to a manufacturing process before administration into a recipient, and any combination thereof. Cells may also be stored before administration into a recipient (i.e., “off-the-shelf” cells).

As used herein, the term “engraftment” refers to the process by which allogeneic cells become established in a recipient. Engraftment occurs over the course of the first few days or weeks following administration of the allogeneic cells. Cells that fail to engraft may do so because they are rejected, as described below, or because they otherwise fail to survive in the recipient.

As used herein, the term “expansion” refers to the growth of allogeneic cells in a recipient after they have engrafted.

As used herein, the term “Cₘₐₓ” refers to the maximum level or percentage of allogeneic cells in a recipient following administration, engraftment, and expansion. Cₘₐₓ is an example of a cellular kinetic parameter. The time at which Cₘₐₓ is reached is herein termed “tₘₐₓ”.

As used herein, the term “contraction” refers to a decrease in the level or percentage of allogeneic cells in a recipient that occurs after the allogeneic cells reach Cₘₐₓ. Contraction occurs because cells are rejected, as described below, or otherwise fail to survive in the recipient.

As used herein, the term “persistence” refers to the ability of the allogeneic cell to survive and/or proliferate in the recipient. Persistence occurs over the course of months or years following administration of the allogeneic cells.

As used herein, the term “exhaustion” refers to the process by which allogeneic cells cease to perform their intended function in a recipient.

As used herein, the term “therapeutically effective threshold” refers to the minimum percentage or level of allogeneic cells that is indicative of allogeneic cell engraftment, expansion and/or persistence. The term “therapeutically effective threshold” may also refer to the minimum percentage or level of allogeneic cell DNA that is indicative of allogeneic cell engraftment, expansion and/or persistence. The therapeutically effective threshold of allogeneic cells or allogeneic cell DNA depends on the nature of the allogeneic cells and their indication.

As used herein, the terms “chimerism” and “level of chimerism” refer to the percentage or level of allogeneic cells in a recipient or sample compared to the total amount of allogeneic and recipient-derived cells in the recipient or sample. The terms “chimerism” and “level of chimerism” may also refer to the percentage or level of allogeneic cell DNA in a recipient or sample compared to the total amount of allogeneic and recipient-derived cell DNA in the recipient or sample.

As used herein, the term “area under the curve,” or “AUC” refers to the area under the curve of the level or percentage of allogeneic cells in a recipient over a time interval. The AUC is an example of a cellular kinetic parameter.

As used herein, the term “CAR T cell regulatory agent” refers to an agent (e.g., a drug or antibody) that inhibits or activates at least one activity of CAR T cells, or that kills or stimulates proliferation of CAR T cells.

### Overview

The present disclosure relates to methods of determining the status of allogeneic cells, as well as methods of administering allogeneic cells to a recipient and adjusting treatment of the recipient, treating allogeneic cell rejection in a recipient, monitoring for relapse of a hematologic cancer in a recipient, measuring the level of chimerism in a sample, measuring a cellular kinetic parameter of allogeneic cells in a recipient, identifying allogeneic cells in a recipient, predicting recipient responsiveness to allogeneic cell administration, and identifying recipients at a higher risk for a side effect associated with allogeneic cell administration.

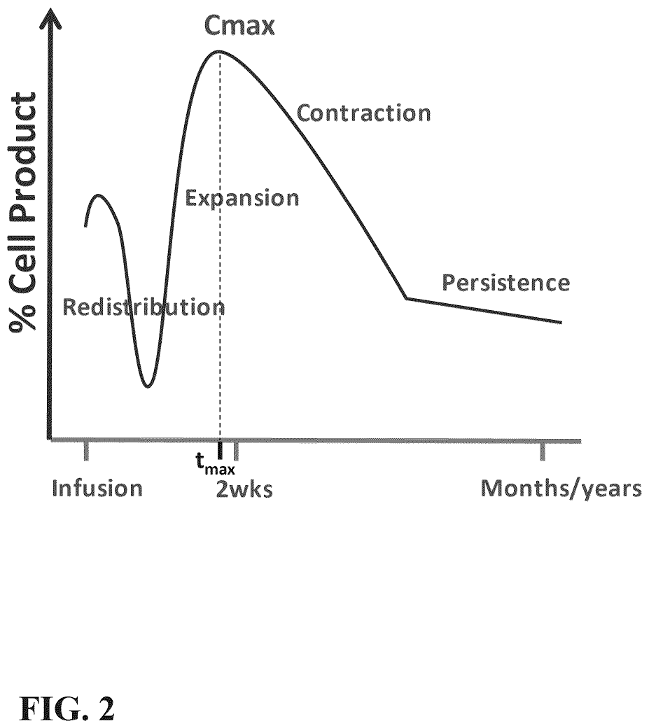

The present disclosure is based, at least in part, on Applicant's development of techniques for probing the status of allogeneic cells in a recipient. A recipient contains allogeneic cells that are foreign to the recipient's body. The allogeneic cells may engraft, expand, reach a Cₘₐₓ at a tₘₐₓ, contract, and persist in the recipient (FIG. 2). In another example, the allogeneic cells may engraft, expand and replace the recipient's immune system. The allogeneic cells may fail to survive in the recipient. In addition, the allogeneic cells may also trigger an immune response, which may lead to acute and/or chronic allogeneic cell rejection, including host vs. graft disease and graft vs. host disease. Without wishing to be bound by theory, it is thought that death and/or lack of replication of allogeneic cells results in a level of allogeneic cells that is lower than a therapeutically effective threshold of allogeneic cells and/or resulting in a decreasing level of allogeneic cells over a specific time interval.

Pre-conditioning treatments to deplete immune cells (e.g., lymphocytes) using chemotherapy or radiotherapy are sometimes required prior to allogeneic cell administration, in part, to reduce tumor burden and/or prevent or reduce the risk of an immune response resulting in allogeneic cell rejection. In cases where the allogeneic cells themselves are immune cells, allogeneic cells may replace all of the depleted recipient cells, resulting in complete cell chimerism (i.e., 100% chimerism). Alternatively, mixed chimerism (i.e., less than 100% chimerism) may occur in the following circumstances: when residual recipient immune cells survive pre-conditioning therapy, when no pre-conditioning therapy is performed, when a reduced intensity pre-conditioning therapy is administered to the recipient, if the allogeneic cells are experiencing rejection, or if disease relapse occurs. These circumstances may lead to the existence of both allogeneic and recipient cells in the recipient. Thus, cell DNA from a sample obtained from a recipient will contain cell DNA from allogeneic cells (i.e., administered allogeneic cell DNA) and/or cell DNA from the recipient's own cells (i.e., recipient cell DNA). Applicant's methods involve the analysis of cell DNA from the recipient to determine the level of allogeneic cell DNA in the recipient sample. This measurement may be used to assay the status of the allogeneic cells and/or determine the therapeutic effectiveness of allogeneic cells in the recipient. Typically, a level of allogeneic cell DNA below a therapeutically effective threshold and/or decreasing over a time interval indicates allogeneic cell exhaustion, contraction, loss of persistence, rejection, poor therapeutic effectiveness, and/or disease relapse. In contrast, a level of allogeneic cell DNA above a therapeutically effective threshold and/or increasing or stable over a specific time interval typically indicates allogeneic cell engraftment, expansion, persistence, and/or good therapeutic effectiveness. Determining the level of allogeneic cell DNA can thus be used to inform the recipient's course of treatment (e.g., adjusting the administration of allogeneic cells or otherwise adjusting the therapy being administered to the recipient).

To assay the status of allogeneic cells in the recipient, cell DNA can be extracted from a sample obtained from the recipient (e.g., whole blood or blood cells), and various polymorphic markers, such as single nucleotide polymorphism (SNP) loci, can be sequenced, where the panel of polymorphic markers, such as a panel of SNPs, is suitable for differentiating between allogeneic cell DNA and recipient cell DNA. The specific polymorphic markers selected to be on the panel include those that are identified as having low probabilities of being identical in any two individuals, thus making them appropriate for differentiating between recipient-derived cell DNA and allogeneic cell DNA. The number of polymorphic markers on the panel such as, for example, the number of SNPs on the panel, will be sufficient to discriminate between recipient and allogeneic alleles. The allele distribution patterns of polymorphic markers in the panel can be assayed to determine differences in distribution patterns as compared to expected homozygous (i.e., 0% or 100% of each allele) or heterozygous (i.e., 50% of each allele) distribution patterns, which can be used to determine the level of allogeneic cell DNA and, thus, the level of chimerism. Individual genotyping of the allogeneic cells and the recipient to determine which allele of the polymorphic locus belongs to the allogeneic cells and/or the recipient is not necessary, as differences in the polymorphic marker allele distribution pattern from expected homozygous or heterozygous distribution patterns inform the presence of allogeneic cell DNA in the population of cell DNA molecules isolated from the allogeneic cell recipient. In some cases, the majority signal from the cell DNA sample is allogeneic cell DNA and that the minority signal is recipient-derived DNA. In other cases, the majority signal from the cell DNA sample is recipient-derived DNA and that the minority signal is allogeneic cell DNA. Changes in the level of the allogeneic cell DNA over a specific time interval can be used to inform the status of the allogeneic cells in the recipient, as well as inform a need to adjust the treatment of the recipient. The methods of the present disclosure may be used to guide clinical decisions including decisions involving adjustments to the treatment of a recipient of allogeneic cells based on the status of the allogeneic cells.

Accordingly, the present disclosure provides methods of administering allogeneic cells to a recipient and adjusting treatment of the recipient, as well as determining the status of allogeneic cells, treating allogeneic cell rejection in a recipient, monitoring for relapse of a hematologic cancer in a recipient, measuring the level of chimerism in a sample, measuring a cellular kinetic parameter of allogeneic cells in a recipient, identifying allogeneic cells in a recipient, predicting recipient responsiveness to allogeneic cell administration, and identifying recipients at a higher risk for a side effect associated with allogeneic cell administration. The methods of the present disclosure may be used to guide decisions related to the adjustment of allogeneic cell administration and/or other treatments such as immunosuppressive therapy. In one aspect of the present disclosure, methods of administering allogeneic cells involve determining the level of allogeneic cell DNA in a sample from the recipient and adjusting treatment of the recipient based on the level of allogeneic cell DNA and/or the status of the allogeneic cells. In this case, cell DNA obtained from a recipient of allogeneic cells is provided, and a panel of SNPs suitable for differentiating between allogeneic cell DNA and recipient-derived cell DNA is sequenced. Differences in SNP allele distribution patterns compared to expected homozygous or heterozygous distribution patterns are assayed to determine the level of allogeneic cell DNA, and a level of allogeneic cell DNA above a therapeutically effective threshold and/or increasing or stable over a time interval indicates a status of engraftment, expansion and/or persistence of the allogeneic cells, and a level of allogeneic cell DNA below a therapeutically effective threshold and/or decreasing over a time interval indicates a status of exhaustion, contraction, loss of persistence, allogeneic cell rejection, and/or disease relapse. Treatment of the allogeneic cell recipient is then adjusted based on the status of the allogeneic cells.

The methods of the present disclosure may also be used to determine the status of allogeneic cells. This involves analyzing the level of allogeneic cell DNA in a recipient of allogeneic cells. In one aspect, methods of determining the status of allogeneic cells involve determining the level of allogeneic cell DNA in a sample obtained from a recipient of allogeneic cells using the methods described above. In this case, a level of allogeneic cell DNA above a therapeutically effective threshold and/or increasing or stable over a time interval indicates a status of engraftment, expansion and/or persistence of the allogeneic cells, and a level of allogeneic cell DNA below a therapeutically effective threshold and/or decreasing over a time interval indicates a status of exhaustion, contraction, loss of persistence, allogeneic cell rejection, and/or disease relapse. Based on this status, immunosuppressive therapy may be administered to the recipient or ongoing immunosuppressive therapy may be adjusted. Further, based on the status, the administration of the allogeneic cells may also be adjusted.

Some methods of the present disclosure may be used to treat allogeneic cell rejection, for example, by determining the level of allogeneic cell DNA in a sample obtained from a recipient of allogeneic cells using the methods described above in order to determine the status of the allogeneic cells in the recipient. Based on this status, administration of the allogeneic cells may be adjusted, and immunosuppressive therapy may be administered to the recipient or ongoing immunosuppressive therapy may be adjusted.

Another aspect of the present disclosure provides for methods that may be used to monitor for relapse of a hematologic cancer in a recipient. For example, the level of allogeneic cell DNA in a sample obtained from a recipient of allogeneic cells may be determined using the methods described above, and the therapeutic effectiveness of the allogeneic cells in the recipient may be determined based on the level of allogeneic cell DNA. Based on the level of allogeneic cell DNA, treatment may be re-initiated if there is a relapse of the hematologic cancer. Further, upon detection of a relapse, the method may further comprise withdrawal of an immune suppression therapy, administration of a chemotherapy, a second administration of allogeneic cells (e.g., an allogeneic cell transplantation), administration of a cytokine therapy, administration of an adoptive cell therapy, and/or donor lymphocyte infusion.

Some methods of the present disclosure involve measuring the level of chimerism in a sample. For example, the level of chimerism in a sample may be determined by providing cell DNA from a sample obtained from a recipient of allogeneic cells, sequencing a panel of single nucleotide polymorphisms (SNPs) from the cell DNA, wherein the panel of SNPs is suitable for differentiating between allogeneic cell DNA and recipient-derived cell DNA, assaying differences in SNP allele distribution patterns in the panel as compared to expected homozygous or heterozygous distribution patterns to determine the level of allogeneic cell DNA, and determining the level of chimerism in the sample. Based on the level of chimerism, treatment of the recipient may be adjusted.

Some methods of the present disclosure involve measuring a cellular kinetic parameter of allogeneic cells in a recipient. For example, a cellular kinetic parameter of allogeneic cells may be measured by determining the level of allogeneic cell DNA in a sample obtained from a recipient of allogeneic cells using the methods described above. Based on the cellular kinetic parameter, treatment of the recipient may be adjusted.

Some methods of the present disclosure involve identifying allogeneic cells in a recipient. For example, allogeneic cells in a recipient can be identified by providing cell DNA from a sample obtained from a recipient of allogeneic cells, sequencing a panel of single nucleotide polymorphisms (SNPs) from the cell DNA, wherein the panel of SNPs is suitable for differentiating between allogeneic cell DNA and recipient-derived cell DNA, and assaying differences in SNP allele distribution patterns in the panel as compared to expected homozygous or heterozygous distribution patterns to identify the allogeneic cell DNA.

Some methods of the present disclosure involve predicting recipient responsiveness to allogeneic cell administration. This can be determined, for example, by determining the level of allogeneic cell DNA in a sample obtained from a recipient of allogeneic cells using the methods described above. In some cases, a level of allogeneic cell DNA above a therapeutically effective threshold and/or increasing or stable over a time interval indicates that it is more likely that the recipient will respond to the allogeneic cells. In other cases, a level of allogeneic cell DNA below a therapeutically effective threshold and/or decreasing over a time interval indicates that it is less likely that the recipient will respond to the allogeneic cells.

Some methods of the present disclosure involve identifying recipients at a higher risk for a side effect associated with allogeneic cell administration. This can be determined, for example, by determining the level of allogeneic cell DNA in a sample obtained from a recipient of allogeneic cells using the methods described above. In some cases, a level of allogeneic cell DNA above a safety threshold indicates that the recipient is at a higher risk of a side effect associated with allogeneic cell administration. In these cases, the administration of allogeneic cells may be ceased, the recipient may be monitored more closely, and/or the recipient may be treated prophylactically. In other cases, a level of allogeneic cell DNA below a safety threshold indicates that the recipient is at a lower risk of a side effect associated with allogeneic cell administration.

Finally, some methods of the present disclosure relate to assessing therapeutic effectiveness of allogeneic cells in a recipient. Therapeutic effectiveness can be assessed, for example, by determining the level of allogeneic cell DNA in a sample obtained from a recipient of allogeneic cells using the methods described above. Assessing the therapeutic effectiveness of allogeneic cells in a recipient may be performed in the context of a clinical trial. Additional benefits and/or uses of the methods of the present disclosure will be readily apparent to one of skill in the art.

### Allogeneic Cells

The methods of the present disclosure involve providing cell DNA from a sample obtained from a subject who is the recipient of allogeneic cells. In this sense, the subject is a recipient of allogeneic cells who contains allogeneic cells and is typically a recipient of human allogeneic cells.

The recipient of allogeneic cells may have received one or more of a variety of allogeneic cells. Allogeneic cells may include, but are not limited to, blood cells, stem cells, cardiomyocytes, neurons, lymphocytes, NK cells, NKT cells, T reg cells, macrophages, dendritic cells, and pancreatic islet cells. In some embodiments, the allogeneic cells are allogeneic blood cells. Allogeneic blood cells may include hematopoietic stem cells (i.e., HSCs), T cells, B cells, and CAR T cells, NK cells, NKT cells, TILs. In some embodiments, the allogeneic cells are allogeneic T cells. In some embodiments, allogeneic cells are administered as bone marrow, cord blood, or purified allogeneic cells. In some embodiments, the allogeneic cells are bone marrow cells. In some embodiments, the allogeneic cells are cord blood cells. In some embodiments, the allogeneic cells are allogeneic CAR T cells, allogeneic universal CAR T cells (i.e., where the CAR binds to an antibody that binds a specific antigen), allogeneic split CAR T cells (i.e., where a dimerizing agent activates CAR T cell function), allogeneic activatable CAR T cells, allogeneic repressible CAR T cells, allogeneic multiphasic CAR T cells (i.e., where the CAR must bind multiple specific antigens and/or agents to induce T cell activation), allogeneic tumor infiltrating lymphocytes, allogeneic regulatory T cells, allogeneic genetically modified T cells, or allogeneic T cells with genetically modified or synthesized T cell receptors (TCRs), virus-specific T cells (e.g. EBV, HPV, BKV, CMV, etc.), antigen-specific T cells, neoantigen-specific T cells, or any cell isolated from a donor. In some embodiments, the allogeneic cells are derived from a donor. In some embodiments, the allogeneic cells are a cell allograft.

In some embodiments where the disclosed methods are used to determine the level of allogeneic cell DNA in a sample obtained from a recipient of allogeneic cells, the allogeneic cells are HSCs. In some embodiments, the HSCs are administered as bone marrow, cord blood, or purified HSCs. In some embodiments, the HSCs are derived from a donor. In some embodiments, the HSCs are administered as a hematopoietic cell transplantation.

In some embodiments where the disclosed methods are used to determine the level of allogeneic cell DNA in a sample obtained from a recipient of allogeneic cells, the allogeneic cells are allogeneic CAR T cells. Allogeneic CAR T cells are T cells that have been genetically modified to express polypeptides called CARs on their surface using any method known in the art for making CAR T cells. For exemplary methods, see Zhang et al. (2017) Biomarker Research 5(22):1-6; Fesnak, Davis, and Levine (2017) Nature Protocols 12(4).

CARs are engineered receptors that contain at least an extracellular antigen recognition domain (frequently a single-chain variable antibody fragment or human Fab fragment), a transmembrane domain, and an intracellular signaling domain derived from a T-cell receptor (TCR). Without wishing to be bound by theory, it is thought that when the antigen recognition domain binds its target antigen(s) (e.g., a cancer antigen), the intracellular domain of the CAR signals for T cell activation.

In some embodiments, the allogeneic cells are regulatable CAR T cells. These are CAR T cells that may be stimulated or inhibited upon addition of a CAR T cell regulatory agent. In some embodiments, the regulatable CAR T cells are made up of split CAR T cells, activatable CAR T cells, repressible CAR T cells, or multiphasic CAR T cells. In some embodiments where the allogeneic cells are regulatable CAR T cells, adjusting treatment of the allogeneic cells involves initiating administration of a CAR T cell regulatory agent or adjusting the dosage of a CAR T cell regulatory agent administered to the recipient.

In some embodiments of the methods of the present disclosure, the allogeneic cells are stem cells. In some embodiments, the allogeneic stem cells are embryonic, tissue-specific, mesenchymal, induced pluripotent, hematopoietic, mesenchymal, skeletal, myogenic, cardiac, neural, epidermal, or intestinal stem cells. In some embodiments, the allogeneic stem cells are hematopoietic stem cells.

In some embodiments, the allogeneic cells comprise multiple types of allogeneic cells. In some embodiments, the allogeneic cells comprise allogeneic cells that are genetically distinguishable from each other. In some embodiments, the allogeneic cells comprise two populations of genetically distinguishable allogeneic cells. The methods of the present disclosure may be used to determine the levels of two populations of allogeneic cells with different genotypes in a recipient. In some embodiments, there are two independent administrations of allogeneic cells. In some embodiments, allogeneic cells are administered in a first administration of allogeneic cells and a second administration of allogeneic cells. For example, in some embodiments, a first administration of allogeneic cells comprises HSCs, and a second administration of allogeneic cells comprises CAR T cells.

Samples from Recipient

In some embodiments, the provided sample from the recipient may include whole blood, whole blood cells, isolated blood cells, or DNA or other nucleic acids extracted from the whole blood, whole blood cells, or isolated blood cells. Isolated blood cells may include peripheral blood mononuclear cells (i.e., PMBCs), hematopoietic stem cells (i.e., HSCs), lymphoid progenitor cells, myeloid progenitor cells, white blood cells, granulocytes, agranulocytes, myeloblasts, basophils, eosinophils, neutrophils, mast cells, lymphocytes, monocytes, macrophages, nucleated red blood cells, erythroblasts, megakaryocytes, natural killer cells, B cells, T cells, helper T cells, inducer T cells, regulatory T cells, and cytotoxic T cells, NK cells, NKT cells. In some embodiments, the provided sample is whole blood or PMBCs from the recipient. In some embodiments, the provided sample is bone marrow.

Samples obtained from the recipient of allogeneic cells contain cell DNA. The total cell DNA present in the sample may be entirely allogeneic cell DNA, a mixture of recipient-derived cell DNA and allogeneic cell DNA, or entirely recipient-derived cell DNA.

Once a sample is obtained, it can be used directly, frozen, or otherwise stored in a condition that maintains the integrity of the cell DNA and prevents degradation and/or contamination of the sample. The amount of a sample that is taken at a particular time may vary and may depend on additional factors, such as any need to repeat analysis of the sample. In some embodiments, up to 50, 40, 30, 20, 10, 9, 8, 7, 6, 5, 4, 3, 2, 1, 0.5, 0.1 mL of a sample is obtained. In some embodiments, 0.1-1, 1-50, 2-40, 3-30, or 4-20 mL of a sample is obtained. In some embodiments, more than 1, 5, 10, 15, 20, 25, 30, 35, 40, 45, 50, 55, 60, 65, 70, 75, 80, 85, 90, 95, or 100 mL of a sample is obtained.

Samples may be obtained from a recipient of allogeneic cells once or more than once. Where multiple samples are to be obtained from a recipient of allogeneic cells, the frequency of sampling may vary. For example, samples may be obtained about once every day, once every other day, once every three days, about once every week, about once every two weeks, about once every three weeks, about once every month, about once every two months, about once every three months, about once every four months, about once every five months, about once every six months, about once every year, or about once every two years or more after the initial sampling event. One, two, three, four, five, ten, twenty, thirty, forty, fifty, sixty, seventy, eighty, ninety, one hundred, two hundred, three hundred, four hundred, or five hundred or more samples may be obtained from the recipient.

One or more samples may be obtained from a recipient of allogeneic cells over a time interval for use in determining the status of the allogeneic cells according to the methods of the present disclosure. The time interval during which samples are taken from the recipient of allogeneic cells following the allogeneic cell administration event may vary. For example, multiple samples may be obtained within one day, two days, three days, one week, two weeks, three weeks, one month, two months, three months, four months, five months, six months, one year, or two years or more after administering allogeneic cells to a recipient. In some embodiments, the time interval for obtaining samples from a recipient of allogeneic cells is within the first few days after administering allogeneic cells to a recipient. In some embodiments, samples are obtained within one to thirty days after administering allogeneic cells to a recipient. In some embodiments, samples are obtained within 1, 2, 3, 4, 5, 6, 7, 8, 9, 10, 11, 12, 13, 14, 15, 16, 17, 18, 19, 20, 21, 22, 23, 24, 25, 26, 27, 28, 29, or 30 days after administering allogeneic cells to a recipient. In some embodiments, the time interval for obtaining samples from an allogeneic cell is during tapering of an immunosuppressive regimen, an interval that occurs during the first 12 months after allogeneic cell administration to the recipient occurred. In some embodiments, the time interval for obtaining samples from a recipient of allogeneic cells is during the initial long term immunosuppressive maintenance phase, beginning about 12-14 months after the allogeneic cell administration to the recipient occurred. In some embodiments, the time interval for obtaining samples from a recipient of allogeneic cells is during the entire long term maintenance of the immunosuppressive regimen, any time beyond 12 months after the allogeneic cell administration to the recipient occurred.

In some embodiments, samples are obtained from a recipient of allogeneic cells twice a week in the first three weeks after administering allogeneic cells to a recipient. In some embodiments, samples are obtained daily for the first 1 or 2 weeks following administration. In some embodiments, samples are obtained once a week for the first three months after administering allogeneic cells to a recipient. In some embodiments, samples are obtained once a month for the first year after administering allogeneic cells to a recipient. In some embodiments, samples are obtained four times a year after the first year after administering allogeneic cells to a recipient.

In some embodiments, samples are obtained from a recipient of allogeneic cells for one to three consecutive months, starting at the one year anniversary of the allogeneic cell administration event (i.e. 12 months after the allogeneic cell administration event), providing a total of four to six samples for analysis taken over a three month time interval, with samples being collected about every two weeks. In some embodiments, a recipient of allogeneic cells has samples taken once a week for one to three consecutive months, starting at the one year anniversary of the allogeneic cell administration event (i.e. 12 months after the allogeneic cell administration event), providing a total of twelve samples for analysis taken over a three month time interval. The total duration of obtaining samples from a recipient of allogeneic cells, as well as the frequency of obtaining such samples, may vary and will depend on a variety of factors, such as clinical progress. For example, a recipient of allogeneic cells may have samples obtained for analysis of cell DNA for the duration of their lifetime. Appropriate timing and frequency of sampling will be able to be determined by one of skill in the art for a given recipient of allogeneic cells.

### Cell DNA

The methods of the present disclosure involve the analysis of cell DNA from a recipient of allogeneic cells to inform a need to adjust treatment being administered to the recipient of allogeneic cells. Cell DNA generally refers to DNA that is present inside of a cell such as, for example, DNA that is present in cells of a bodily fluid (e.g., whole blood) or DNA that is present in isolated cells (e.g., isolated blood cells) of a subject. Cell DNA may have originated from various locations within a cell, for example, from the cell nucleus or from mitochondria. Without wishing to be bound by theory, it is believed that allogeneic cell exhaustion, allogeneic cell contraction, loss of allogeneic cell persistence, allogeneic cell rejection, allogeneic cell apoptosis, necrosis of allogeneic cells, or disease relapse result in a level of allogeneic cells that is below a therapeutically effective threshold and/or is decreasing compared to a previously analyzed sample. Samples from recipients in which the level of allogeneic cells is below a therapeutically effective threshold and/or is decreasing may contain both the recipient's own endogenous cell DNA (recipient-derived cell DNA) and cell DNA that originated from the allogeneic cells, where the allogeneic cell DNA is below a therapeutically effective threshold and/or is decreasing over a specific time interval compared to a previously analyzed DNA sample. Correspondingly, without wishing to be bound by theory, it is believed that allogeneic cell engraftment, allogeneic cell expansion and/or persistence of the allogeneic cells results in a level of allogeneic cell DNA that is above a therapeutically effective threshold and/or increasing or stable compared to a previously analyzed sample. Detecting a change in the levels of allogeneic cell DNA in a recipient of allogeneic cells according to the methods of the present disclosure may be used to determine the status of the allogeneic cells and inform a need to adjust the recipient's therapy.

Various methods of isolating cell DNA are well-known in the art and described herein (e.g., methodologies such as those described in Sambrook et al. Molecular Cloning: A Laboratory Manual 3^{rd} edition (2001) Cold Spring Harbor Laboratory Press, Cold Spring Harbor, N.Y.; F. M. Ausubel, et al. eds., Current Protocols in Molecular Biology, latest edition). In some embodiments, cells contained in the sample are lysed, cell DNA is precipitated or bound by a column, then cell DNA is purified, for example, by washing the precipitated DNA or the DNA bound to the column and then resuspending or eluting the DNA with an appropriate buffer. In some embodiments, cell DNA may be extracted using a kit, for example, the QIAamp DNA Mini Kit (Qiagen Cat. No. 51306), Blood & Cell Culture DNA Mini Kit (Qiagen), DNA Isolation Kit for Cells and Tissues (Sigma-Aldrich), Monarch Genomic DNA Purification Kit (New England Biolabs), DNeasy Blood & Tissue Kit (Qiagen), Mitochondrial DNA Isolation Kit (Abcam), Nuclear Extraction Kit (Abcam), or another appropriate commercially available kit. In some embodiments, nuclear DNA is isolated from the sample. In some embodiments, mitochondrial DNA is isolated from the sample. In some embodiments, total DNA is isolated from the sample. Once the cell DNA is isolated, it can be used directly, frozen, or otherwise stored in a condition that maintains the integrity and prevents degradation and/or contamination of the cell DNA.

Cell RNA may also be isolated from a recipient of allogeneic cells and analyzed by analogous methods as described above and/or through analysis of recipient RNA levels from specific marker genes. Thus, the methods of the present disclosure generally relate to analysis of cellular nucleic acids from a recipient of allogeneic cells to determine the status of the allogeneic cells and/or to inform a need to adjust therapy being administered to the recipient of allogeneic cells.

### Analysis of Cell DNA

The methods of the present disclosure involve the analysis of cell DNA from a recipient of allogeneic cells. After cell DNA has been isolated from a recipient of allogeneic cells, various methods and techniques may be used to analyze the cell DNA, including sequencing a panel of SNPs from the cell DNA, wherein the panel of SNPs is suitable for differentiating between allogeneic cell DNA and recipient-derived cell DNA and assaying differences in SNP allele distribution patterns in the panel as compared to expected homozygous or heterozygous distribution patterns to determine the level of allogeneic cell DNA.

Analysis of cell DNA according to the methods of the present disclosure involves analysis of a panel of polymorphic markers from the cell DNA. In some embodiments, the polymorphic markers are SNPs. In some embodiments, SNPs are selected to be included in the panel at least in part on the basis that the panel of SNPs will be sufficient to differentiate between allogeneic cell DNA and recipient-derived cell DNA.

### Panels of Polymorphic Markers

Analysis of cell DNA obtained from a recipient of allogeneic cells involves the analysis of a panel of polymorphic markers from the cell DNA. Various polymorphic markers may be selected for inclusion in the panel to be analyzed as long as the polymorphic marker panel as a whole is suitable for differentiating between allogeneic cell DNA and recipient-derived cell DNA. The same polymorphic marker panel may be used for each recipient of allogeneic cells; there is no need to customize polymorphic marker panels to individualize the panel to different recipients of allogeneic cells.

Various types of polymorphic markers may be included in polymorphic marker panels. Polymorphic markers are found at a region of DNA containing a polymorphism. A polymorphism generally refers to the occurrence of two or more genetically determined alternative sequences or alleles in a population. A polymorphism may also refer to epigenetic differences between one or more nucleotides at a particular locus of the DNA, for example, the presence or absence of methylation on a particular nucleotide. A polymorphism may contain, for example, one or more base changes or modifications, an insertion, a repeat, or a deletion. A polymorphic locus may be as small as one base pair, such as a SNP. Polymorphic markers may include, for example, single nucleotide polymorphisms (SNPs), restriction fragment length polymorphisms (RFLPs), short tandem repeats (STRs), variable number of tandem repeats (VNTRs), hypervariable regions, minisatellites, dinucleotide repeats, trinucleotide repeats, tetranucleotide repeats, simple sequence repeats, and insertion elements. A polymorphism between two nucleic acids can be naturally occurring or may be caused by exposure to or contact with chemicals, enzymes, or other agents or exposure to agents that cause damage to nucleic acids, for example, ultraviolet radiation, mutagens, or carcinogens. Additional types of polymorphisms and polymorphic markers will be readily apparent to one of skill in the art.

Various combinations of polymorphic marker types may be used in polymorphic marker panels. For example, the polymorphic marker panel may include both SNPs and short tandem repeats or any other type of polymorphic marker. In some embodiments, the polymorphic marker panel is composed entirely of SNPs; thus, the polymorphic marker panel is a SNP panel. Additional combinations of polymorphic markers on polymorphic marker panels will be readily apparent to one of skill in the art.

Selection of the appropriate quantity and identity of polymorphic markers to be analyzed from cell DNA may vary, as will be appreciated by one of skill in the art. The panel of polymorphic markers to be analyzed may include at least 10, at least 15, at least 20, at least 25, at least 30, at least 35, at least 40, at least 45, at least 50, at least 55, at least 60, at least 65, at least 70, at least 75, at least 80, at least 85, at least 90, at least 95, at least 100, at least 105, at least 110, at least 115, at least 120, at least 150, at least 200, at least 250, at least 300, at least 350, at least 400, at least 450, at least 500, at least 1000, or at least 1500 or more independent polymorphic markers.

In some embodiments, the polymorphic marker panel is a panel of SNPs. SNPs to be included in the SNP panel, or in any other polymorphic marker panel, may be those previously identified as being suitable for differentiating between any two unrelated individuals (e.g., Pakstis et al., (2010) Hum Genet. 127(3):315-24). For example, the SNP panel may include one or more of the following human SNPs (named according to dbSNP numbering): rs987640, rs1078004, rs6564027, rs2391110, rs2253592, rs2122080, rs1374570, rs57010808, rs7048541, rs1554472, rs1411271, rs475002, rs9471364, rs7825, rs12529, rs899076, rs8087320, rs10232552, rs1126899, rs909404, rs1052637, rs2175957, rs9951171, rs2245285, rs10743071, rs1051614, rs7017671, rs7284876, rs743616, rs1056149, rs3951216, rs1045644, rs28402995, rs5746846, rs1898882, rs6682717, rs4721083, rs6049836, rs7633246, rs6811238, rs10773760, rs9556269, rs11210490, rs1889819, rs13436, rs1055851, rs11560324, rs4775444, rs4302336, rs7182758, rs10192076, rs7306251, rs1411711, rs9914372, rs13428, rs2229627, rs13281208, rs2275047, rs561930, rs436278, rs3935070, rs1696455, rs1420398, rs13184586, rs1027895, rs10092491, rs344141, rs2255301, rs11126691, rs7173538, rs2070426, rs7161563, rs2099875, rs8058696, rs1600, rs57594411, rs6444724, rs1565933, rs12135784, rs2811231, rs6472465, rs4834806, rs993934, rs2833736, rs6094809, rs1151687, rs6918698, rs10826653, rs2180314, rs745142, rs2294092, rs12797748, rs12321981, rs12901575, rs9379164, rs11019968, rs4958153, rs1678690, rs8070085, rs6790129, rs4843371, rs2291395, rs9393728, rs868254, rs10918072, rs7451713, rs1352640, rs445251, rs3829655, rs9908701, rs1056033, rs4425547, rs1897820, rs1130857, rs4940019, rs34393853, rs2292830, rs11882583, rs9931073, rs12739002, rs11069797, rs7289, rs6807362, rs6492840, rs2509943, rs7526132, rs1522662, rs3129207, rs4806433, rs3802265, rs57985219, rs523104, rs2398849, rs7613749, rs7822225, rs10274334, rs1045248, rs35958120, rs10865922, rs2835296, rs12994875, rs2455230, rs625223, rs2281098, rs7112538, rs3748930, rs4571557, rs4733017, rs35596415, rs9640283, rs9865242, rs2295005, rs3810483, rs2248490, rs464663, rs2571028, rs1288207, rs61202512, rs2498982, rs12309796, rs4843380, rs2279665, rs36657, rs2269355, rs7009153, rs4666736, rs9843077, rs3816800, rs638405, rs3088241, rs590162, rs6443202, rs12646548, rs7315223, rs4501824, rs891700, rs1476864, rs7626681, rs76285932, rs79740603, rs3205187, rs6495680, rs740598, rs13182883, rs13218440, rs321198, rs1019029, rs9905977, rs13134862, rs1109037, rs1049544, rs1547202, rs55843637, rs1736442, rs1872575, rs12997453, rs4606077, rs9790986, rs1498553, rs2227910, rs62490396, rs2292972, rs733398, rs62485328, rs3790993, rs3793945, rs6591147, rs10776839, rs1679815, rs314598, rs12480506, rs6578843, rs9906231, rs10060772, rs901398, rs2007843, rs936019, rs648802, rs28756099, rs214955, rs10817691, rs1523537, rs9866013, rs12146092, rs234650, rs11776427, rs10503926, rs6719427, rs7853852, rs4288409, rs3731877, rs2289751, rs1779866, rs10932185, rs8097, rs7163338, rs12165004, rs3813609, rs985492, rs11106, rs528557, rs2270529, rs12237048, rs6459166, rs4510896, rs2503667, rs2567608, rs1047979, rs41317515, rs3173615, rs7785899, rs4849167, rs408600, rs1477239, rs3780962, rs12547045, rs9464704, rs2297236, rs2505232, rs6838248, rs7029934, rs2279776, rs3740199, rs3803798, rs1340562, rs4688094, rs7311115, rs2229571, rs159606, rs6955448, rs430046, rs17472365, rs3734311, rs7730991, rs2296545, rs12550831, rs6507284, rs254255, rs2733595, rs3812571, rs279844, rs2519123, rs7902629, rs9861037, rs1941230, rs3814182, rs2833622, rs560681, rs2071888, rs4936415, rs7589684, rs576261, rs9262, rs6907219, rs9289122, rs178649, rs208815, rs17818255, rs282338, rs2342767, rs3735615, rs10066756, rs75330257, rs6570914, rs3817687, rs2267234, rs7332388, rs315791, rs8004200, rs2075322, rs2121302, rs4803502, rs10831567, rs521861, rs10488710, rs903369, rs12680079, rs2272998, rs2302443, rs362124, rs10421285, rs6478448, rs7639794, rs2721150, rs259554, rs10500617, rs2358286, rs8025851, rs3848730, rs342910, rs1478829, rs726009, rs2182241, rs150079, rs1064074, rs6766396, rs7601771, rs1894252, rs1127472, rs6055803, rs977070, rs3751066, rs8076632, rs6508485, rs10496031, rs609521, rs1974855, rs35338631, rs1915632, rs8019787, rs2964164, rs7843841, rs6788347, rs6510057, rs2469523, rs12709176, rs9638798, rs7070730, rs12793830, rs2657167, rs7667167, rs2946994, rs2480345, rs3118957, rs10750524, rs7301328, rs722290, rs2289818, rs16964068, rs1821380, rs1112679, rs3190321, rs11648453, rs7205345, rs1049379, rs4890012, rs11081203, rs1048290, rs3826709, rs14155, rs4845480, rs874881, rs1044010, rs76275398, rs7543016, rs6101217, rs2056844, rs9617448, rs1317808, rs12713118, rs2717225, rs357483, rs14080, rs4680782, rs4364205, rs6794, rs10013388, rs1477898, rs11934579, rs448012, rs30353, rs73714299, rs7825714, rs10760016, and rs13295990. In some embodiments, the panel of polymorphic markers to be analyzed may include at least 10, at least 15, at least 20, at least 25, at least 30, at least 35, at least 40, at least 45, at least 50, at least 55, at least 60, at least 65, at least 70, at least 75, at least 80, at least 85, at least 90, at least 95, at least 100, at least 105, at least 110, at least 115, at least 120, at least 150, at least 200, at least 250, at least 300, at least 350, or at least 400 of the 405 above mentioned SNPs. In some embodiments, each of the 405 above mentioned SNPs is included in the polymorphic marker panel.

In some embodiments, the SNP panel may include, for example, about 10 to about 20, about 20 to about 30, about 30 to about 40, about 40 to about 50, about 50 to about 60, about 60 to about 70, about 70 to about 80, about 80 to about 90, about 90 to about 100, about 100 to about 110, about 110 to about 120, about 120 to about 130, about 130 to about 140, about 140 to about 150, about 150 to about 160, about 160 to about 170, about 170 to about 180, about 180 to about 190, about 190 to about 200, about 200 to about 210, about 210 to about 220, about 220 to about 230, about 230 to about 240, about 240 to about 250, about 250 to about 260, about 270 to about 280, about 280 to about 290, about 290 to about 300, about 300 to about 310, about 310 to about 320, about 320 to about 330, about 330 to about 340, about 340 to about 350, about 350 to about 360, about 360 to about 370, about 370 to about 380, about 380 to about 390, about 390 to about 400, or about 400 to 405 of the 405 independent SNPs identified above.

In some embodiments, the SNP panel comprises more than 405 SNPs. In some embodiments, the SNP panel comprises at least 500, at least 600, at least 700, at least 800, at least 900, at least 1000, at least 1100, at least 1200, at least 1300, at least 1400, or at least 1500 SNPs.

SNPs may also be selected on the basis that they have, for example, an overall population minor allele frequency of >0.4, a target population minor allele frequency of >0.4, the lowest polymerase error rate (in the test system) of the 6 potential allele transitions or transversions, and/or low linkage on the genome such as, for example, >500 kb distance between each independent SNP. In some embodiments, SNPs are selected on the basis that they have an overall population minor allele frequency of >0.4. In some embodiments, SNPs are selected on the basis that they have a target population minor allele frequency of >0.4. In some embodiments, SNPs are selected on the basis that they have the lowest polymerase error rate (in the test system) of the 6 potential allele transitions or transversions. In some embodiments, SNPs are selected on the basis that they have low linkage on the genome such as, for example, >500 kb distance between each independent SNP. In some embodiments, SNPs are selected on the basis that they have one, two, three, or four of the above mentioned traits.

### Amplification and Sequencing

Cell DNA isolated from a recipient of allogeneic cells may be amplified for downstream techniques and analysis, such as analysis of a panel of polymorphic markers from the cell DNA. Methods of amplifying DNA are well-known in the art and are described herein. Amplification generally refers to any device, method, or technique that can generate copies of a nucleic acid. Amplification of cell DNA may involve, for example, polymerase chain reaction (PCR)-based methods such as standard PCR, hot-start PCR, multiplex PCR, GC-rich PCR, touchdown PCR, quantitative PCR, digital droplet PCR, and the like. The Fluidigm Access Array™ System, the RainDance Technologies RainDrop system, or other technologies for multiplex amplification may be used for multiplex or highly parallel simplex DNA amplification. Amplification may involve the use of high-fidelity polymerases such as, for example, FastStart High Fidelity (Roche), Expand High Fidelity (Roche), Phusion Flash II (ThermoFisher Scientific), Phusion Hot Start II (ThermoFisher Scientific), KAPA HiFi (Kapa BioSystems), or KAPA2G (Kapa Biosystems).

Amplification may include an initial PCR cycle that adds a unique sequence to each individual molecule, called molecular indexing. Unique sequences for molecular indexing can also be ligated to the DNA after amplification. Molecular indexing allows for quantitative assessment of the absolute level of both alleles for each SNP amplicon and therefore may improve precision and accuracy in determining the percentage of allogeneic cell DNA.

Amplified DNA may also be subjected to additional processes, such as sample indexing (also referred to as sample barcoding or tagging). Methods of sample indexing of DNA are well-known in the art and are described herein. Sample indexing allows for the use of multiplex-sequencing platforms, which are compatible with a variety of sequencing systems, such as Illumina HiSeq, MiSeq; ThermoFisher Scientific Ion PGM and Ion Proton; GenapSys Sequencer; and Oxford Nanopore Flongle, MinION, GridION, and PromethION. Multiplex sequencing permits the sequencing of DNA from multiple samples at once through the use of DNA indexing to specifically identify the sample source of the sequenced DNA.

The amount of DNA that is used for analysis may vary. In some embodiments, less than 1 pg, 5 pg, 10 pg, 20 pg, 30 pg, 40 pg, 50 pg, 100 pg, 200 pg, 500 pg, 1 ng, 5 ng, 10 ng, 20 ng, 30 ng, 40 ng, 50 ng, 100 ng, 200 ng, 500 ng, 1 μg, 5 μg, 10 μg, 20 μg, 30 μg, 40 μg, 50 μg, 100 μg, 200 μg, 500 μg, or 1 mg of DNA are obtained from the sample for further genetic analysis. In some cases, about 1-5 pg, 5-10 pg, 10-100 pg, 100 pg-1 ng, 1-5 ng, 5-10 ng, 10-100 ng, or 100 ng-1 μg of DNA are obtained from the sample for further genetic analysis.

The methods of the present disclosure involve sequencing target loci from cell DNA, as well as analyzing sequence data. Various methods and protocols for DNA sequencing and analysis are well-known in the art and are described herein. For example, DNA sequencing may be accomplished using high-throughput DNA sequencing techniques. Examples of next-generation and high-throughput sequencing include, for example, massively parallel signature sequencing, polony sequencing, 454 pyrosequencing, Illumina (Solexa) sequencing (e.g., HiSeq, MiSeq, other platforms), SOLiD sequencing, ion semiconductor sequencing (Ion Torrent), DNA nanoball sequencing, heliscope single molecule sequencing, single molecule real time (SMRT) sequencing, MassARRAY®, and Digital Analysis of Selected Regions (DANSR™). See, e.g., Stein (2008) Genetic Engineering & Biotechnology News 28(15); Quail et al. (2012) BMC Genomics 13(1):341; Liu et al. (2012) J of Biomedicine and Biotechnology 2012:1-11; Oeth et al. (2009) Methods Mol Biol. 578:307-43; Chu et al. (2010) Prenatal Diagnosis 30:1226-1229; and Suzuki et al. (2008) Clinica chimica acta international journal of clinical chemistry 387:55-8). Similarly, software programs for primary and secondary analysis of sequence data are well-known in the art.

Where there are multiple cell DNA samples from a recipient of allogeneic cells to be sequenced, such as when multiple samples are taken from the recipient of allogeneic cells over a specific time interval, each sample may be sequenced individually, or multiple samples may be sequenced together using multiplex sequencing.

### Analyzing Polymorphic Marker Allele Distribution Patterns and Determining the Levels of Allogeneic Cell DNA and Chimerism

The methods of the present disclosure involve assaying differences in polymorphic marker allele distribution patterns in a polymorphic marker panel as compared to expected homozygous or heterozygous distribution patterns. Analysis of these patterns allows for the level of allogeneic cell DNA and the level of chimerism in a sample obtained from a recipient of allogeneic cells to be determined. Without wishing to be bound by theory, it is assumed that the level of chimerism in a cell DNA sample directly correlates to the level of allogeneic cells in a sample. Thus, the level of chimerism in a cell DNA sample also directly relates to the level of allogeneic cell DNA present in the sample. For example, if analysis of a sample indicates that 95% of the DNA in a sample is allogeneic cell DNA, then the level of chimerism is 95%. In some embodiments, treatment of the recipient of allogeneic cells is adjusted based on the level of chimerism in the sample.

Generally, an individual contains DNA that is either homozygous or heterozygous for a given polymorphic marker, such as a SNP. An individual may be homozygous for one allele of a given polymorphic marker and will contain 100% of one allele of that polymorphic marker and 0% of the other allele of that polymorphic marker (e.g., 100% of allele A for a given polymorphic marker, 0% of allele B for a given polymorphic marker). An individual may also be heterozygous for a given polymorphic marker, and thus will contain 50% of allele A and 50% of allele B of that polymorphic marker. Accordingly, if all of the DNA in a sample originated from a single individual, it is expected that any given polymorphic marker in the DNA in that sample will exhibit a homozygous distribution pattern (i.e., 100% of one allele, 0% of the other allele) or a heterozygous distribution pattern (i.e., 50% of one allele and 50% of the other allele). However, if a DNA sample contains DNA that originated from more than one individual (e.g., a cell DNA from a recipient of allogeneic cells that contains both recipient-derived DNA and allogeneic cell DNA), then polymorphic marker allele distributions for a given polymorphic marker may be different than expected homozygous or heterozygous distribution patterns. This is so because two individuals may not necessarily share the same zygosity for a given polymorphic marker (e.g., individual 1 is homozygous for a given allele of a given polymorphic marker, and individual 2 is heterozygous for the alleles of that same polymorphic marker). When this occurs, differences in the expected allele distribution patterns as compared to expected homozygous or heterozygous distribution patterns may be observed. These differences can be used to assess whether foreign DNA is present in a DNA sample from a single individual. With respect to the methods of the present disclosure, differences in polymorphic marker allele distribution patterns in the polymorphic marker panel as compared to expected homozygous or heterozygous distribution patterns are used to determine the levels of allogeneic cell DNA and chimerism in the cell DNA sample obtained from a recipient of allogeneic cells.

When analyzing polymorphic marker sequence data from cell DNA according to the methods of the present disclosure, a majority signal from one allele of a polymorphic marker may be observed and a minority signal from another allele of a polymorphic marker may be observed. In some embodiments, it can be assumed that the majority of the DNA in the cell DNA sample from the recipient of allogeneic cells originated from the allogeneic cell DNA. Therefore, in these embodiments it can be further assumed that the majority signal represents an allele of a polymorphic marker that originated from allogeneic cell DNA, while the minority signal represents an allele of a polymorphic marker that originated from recipient-derived cell DNA. In some embodiments, it can be assumed that the majority of the DNA in the cell DNA sample from the recipient of allogeneic cells originated from the recipient. Therefore, in these embodiments it can be further assumed that the majority signal represents an allele of a polymorphic marker that originated from recipient-derived cell DNA, while the minority signal represents an allele of a polymorphic marker that originated from allogeneic cell DNA. Deviations from the expected allele frequency for a single individual (i.e., 0%, 50%, or 100%) will indicate the presence of allogeneic cell DNA and the level of chimerism.

Various calculations may be performed based on allele calls from the sequence data. For example, the methods of the present disclosure may involve calculating various cell DNA concentrations, or percentages thereof, of a total amount of cell DNA. This information can be used to calculate a percentage of allogeneic DNA in the cell DNA sample.

As described above, individual genotyping of the allogeneic cells and the recipient to determine which allele of the polymorphic marker belongs to the allogeneic cells and/or the recipient is not necessary, as differences in the polymorphic marker allele distribution pattern from expected homozygous or heterozygous distribution patterns inform the presence of allogeneic cell DNA in the population of cell DNA molecules isolated from the recipient of allogeneic cells. Accordingly, the level of allogeneic cell DNA or level of chimerism in a sample obtained from a recipient of allogeneic cells may be determined without using genotype information from the recipient of allogeneic cells, from the allogeneic cells, and/or any other genotype information from any source. Such genotype information that need not be considered includes, for example, the genotype across the whole genome or of portions thereof and/or the genotype at the particular polymorphic markers being analyzed. In some embodiments, individual genotyping of the recipient of allogeneic cells is not performed. In some embodiments, individual genotyping of the allogeneic cells is not performed. In some embodiments, neither the recipient of allogeneic cells nor the allogeneic cells are individually genotyped. In some embodiments, genotype information from the recipient of allogeneic cells is not considered when determining the levels of allogeneic cell DNA in a sample obtained from a recipient of allogeneic cells. In some embodiments, genotype information from the allogeneic cells is not considered when determining the levels of allogeneic cell DNA in a sample obtained from a recipient of allogeneic cells. In some embodiments, the levels of allogeneic cell DNA in a sample obtained from a recipient of allogeneic cells are determined without consideration of genotype information from the recipient of allogeneic cells and without consideration of genotype information of the allogeneic cells. In some embodiments, the level of allogeneic cell DNA in a sample obtained from a recipient of allogeneic cells may be determined without using any genotype information.

Improvements to the calculations for determining the level of allogeneic cell DNA or level of chimerism in a sample may include estimating and subtracting a level of signal due to amplification or sequencing error to improve accuracy and precision. For example, a suitably chosen subset of SNPs may be used to estimate a sum, mean, median, or standard deviation of the subset to produce a computation of the overall level of allogeneic cell DNA. Multiple samples from the same subject at the same time of sampling will all have the same pattern of polymorphic distributions across the SNPs, which can be used to enhance the estimate of allogeneic cell DNA in individual samples from that subject.

The quantity of allogeneic cell DNA present in the sample may be expressed in a variety of ways. In some embodiments, the amount of one or more DNA molecules from allogeneic cell DNA is determined as a percentage of the total the DNA molecules in the sample. In some embodiments, the amount of one or more DNA molecules from allogeneic cell DNA is determined as a ratio of the total DNA in the sample. In some embodiments, the amount of one or more DNA molecules from allogeneic cell DNA is determined as a ratio or percentage compared to one or more reference DNA molecules in the sample. For example, the total amount of allogeneic cell DNA can be determined to be 90% of the total DNA molecules in the cell DNA sample. Alternatively, the total amount of allogeneic cell DNA can be expressed as a ratio of 9:10 compared to the total DNA molecules in the cell DNA sample. In some embodiments, the amount of one or more DNA molecules from the allogeneic cell DNA can be determined as a concentration. For example, the total amount of allogeneic cell DNA in the cell DNA sample can be determined to be 1 μg/mL. The values described here are merely exemplary to illustrate various ways to express quantities of allogeneic cell DNA. In some cases, the percentage of allogeneic cell DNA in the cell DNA sample from a recipient of allogeneic cells may be extremely high (e.g., at or above 98% of the total DNA content of the cell DNA sample). It is noted that the quantity of recipient-derived cell DNA in the cell DNA sample may also be expressed in the manners as described for allogeneic cell DNA. Additional methods of expressing the quantity of a given source, type, or sequence of DNA molecule in a cell DNA sample will be readily apparent to one of skill in the art.

In some embodiments, the level of allogeneic cell DNA is used to calculate the percentage of allogeneic cells. In some embodiments, the level of allogeneic cell DNA is used to calculate the area under the curve (AUC) of the percentage of allogeneic cells over a time interval. In some embodiments, the level of allogeneic cell DNA is expressed as the percentage of allogeneic cells. In some embodiments, the level of cell DNA expressed as the area under the curve (AUC) of the percentage of allogeneic cells over a time interval

The level of chimerism present in the sample may be also expressed in a variety of ways. In some embodiments, the level of chimerism is determined as a percentage of allogeneic DNA in the sample. For example, the level of chimerism can be determined to be 90% when the level of allogeneic DNA in the sample is 90%. In some embodiments, the level of chimerism is determined as a ratio of allogeneic DNA in the sample to the total DNA in the sample. For example, the level of chimerism can be determined to be 9:10 when the ratio of allogeneic DNA to total DNA in a sample is 9:10. The values described here are merely exemplary to illustrate various ways to express levels of chimerism in a sample. In some cases, the level of chimerism in the sample from a recipient of allogeneic cells may be extremely high (e.g., at or above 98% chimerism). Additional methods of expressing the level of chimerism in a cell DNA sample will be readily apparent to one of skill in the art.

The above-described embodiments of the present disclosure may be implemented in a variety of ways. For example, some aspects of the embodiments may be implemented using hardware, software, or a combination thereof. In an exemplary embodiment, the software involves the conditional probability of Bayesian probability theorem, minimizes the use of prior distribution assumptions, and explicitly infers individual genotypes in the DNA mixture in a unique configuration to give a set of features that could not otherwise be achieved. In some embodiments, the software uses the conditional probability of Bayesian probability theorem P(A|B)=P(B|A)*P(A)/P(B) iteratively to calculate the allogeneic cell genomic fraction in the DNA mixture. In some embodiments, the software uses assumptions for the estimation based on Mendelian genetics and incorporates the biallelic and high population minor allele frequency features of the SNPs present in the assay. In some embodiments, the method estimates the allogeneic cell genomic fraction with these assumptions, and explicitly predicts the recipient and allogeneic cell genotypes based on the above estimation. In some embodiments, the method then refines the allogeneic cell genomic fraction given the predicted genotypes. In some embodiments, the method does not make any assumptions regarding the underlying statistical distribution of the sequenced data, for example Poisson, thus reducing the risk of introducing biases due to inappropriate model fitting. The design of this method is also flexible so that, in some embodiments, when recipient and/or allogeneic cell genotypes are known or can be individually measured, this genotype information can be used as input for a more accurate calculation.

When implemented in software, the software code can be executed on any suitable processor or collection of processors, whether provided in a single computer or distributed among multiple computers. It should be appreciated that any component or collection of components that perform the functions described above can be generically considered as one or more controllers that control the above discussed functions. The one or more controllers can be implemented in numerous ways, such as with dedicated hardware, or with general-purpose hardware (e.g., one or more processors) that is programmed using microcode or software to perform the functions recited above.

In this respect, it should be noted that implementation of various features of the present disclosure may use at least one non-transitory computer-readable storage medium (e.g., a USB drive, an external drive, computer memory, a compact disk, a floppy disk, a tape, etc.) encoded with a computer program (i.e., a plurality of instructions), which, when executed on a processor, performs the above discussed functions. The computer-readable storage medium can be transportable such that the program stored thereon can be loaded onto any computer resource to implement certain aspects of the present disclosure discussed herein. In addition, it should be noted that the reference to a computer program which, when executed, performs the above discussed functions, is not limited to an application program running on a host computer. Rather, the term computer program is used herein in a generic sense to reference any type of computer code (e.g., software or microcode) that can be employed to program a processor to implement certain aspects of the present disclosure.

### Determining the Status of Allogeneic Cells

The methods of the present disclosure involve determining the levels of allogeneic cell DNA or levels of chimerism in a sample from a recipient of allogeneic cells and can be used to determine the status of the allogeneic cells in the recipient of allogeneic cells.

In one aspect, the present disclosure provides a method of determining the status of allogeneic cells administered to a recipient. In some embodiments, the method of determining the status of allogeneic cells comprises a) administering the allogeneic cells to a recipient; b) providing cell DNA from a sample obtained from the recipient; c) sequencing a panel of single nucleotide polymorphisms (SNPs) from the cell DNA, wherein the panel of SNPs is suitable for differentiating between allogeneic cell DNA and recipient-derived cell DNA; and d) assaying differences in SNP allele distribution patterns in the panel as compared to expected homozygous or heterozygous distribution patterns to determine the level of allogeneic cell DNA. In some embodiments, a level of allogeneic cell DNA above a therapeutically effective threshold and/or increasing or stable over a time interval indicates a status of engraftment, expansion and/or persistence of the allogeneic cells. In some embodiments, a level of allogeneic cell DNA below a therapeutically effective threshold and/or decreasing over a time interval indicates a status of exhaustion, contraction, loss of persistence, allogeneic cell rejection, and/or graft vs. host disease. In some embodiments, allogeneic cell rejection is due to host vs. graft disease. In some embodiments, a level of allogeneic cell DNA below a therapeutically effective threshold and/or decreasing over a time interval indicates disease relapse. In some embodiments, a level of allogeneic cell DNA below a therapeutically effective threshold and/or decreasing over a time interval indicates a status of low quality allogeneic cells, e.g., allogeneic cells that are not viable, not capable of expanding, and/or not therapeutically effective. Further, the methods of determining the status of allogeneic cells may be used in other methods of the present disclosure, as described below. In some embodiments, determining the status of allogeneic cells may be informative with regard to a clinical decision involving the treatment of a recipient of allogeneic cells, for example, with respect to adjusting the treatment of the recipient.

In general, a level of allogeneic cell DNA above or below a therapeutic threshold, within or outside of a therapeutic range, and/or changes in the level of allogeneic cell DNA beyond a suitable threshold value in the recipient over a specific time interval are informative with regard to the status of the allogeneic cells. Similarly, a level chimerism above or below a therapeutic threshold, within or outside of a therapeutic range, and/or changes in the level of chimerism beyond a suitable threshold value in the recipient over a specific time interval are informative with regard to the status of the allogeneic cells. In some embodiments, the status of the allogeneic cells enables a clinical decision. In some embodiments, the status of the allogeneic cells is informative with regard to the adjustment of the treatment of a recipient of allogeneic cells.

A therapeutically effective threshold generally refers to any predetermined level or range that is indicative of the presence or absence of a condition or the presence or absence of a risk. For example, a level of allogeneic cell DNA or a level of chimerism below a therapeutically effective threshold may indicate allogeneic cell exhaustion, contraction, loss of persistence, allogeneic cell rejection, and/or graft vs. host disease. A level of allogeneic cell DNA or a level of chimerism below a therapeutically effective threshold may indicate disease relapse. A level of allogeneic cell DNA below a therapeutically effective threshold may indicate that the allogeneic cells are poor quality allogeneic cells, e.g., allogeneic cells that are not viable, not capable of expanding, and/or not therapeutically effective. As another example, a level of allogeneic cell DNA above a therapeutically effective threshold and/or increasing or stable over a time interval may indicate allogeneic cell engraftment, expansion and/or persistence. The therapeutically effective threshold value can take a variety of forms. In some embodiments, the therapeutically effective threshold is a single cut-off value, such as a median or mean. As another example, a threshold value can be determined from baseline values before the presence of a condition or risk, and/or after a course of treatment. Such a baseline can be indicative of a normal or other state in the recipient not correlated with the risk or condition that is being tested for. For example, the baseline value may be the level of allogeneic cell DNA or level of chimerism in samples from a recipient of allogeneic cells prior to the actual allogeneic cell administration event, which would be presumably zero or negligible, but may also indicate baseline error in the system. In some embodiments, the threshold value can be a baseline value of the recipient being tested. The threshold value, as it pertains to demarcating significant changes in the levels of allogeneic cell DNA or chimerism in a sample, may vary considerably. In some embodiments, the therapeutically effective threshold of allogeneic cell DNA or chimerism is 0.001%, 0.01%, 0.02%, 0.03%, 0.04%, 0.05%, 0.06%, 0.07%, 0.08%, 0.09%, 0.1%, 0.2%, 0.3%, 0.4%, 0.5%, 0.6%, 0.7%, 0.8%, 0.9%, 1.0%, 1.1%, 1.2%, 1.3%, 1.4%, 1.5%, 1.6%, 1.7%, 1.8%, 1.9%, 2.0%, 2.1%, 2.2%, 2.3%, 2.4%, 2.5%, 2.6%, 2.7%, 2.8%, 2.9%, 3.0%, 3.1%, 3.2%, 3.3%, 3.4%, 3.5%, 3.6%, 3.7%, 3.8%, 3.9%, 4.0%, 4.1%, 4.2%, 4.3%, 4.4%, 4.5%, 4.6%, 4.7%, 4.8%, 4.9%, 5.0%, 5.1%, 5.2%, 5.3%, 5.4%, 5.5%, 5.6%, 5.7%, 5.8%, 5.9%, 6.0%, 6.1%, 6.2%, 6.3%, 6.4%, 6.5%, 6.6%, 6.7%, 6.8%, 6.9%, 7.0%, 7.1%, 7.2%, 7.3%, 7.4%, 7.5%, 7.6%, 7.7%, 7.8%, 7.9%, 8.0%, 8.1%, 8.2%, 8.3%, 8.4%, 8.5%, 8.6%, 8.7%, 8.8%, 8.9%, 9.0%, 9.1%, 9.2%, 9.3%, 9.4%, 9.5%, 9.6%, 9.7%, 9.8%, 9.9%, 10.0%, 10.1%, 10.2%, 10.3%, 10.4%, 10.5%, 10.6%, 10.7%, 10.8%, 10.9%, 11.0%, 11.1%, 11.2%, 11.3%, 11.4%, 11.5%, 11.6%, 11.7%, 11.8%, 11.9%, 12.0%, 12.1%, 12.2%, 12.3%, 12.4%, 12.5%, 12.6%, 12.7%, 12.8%, 12.9%, 13.0%, 13.1%, 13.2%, 13.3%, 13.4%, 13.5%, 13.6%, 13.7%, 13.8%, 13.9%, 14.0%, 14.1%, 14.2%, 14.3%, 14.4%, 14.5%, 14.6%, 14.7%, 14.8%, 14.9%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 90.1%, 90.2%, 90.3%, 90.4%, 90.5%, 90.6%, 90.7%, 90.8%, 90.9%, 91.0%, 91.1%, 91.2%, 91.3%, 91.4%, 91.5%, 91.6%, 91.7%, 91.8%, 91.9%, 92.0%, 92.1%, 92.2%, 92.3%, 92.4%, 92.5%, 92.6%, 92.7%, 92.8%, 92.9%, 93.0%, 93.1%, 93.2%, 93.3%, 93.4%, 93.5%, 93.6%, 93.7%, 93.8%, 93.9%, 94.0%, 94.1%, 94.2%, 94.3%, 94.4%, 94.5%, 94.6%, 94.7%, 94.8%, 94.9%, 95.0%, 95.1%, 95.2%, 95.3%, 95.4%, 95.5%, 95.6%, 95.7%, 95.8%, 95.9%, 96.0%, 96.1%, 96.2%, 96.3%, 96.4%, 96.5%, 96.6%, 96.7%, 96.8%, 96.9%, 97.0%, 97.1%, 97.2%, 97.3%, 97.4%, 97.5%, 97.6%, 97.7%, 97.8%, 97.9%, 98.0%, 98.1%, 98.2%, 98.3%, 98.4%, 98.5%, 98.6%, 98.7%, 98.8%, 98.9%, 99.0%, 99.1%, 99.2%, 99.3%, 99.4%, 99.5%, 99.6%, 99.7%, 99.8%, 99.9%, or 100%.

One of skill in the art would recognize appropriate parameters and means for determining significant changes in the levels of allogeneic cell DNA or chimerism in a recipient of allogeneic cells over a specific time interval and would readily understand when the level of allogeneic DNA or level of chimerism was increasing, decreasing, or remaining stable over a time interval. Once appropriate analysis parameters are selected, determining changes in the level of allogeneic cell DNA or level of chimerism in the recipient of allogeneic cells over a time interval can inform the status of the allogeneic cells.

In some embodiments, a level of allogeneic cell DNA that is below a therapeutically effective threshold at a point in time and/or that is decreasing over a specific time interval is indicative of allogeneic cell exhaustion, allogeneic cell contraction, loss of allogeneic cell persistence, allogeneic cell rejection, a need for adjusting therapy or treatment of the recipient of allogeneic cells, immunosuppressive treatment nephrotoxicity, disease relapse, and/or a need for further investigation of the allogeneic cell status. In some embodiments, a level of allogeneic cell DNA that is below a therapeutically effective threshold at a point in time and/or that is decreasing over a specific time interval is indicative of allogeneic cell rejection. In some embodiments, a level of allogeneic cell DNA that is below a therapeutically effective threshold at a point in time and/or that is decreasing over a specific time interval is indicative of allogeneic cell exhaustion. In some embodiments, a level of allogeneic cell DNA that is below a therapeutically effective threshold at a point in time and/or that is decreasing over a specific time interval is indicative of allogeneic cell contraction. In some embodiments, a level of allogeneic cell DNA below a therapeutically effective threshold and/or decreasing over a time interval indicates that the allogeneic cells are poor quality allogeneic cells, e.g., allogeneic cells that are not viable, not capable of expanding, and/or not therapeutically effective. Without wishing to be bound by theory, it is thought that allogeneic cell rejection is associated with the death and/or lack of replication of allogeneic cells, resulting in a level of allogeneic cells that is lower than a therapeutically effective level or threshold and/or resulting in a decreasing level of allogeneic cells over a specific time interval.

In some embodiments, a level of allogeneic cell DNA that is above a therapeutically effective threshold at a point in time and/or that is increasing or remaining stable over a specific time interval is indicative of allogeneic cell engraftment, allogeneic cell expansion, allogeneic cell persistence, allogeneic cell tolerance, a need for adjusting therapy or treatment of the recipient of allogeneic cells, and/or a need for further investigation of the allogeneic cell status. In some embodiments, a level of allogeneic cell DNA that is above a therapeutically effective threshold at a point in time is indicative of allogeneic cell engraftment, expansion and/or persistence. In some embodiments, a level of allogeneic cell DNA that is increasing over a specific time interval is indicative of allogeneic cell engraftment, expansion and/or persistence. In some embodiments, a level of allogeneic cell DNA that is above a therapeutically effective threshold and that is increasing over a specific time interval is indicative of allogeneic cell engraftment, expansion and/or persistence. In some embodiments, a level of allogeneic cell DNA that is above a therapeutically effective threshold and that is remaining stable over a specific time interval is indicative of allogeneic cell engraftment, expansion and/or persistence. In some embodiments, a level of allogeneic cell DNA that is above a therapeutically effective threshold at a point in time and that is increasing or remaining stable over a specific time interval is indicative of allogeneic cell persistence. Without wishing to be bound by theory, it is thought that allogeneic cell engraftment, expansion and/or persistence is associated with the ability of allogeneic cells to survive and/or replicate in the recipient, resulting in a level of allogeneic cells that is at or higher than a therapeutically effective level or threshold and/or resulting in an increasing or stable level of allogeneic cells over a specific time interval. Without wishing to be bound by theory, it is also believed that if the level of allogeneic cell DNA is increasing in a recipient of allogeneic cells over a specific time interval, then the allogeneic cells are decreasingly experiencing apoptosis and/or necrosis over a specific time interval, which is indicative of allogeneic cell engraftment, allogeneic cell expansion, allogeneic cell persistence, allogeneic cell tolerance, overimmunosuppression, or appropriate immunosuppression.

### Guiding Treatment, Informing, Providing Information

The methods of the present disclosure for determining the status of allogeneic cells in a recipient of allogeneic cells can be used to provide information and/or guide treatment of the recipient of allogeneic cells.

In general, identifying a status of engraftment, expansion and/or persistence of the allogeneic cells, and a level of allogeneic cell DNA below a therapeutically effective threshold and/or decreasing over a time interval as described herein is informative with regard to a need to guide treatment of the recipient of allogeneic cells. Accordingly, in some embodiments, the methods of determining the status of allogeneic cells further comprise guiding treatment of the recipient of the allogeneic cells based on the status of the allogeneic cells.

In some embodiments, the methods of determining the status of allogeneic cells further comprise providing information on the status of the allogeneic cells. Information on the status of the allogeneic cells may be provided in the form of a report summarizing the status of the allogeneic cells.

In some embodiments, the methods of determining the status of allogeneic cells further comprise providing information on a need to guide treatment of the recipient of the allogeneic cells. For example, determining the status of the allogeneic cells may be informative with respect to a need to adjust treatment of the recipient of allogeneic cells, treat allogeneic cell rejection, or monitor for relapse of a hematologic cancer, as described in detail below. Information on a need to guide treatment of the recipient of the allogeneic cells may be provided in the form of a report summarizing the status of the allogeneic cells and providing guidance on options for treating the recipient of the allogeneic cells.

### Adjusting Treatment of Recipient of Allogeneic Cells

The methods of the present disclosure for determining the levels of allogeneic cell DNA in a sample from a recipient of allogeneic cells can be used to inform the need to adjust therapy being administered to the recipient of allogeneic cells. In general, a level of allogeneic cell DNA above or below a therapeutic threshold and/or changes in the level of allogeneic cell DNA beyond a suitable threshold value in the recipient over a specific time interval are informative with regard to determining a need to adjust therapy being administered to the recipient of allogeneic cells. In some embodiments, determining the status of the allogeneic cells, as described above, is informative with regard to determining a need to adjust therapy being administered to the recipient of allogeneic cells. In some embodiments, determining the status of the allogeneic cells is informative with regard to determining the way in which therapy should be adjusted.

In one aspect of the present disclosure, a method of administering allogeneic cells to a recipient and adjusting treatment of the recipient is provided. In some embodiments, the method comprises a) administering the allogeneic cells to a recipient; b) providing cell DNA from a sample obtained from the recipient; c) sequencing a panel of single nucleotide polymorphisms (SNPs) from the cell DNA, wherein the panel of SNPs is suitable for differentiating between allogeneic cell DNA and recipient-derived cell DNA; d) assaying differences in SNP allele distribution patterns in the panel as compared to expected homozygous or heterozygous distribution patterns to determine the level of allogeneic cell DNA; and e) adjusting treatment of the recipient of the allogeneic cells based on the status of the allogeneic cells. The status of the allogeneic cells may be determined as described above. For example, in some embodiments, a level of allogeneic cell DNA above a therapeutically effective threshold and/or increasing or stable over a time interval indicates a status of engraftment, expansion and/or persistence of the allogeneic cells. In some embodiments, a level of allogeneic cell DNA below a therapeutically effective threshold and/or decreasing over a time interval indicates a status of exhaustion, contraction, loss of persistence, allogeneic cell rejection, and/or graft vs. host disease. In some embodiments, allogeneic cell rejection is due to host vs. graft disease. In some embodiments, a level of allogeneic cell DNA below a therapeutically effective threshold and/or decreasing over a time interval indicates disease relapse. In some embodiments, a level of allogeneic cell DNA below a therapeutically effective threshold and/or decreasing over a time interval indicates a status of low quality allogeneic cells, e.g., allogeneic cells that are not viable, not capable of expanding, and/or not therapeutically effective. In some alternative embodiments, instead of a step of adjusting immunosuppressive therapy, the method comprises providing information on a need to adjust treatment. In some alternative embodiments, instead of a step of adjusting immunosuppressive therapy, the method comprises having treatment adjusted as described herein.

It is to be understood that therapies of the present disclosure may be administered in a number of ways, including intravenously, intratumorally, intramuscularly, subcutaneously, intrathecally, or orally. The route of therapy will depend on the type of therapy that is being administered. Appropriate routes of therapy for allogeneic cells, immunosuppressive therapy, and other allogeneic cell-related therapies will be evident to one of skill in the art.

### Adjusting Allogeneic Cell Therapy

The methods of the present disclosure for determining the levels of allogeneic cell DNA in a sample from a recipient of allogeneic cells can be used to inform the need to adjust allogeneic cell therapy being administered to the recipient of allogeneic cells. In general, a level of allogeneic cell DNA above or below a therapeutic threshold and/or changes in the level of allogeneic cell DNA beyond a suitable threshold value in the recipient over a specific time interval are informative with regard to determining a need to adjust allogeneic cell therapy being administered to the recipient of allogeneic cells. In some alternative embodiments, instead of adjusting allogeneic cell therapy, the method comprises providing information on a need to adjust allogeneic cell therapy. In some alternative embodiments, instead of adjusting allogeneic cell therapy, the method comprises having allogeneic cell therapy adjusted.

Allogeneic cell therapy generally refers to the administration of allogeneic cells to a recipient. Exemplary allogeneic cells may include, for example, blood cells (e.g., hematopoietic stem cells, peripheral blood mononuclear cells, T cells, T reg cells, B cells, CAR T cells, NK cells, NKT cells, TILs), stem cells (e.g., embryonic, tissue-specific, mesenchymal, induced pluripotent, hematopoietic, skeletal, myogenic, cardiac, neural, epidermal, or intestinal stem cells), cardiomyocytes, neurons, lymphocytes, macrophages, dendritic cells, and pancreatic islet cells. In some embodiments, the allogeneic cells are blood cells. In some embodiments, the allogeneic cells are T cells. In some embodiments, the allogeneic T cells are chimeric antigen receptor (CAR) T cells, universal CAR T cells (i.e., where the CAR binds to an antibody that binds a specific antigen), split CAR T cells (i.e., where a dimerizing agent activates CAR T cell function), activatable CAR T cells, repressible CAR T cells, multiphasic CAR T cells (i.e., where the CAR must bind multiple specific antigens and/or agents to induce T cell activation), tumor infiltrating lymphocytes, regulatory T cells, genetically modified T cells, T cells with genetically modified or synthesized T cell receptors (TCRs), or virus-specific T cells (e.g., EBV, HPV, BKV, CMV, etc.).

Allogeneic cell therapy may involve one or more, two or more, three or more, four or more, five or more, six or more, seven or more, eight or more, nine or more, ten or more, fifteen or more, or twenty or more separate treatments with allogeneic cells. In some embodiments, the planned number of allogeneic cell treatments is increased, decreased, or maintained based on the status of the allogeneic cells or level of chimerism in the recipient.

Allogeneic cell therapy may be given at particular dosages per treatment. In some embodiments, 1×10⁵ cells, 1×10⁶ cells, 1×10⁷ cells, 1×10⁸ cells, or 1×10⁹ cells, or any value in between, are given to the recipient in one administration.

### Adjusting in Response to a Level of Allogeneic Cell DNA Below a Therapeutic Threshold or a Decrease in the Level of Allogeneic Cell DNA in the Recipient of Allogeneic Cells Over a Time Interval

In some embodiments, a level of allogeneic cell DNA below a therapeutic threshold or a decrease in the level of allogeneic cell DNA in the recipient of allogeneic cells over a specific time interval is indicative of a need to continue, adjust, or increase administration of the allogeneic cell therapy being administered to the recipient of allogeneic cells. In some embodiments where the level of allogeneic cell DNA is below a therapeutically effective threshold and/or decreasing over a specific time interval, allogeneic cell therapy being administered to the recipient is adjusted by continuing administration of the allogeneic cells originally administered to the recipient. In some embodiments, allogeneic cell therapy being administered to the recipient is adjusted by administering allogeneic cells that are different than those originally administered to the recipient. In some embodiments, allogeneic cell therapy being administered to the recipient is adjusted by continuing administration of the allogeneic cells originally administered to the recipient and administering allogeneic cells that are different than those originally administered to the recipient.

Continuing administration of the allogeneic cells may include administration of the same dosage, the same dosage rate (i.e., cells per unit of time), a higher dosage, and/or a higher dosage rate of allogeneic cells to the recipient compared to the dosage or dosage rate of allogeneic cells originally administered to the recipient. In addition, continuing administration can be performed over the same amount of time, a longer amount of time, or a shorter amount of time compared to the original allogeneic cell administration time, as long as the total dosage and/or dosage rate is the same or increased compared to the dosage or dosage rate of allogeneic cells originally administered to the recipient. In some embodiments, continuing administration of the allogeneic cells involves administering the same total dosage of allogeneic cells to the recipient for a shorter period of time (i.e., a higher dosage rate). In some embodiments, continuing administration of the allogeneic cells involves administering a higher dosage of allogeneic cells to the recipient for the same amount of time. In some embodiments, continuing administration of the allogeneic cells involves administering a higher dosage of allogeneic cells to the recipient for a longer time interval.

In some embodiments in which allogeneic cells of a different type are administered to the recipient compared to those originally administered to the recipient, the dosage, dosage rate, and timing of allogeneic cell administration may be the same or may vary compared to the originally administered allogeneic cells.

In some embodiments where the level of allogeneic cell DNA is below a therapeutically effective threshold and/or decreasing over a specific time interval, allogeneic cell therapy being administered to the recipient is adjusted by administering an increased dose of the allogeneic cells. In some embodiments, the increased dose of cells is a higher volume and/or concentration of cells than the dose of allogeneic cells originally administered to the recipient.

In some embodiments where the level of allogeneic cell DNA is below a therapeutically effective threshold and/or decreasing over a specific time interval, allogeneic cell therapy being administered to the recipient is adjusted by administering doses of the allogeneic cells more frequently. In some embodiments, doses of allogeneic cells are administered more frequently than the dose of allogeneic cells originally administered to the recipient.

Initiating, continuing, adjusting, or increasing allogeneic cell treatment may be combined with the initiation, continuation, discontinuation, increase, or decrease of a different allogeneic cell therapy, which may include any number of cell therapies known in the art, such as the allogeneic cell therapies described above.

Initiating, continuing, adjusting, or increasing the dose or frequency of allogeneic cell administration can also be combined with adjusting other therapies or treatments, such as immunosuppressive therapy. In some embodiments where the level of allogeneic cell DNA is below a therapeutically effective threshold and/or decreasing over a specific time interval, treatment of the recipient is adjusted by continuing administration of the allogeneic cells originally administered to the recipient, administering allogeneic cells that are different than those originally administered to the recipient, initiating immunosuppressive therapy, adjusting immunosuppressive therapy, or a combination thereof.

### Adjusting in Response to a Level of Allogeneic Cell DNA Above a Therapeutic Threshold or an Increase in the Level of Allogeneic Cell DNA in the Recipient of Allogeneic Cells Over a Time Interval

In some embodiments, a level of allogeneic cell DNA above a therapeutic threshold or an increase in the level of allogeneic cell DNA in the recipient of allogeneic cells over a specific time interval is indicative of a need to adjust, reduce, or discontinue administration of the allogeneic cell therapy being administered to the recipient of allogeneic cells. In some embodiments where the level of allogeneic cell DNA is above a therapeutically effective threshold and/or increasing or stable over a specific time interval, allogeneic cell therapy being administered to the recipient is adjusted, reduced, or discontinued. In some embodiments where the level of allogeneic cell DNA is above a therapeutically effective threshold and/or increasing or stable over a specific time interval, allogeneic cell therapy being administered to the recipient is administered at a reduced dose of the allogeneic cells. In some embodiments where the level of allogeneic cell DNA is above a therapeutically effective threshold and/or increasing or stable over a specific time interval, the recipient is administered doses of the allogeneic cells less frequently. In some embodiments where the level of allogeneic cell DNA is above a therapeutically effective threshold and/or increasing or stable over a specific time interval, allogeneic cell therapy being administered to the recipient is discontinued. In some embodiments where the level of allogeneic cell DNA is above a therapeutically effective threshold and/or increasing or stable over a specific time interval, allogeneic cells are administered that are different than those originally administered.

Reducing allogeneic cell therapy may be performed over a variety of time intervals, dosages, and/or dosage rates (i.e., cells per unit of time), as long as the total dosage and/or dosage rate is lower than the dosage or dosage rate of the allogeneic cells originally administered to the recipient. In some embodiments, reducing allogeneic cell therapy may involve administration of the same dosage, the same dosage rate (i.e., cells per unit of time), a lower dosage, and/or a lower dosage rate of allogeneic cells to the recipient compared to the dosage or dosage rate of allogeneic cells originally administered to the recipient. In some embodiments, reducing allogeneic cell therapy may be performed over the same amount of time, a longer amount of time, or a shorter amount of time compared to the administration time of the allogeneic cells originally given to the recipient. In some embodiments, reducing allogeneic cell therapy involves administering the same total dosage of allogeneic cells to the recipient for a longer period of time (i.e., a lower dosage rate). In some embodiments, reducing allogeneic cell therapy involves administering a lower dosage of allogeneic cells to the recipient for the same amount of time. In some embodiments, reducing allogeneic cell therapy involves administering a lower dosage of allogeneic cells to the recipient for a shorter period of time. In some embodiments, reducing allogeneic cell therapy involves administering reducing doses of allogeneic cells less frequently. In some embodiments, reducing allogeneic cell therapy involves administering a reduced dose (e.g., at a lower concentration and/or volume) of allogeneic cells than was originally administered to the recipient.

Adjusting, reducing, or discontinuing allogeneic cell treatment may be combined with the initiation, continuation, discontinuation, increase, or decrease of a different allogeneic cell therapy, which may include any number of cell therapies known in the art, such as the allogeneic cell therapies described above.

Adjusting, reducing, or discontinuing allogeneic cell therapy may also be combined with adjusting other therapies or treatments, such as immunosuppressive therapy. In some embodiments where the level of allogeneic cell DNA is above a therapeutically effective threshold and/or increasing or stable over a specific time interval, treatment of the recipient is adjusted by administering a reduced dose of the same allogeneic cells originally administered to the recipient, discontinuing administration of the allogeneic cells originally administered to the recipient, reducing immunosuppressive therapy, discontinuing immunosuppressive therapy, or a combination thereof.

### Adjusting Immunosuppressive Therapy

The methods of the present disclosure for determining the levels of allogeneic cell DNA in a sample from a recipient of allogeneic cells can be used to inform the need to adjust immunosuppressive therapy being administered to the recipient of allogeneic cells. In general, a level of allogeneic cell DNA above or below a therapeutic threshold and/or changes in the level of allogeneic cell DNA beyond a suitable threshold value in the recipient over a specific time interval are informative with regard to determining a need to adjust immunosuppressive therapy being administered to the recipient of allogeneic cells. In some alternative embodiments, instead of adjusting immunosuppressive therapy, the method comprises providing information on a need to adjust immunosuppressive therapy. In some alternative embodiments, instead of adjusting immunosuppressive therapy, the method comprises having immunosuppressive therapy adjusted.

Immunosuppressive therapy generally refers to the administration of an immunosuppressant or other therapeutic agent that suppresses immune responses to a subject. Exemplary immunosuppressant agents may include, but are not limited to, ACE inhibitors, anticoagulants, antimalarials, β-blockers, corticosteroids, cardiovascular drugs, non-steroidal anti-inflammatory drugs (NSAIDs), and steroids including, for example, aspirin, azathioprine, B7RP-1-fc, brequinar sodium, campath-1H, celecoxib, chloroquine, coumadin, cyclophosphamide, cyclosporin A, DHEA, deoxyspergualin, dexamethasone, diclofenac, dolobid, etodolac, everolimus, FK778, feldene, fenoprofen, flurbiprofen, heparin, hydralazine, hydroxychloroquine, CTLA-4 or LFA3 immunoglobulin, ibuprofen, indomethacin, ISAtx-247, ketoprofen, ketorolac, leflunomide, meclophenamate, mefenamic acid, mepacrine, 6-mercaptopurine, meloxicam, methotrexate, mizoribine, mycophenolate mofetil, naproxen, oxaprozin, Plaquenil, NOX-100, prednisone, methylprednisolone, rapamycin (sirolimus), sulindac, tacrolimus (FK506), thymoglobulin, tolmetin, tresperimus, UO126, as well as antibodies including, for example, alpha lymphocyte antibodies, adalimumab, anti-CD3, anti-CD25, anti-CD52 anti-IL2R, anti-TAC antibodies, basiliximab, daclizumab, etanercept, hu5C8, infliximab, OKT4, and natalizumab.

### Adjusting in Response to a Level of Allogeneic Cell DNA Below a Therapeutic Threshold or a Decrease in the Level of Allogeneic Cell DNA in the Recipient of Allogeneic Cells Over a Time Interval

In some embodiments, a level of allogeneic cell DNA below a therapeutic threshold or a decrease in the level of allogeneic cell DNA in the recipient of allogeneic cells over a specific time interval is indicative of a need to initiate, adjust, continue, or increase administration of an immunosuppressive therapy being administered to the recipient of allogeneic cells. In some embodiments where the level of allogeneic cell DNA is below a therapeutically effective threshold and/or decreasing over a specific time interval, immunosuppressive therapy being administered to the recipient is initiated, adjusted, continued, or increased.

Adjusting, continuing, or increasing administration of the immunosuppressive therapy may be performed over a variety of time intervals, dosages, and/or dosage rates (i.e., cells per unit of time). In some embodiments, adjusting, continuing, or increasing immunosuppressive therapy may involve administration of the same dosage, the same dosage rate, a higher dosage, and/or a higher dosage rate compared to the dosage or dosage rate of the immunosuppressive therapy previously administered to the recipient. In some embodiments, continuing administration of the immunosuppressive therapy involves administering the same total dosage of immunosuppressive therapy to the recipient for the same amount of time. In some embodiments, continuing administration of the immunosuppressive therapy involves administering the same total dosage of immunosuppressive therapy to the recipient for a shorter period of time (i.e., a higher dosage rate). In some embodiments, continuing administration of the immunosuppressive therapy involves administering a higher dosage of immunosuppressive therapy to the recipient for the same amount of time. In some embodiments, continuing administration of the immunosuppressive therapy involves administering a higher dosage of immunosuppressive therapy to the recipient for a longer period of time.

Increasing administration of the immunosuppressive therapy may be performed over a variety of time intervals, dosages, and/or dosage rates, as long as the total dosage and/or dosage rate is increased compared to the dosage or dosage rate of immunosuppressive therapy previously administered to the recipient. In some embodiments, increasing administration of the immunosuppressive therapy involves administering the same total dosage of immunosuppressive therapy to the recipient for a shorter period of time (i.e., a higher dosage rate). In some embodiments, increasing administration of the immunosuppressive therapy involves administering a higher dosage of immunosuppressive therapy to the recipient for the same amount of time. In some embodiments, increasing administration of the immunosuppressive therapy involves administering a higher dosage of immunosuppressive therapy to the recipient for a longer period of time. In some embodiments where the recipient of allogeneic cells is not receiving immunosuppressive therapy, the methods of the present disclosure may indicate a need to begin administering immunosuppressive therapy to the recipient of allogeneic cells.

Initiating, adjusting, continuing, or increasing immunosuppressive therapy can be combined with adjusting other therapies or treatments, such as allogeneic cell therapy. In some embodiments where the level of allogeneic cell DNA is below a therapeutically effective threshold and/or decreasing over a specific time interval, treatment of the recipient is adjusted by continuing administration of the allogeneic cells originally administered to the recipient, administering allogeneic cells that are different than those originally administered to the recipient, initiating immunosuppressive therapy, adjusting immunosuppressive therapy, or a combination thereof.

### Adjusting in Response to a Level of Allogeneic Cell DNA Above a Therapeutic Threshold or an Increase in the Level of Allogeneic Cell DNA in the Recipient of Allogeneic Cells Over a Time Interval

In some embodiments, a level of allogeneic cell DNA above a therapeutic threshold or an increase in the level of allogeneic cell DNA in the recipient of allogeneic cells over a specific time interval is indicative of a need to adjust, reduce, or discontinue immunosuppressive therapy being administered to the recipient of allogeneic cells. In some embodiments where the level of allogeneic cell DNA is above a therapeutically effective threshold and/or increasing or stable over a specific time interval, immunosuppressive therapy being administered to the recipient is adjusted, reduced, or discontinued.

Reducing administration of the immunosuppressive therapy may be performed over a variety of time intervals, dosages, and/or dosage rates (i.e., cells per unit of time), as long as the total dosage and/or dosage rate is reduced compared to the dosage or dosage rate of immunosuppressive therapy previously administered to the recipient. In some embodiments, reducing immunosuppressive therapy may involve administering the same total dosage of immunosuppressive therapy to the recipient for a longer period of time (i.e., a lower dosage rate). In some embodiments, reducing immunosuppressive therapy may involve administering a lower dosage of immunosuppressive therapy to the recipient for the same amount of time. In some embodiments, reducing immunosuppressive therapy may involve administering a lower dosage of immunosuppressive therapy to the recipient for a shorter period of time. In some embodiments where the recipient of allogeneic cells is receiving immunosuppressive therapy, the methods of the present disclosure may indicate a need to discontinue immunosuppressive therapy in the recipient of allogeneic cells.

Adjusting, reducing, or discontinuing immunosuppressive therapy can be combined with adjusting other therapies or treatments, such as allogeneic cell treatment. In some embodiments where the level of allogeneic cell DNA is above a therapeutically effective threshold and/or increasing or stable over a specific time interval, treatment of the recipient is adjusted by reducing the dosage of the allogeneic cells originally administered to the recipient, discontinuing administration of the allogeneic cells originally administered to the recipient, reducing immunosuppressive therapy, discontinuing immunosuppressive therapy, or a combination thereof.

In some embodiments where immunosuppressive therapy was previously administered to the recipient of allogeneic cells, adjustment of immunosuppressive therapy includes changing the type or form of the immunosuppressive therapy being administered to the recipient of allogeneic cells. The new type or form of immunosuppressive therapy may include any number of immunosuppressive therapies known in the art, such as the immunosuppressive therapies described above. Furthermore, the dosage, dosage rate, and timing of immunosuppressive therapy administration may or may not vary compared to those of the previously administered immunosuppressive therapy.

### Adjusting Other Allogeneic Cell-Related Therapies

The methods of the present disclosure for determining the levels of allogeneic cell DNA in a sample from a recipient of allogeneic cells can be used to inform the need to adjust other allogeneic cell-related therapies being administered to the recipient of allogeneic cells. In general, a level of allogeneic cell DNA above or below a therapeutic threshold and/or changes in the level of allogeneic cell DNA beyond a suitable threshold value in the recipient over a specific time interval are informative with regard to determining a need to adjust other allogeneic cell-related therapies being administered to the recipient of allogeneic cells. In some alternative embodiments, instead of adjusting other allogeneic cell-relater therapies, the method comprises providing information on a need to adjust other allogeneic cell-related therapies. In some alternative embodiments, instead of adjusting other allogeneic cell-relater therapies, the method comprises having other allogeneic cell-related therapies adjusted.

Other allogeneic cell-related therapies include treatments or therapies besides allogeneic cell therapy or immunosuppressive therapy that are administered to a recipient of allogeneic cells to affect the allogeneic cells (e.g., activate, inhibit, kill, or promote survival of allogeneic cells) or to treat allogeneic cell-related symptoms (e.g., cytokine release syndrome, neurotoxicity). Examples of other allogeneic cell-related therapies include but are not limited to administration of antibodies, antigen-targeting ligands, non-immunosuppressive drugs, and other agents that stabilize or destabilize components of allogeneic cells that are critical to one or more allogeneic cell activity or that directly activate or inhibit one or more allogeneic cell activity. These activities may include the ability to stably express particular receptors (e.g., CARs), induce an immune response, recognize particular antigens, replicate, and/or induce repair of damaged tissues. Adjusting allogeneic cell=therapy and/or immunosuppressive therapy may be combined with adjusting, initiating, or discontinuing other allogeneic cell-related therapies.

In some embodiments where the allogeneic cells are CAR T cells, adjusting treatment of the allogeneic cells involves initiating administration of a CAR T cell regulatory agent or adjusting the dosage of a CAR T cell regulatory agent administered to the recipient. CAR T cell regulatory agents generally activate or deactivate at least one activity of CAR T cells or alternatively promote survival, replication, or cell death of CAR T cells. For example, CAR T cells may be genetically modified to express a molecular switch which induces activation of the CAR T cells under particular conditions (e.g., upon addition or removal of a drug or antibody). This strategy may be used to generate CAR T cells with molecular switches that, for example, induce or repress the expression of the CAR expressed on the CAR T cell, or stabilize or destabilize the CAR expressed on the CAR T cell. CAR T cell regulatory agents may work directly on the CAR T cell, for example, by binding the CAR or by binding another expressed protein on the cell to promote CAR T cell survival, induce CAR T cell death, activate CAR T cell function, or inhibit CAR T cell function. Alternatively, CAR T cell regulatory agents (e.g., antibodies that bind both universal CAR T cells and specific tumor antigens) may work indirectly on the CAR T cell, for example, by acting as a bridge between the target cell and the CAR T cell in order to activate the CAR T cell. In some embodiments, the CAR T cell regulatory agent is a polynucleotide, polypeptide, small molecule drug, or antibody. Exemplary CAR T cell regulatory agents include caspase-9 activators (e.g., rimiducid), rapamycin (i.e., sirolimus), rapalogs (e.g., AP21967, ridaforolimus (i.e., deforolimus), everolimus, temsirolimus), antibodies (e.g., cancer targeting antibodies, antibodies that bind universal CAR T cells), CAR expression inhibitors (e.g., miRNA, siRNA, epigenetic modifiers, transcription or translation inhibitors, proteases), veledimex, ultrasound, and any other agent that specifically inhibits, activates, kills, and/or promotes the proliferation of CAR T cells. In some embodiments, the CAR T cell regulatory agent is an immune checkpoint inhibitor (e.g., a PD-1 inhibitor, a PD-L1 inhibitor, or a CTLA-4 inhibitor).

Methods of the present disclosure that involve adjusting the dosage of a CAR T cell regulatory agent may be performed over a variety of time intervals, dosages, and/or dosage rates (i.e., cells per unit of time), as long as the total dosage and/or dosage rate is different than that of the previously administered CAR T cell regulatory agent. In some embodiments, adjusting administration of the CAR T cell regulatory agent involves administering the same total dosage of CAR T cell regulatory agent to the recipient for a shorter amount of time (i.e., a higher dosage rate). In some embodiments, continuing administration of the CAR T cell regulatory agent involves administering a higher dosage of CAR T cell regulatory agent to the recipient for the same amount of time. In some embodiments, adjusting administration of the CAR T cell regulatory agent involves administering the same total dosage of CAR T cell regulatory agent to the recipient for a longer amount of time (i.e., a lower dosage rate). In some embodiments, continuing administration of the CAR T cell regulatory agent involves administering a lower dosage of CAR T cell regulatory agent to the recipient for the same amount of time.

In some embodiments where the recipient of allogeneic cells is not receiving a CAR T cell regulatory agent, the methods of the present disclosure may indicate a need to begin administering a CAR T cell regulatory agent to the recipient of allogeneic cells. Alternatively, the methods of the present disclosure may indicate a need to discontinue administration of a CAR T cell regulatory agent to the recipient of allogeneic cells.

In some embodiments that involve adjusting the dosage of a CAR T cell regulatory agent, the CAR T cell regulatory agent activates the CAR T cells and/or promotes survival or replication of the CAR T cells. In this case, a level of allogeneic cell DNA below a therapeutic threshold or a decrease in the level of allogeneic cell DNA in the recipient of allogeneic cells over a specific time interval is indicative of a need to increase the dose of the CAR T cell regulatory agent being administered to the recipient of allogeneic cells, or to initiate treatment with the CAR T cell regulatory agent in the case that the recipient is not already receiving treatment.

### Adjusting Monitoring of a Recipient of Allogeneic Cells

The methods of the present disclosure for determining the levels of allogeneic cell DNA in a sample from a recipient of allogeneic cells can be used to inform the need to adjust monitoring of the recipient of allogeneic cells. In general, a level of allogeneic cell DNA above or below a therapeutic threshold and/or changes in the level of allogeneic cell DNA beyond a suitable threshold value in the recipient over a specific time interval are informative with regard to determining a need to adjust monitoring of a recipient of allogeneic cells. In some embodiments, determining the status of the allogeneic cells, as described above, is informative with regard to determining a need to adjust monitoring of a recipient of allogeneic cells.

In one aspect of the present disclosure, a method of administering allogeneic cells to a recipient and adjusting monitoring of the recipient is provided. In some embodiments, the method comprises a) administering the allogeneic cells to a recipient; b) providing cell DNA from a sample obtained from the recipient; c) sequencing a panel of single nucleotide polymorphisms (SNPs) from the cell DNA, wherein the panel of SNPs is suitable for differentiating between allogeneic cell DNA and recipient-derived cell DNA; d) assaying differences in SNP allele distribution patterns in the panel as compared to expected homozygous or heterozygous distribution patterns to determine the level of allogeneic cell DNA; and e) adjusting monitoring of a recipient of allogeneic cells based on the status of the allogeneic cells. The status of the allogeneic cells may be determined as described above. For example, in some embodiments, a level of allogeneic cell DNA above a therapeutically effective threshold and/or increasing or stable over a time interval indicates a status of engraftment, expansion and/or persistence of the allogeneic cells. In some embodiments, a level of allogeneic cell DNA below a therapeutically effective threshold and/or decreasing over a time interval indicates a status of exhaustion, contraction, loss of persistence, allogeneic cell rejection, and/or graft vs. host disease. In some embodiments, a level of allogeneic cell DNA below a therapeutically effective threshold and/or decreasing over a time interval indicates disease relapse. In some alternative embodiments, instead of adjusting monitoring of a recipient of allogeneic cells, the method comprises providing information on a need to adjust monitoring of a recipient of allogeneic cells. In some alternative embodiments, instead of adjusting monitoring of a recipient of allogeneic cells, the method comprises having monitoring of a recipient of allogeneic cells adjusted.

Depending on the status of the allogeneic cells, monitoring of the recipient may be adjusted accordingly. For example, monitoring may be adjusted by increasing or decreasing the frequency of monitoring, as appropriate. Monitoring may be adjusted by altering the means of monitoring, for example, by altering the metric or assay that is used to monitor the recipient.

### Assessing Allogeneic Cell Quality

The methods of the present disclosure for determining the levels of allogeneic cell DNA in a sample from a recipient of allogeneic cells can be informative with respect to assessing the quality of allogeneic cells. In some embodiments, a method of assessing the quality of allogeneic cells is provided, the method comprising a) administering the allogeneic cells to a recipient; b) providing cell DNA from a sample obtained from the recipient; c) sequencing a panel of single nucleotide polymorphisms (SNPs) from the cell DNA, wherein the panel of SNPs is suitable for differentiating between allogeneic cell DNA and recipient-derived cell DNA; and d) assaying differences in SNP allele distribution patterns in the panel as compared to expected homozygous or heterozygous distribution patterns to determine the level of allogeneic cell DNA. In some embodiments, the level of allogeneic cell DNA is indicative of allogeneic cell quality.

Without wishing to be bound by theory, it is believed that allogeneic cells that are not viable, not capable of expanding in the recipient, or are otherwise not therapeutically effective are low quality allogeneic cells. In general, a level of allogeneic cell DNA below a therapeutically effective threshold and/or decreasing over a time interval can indicate that the allogeneic cells are poor or low quality allogeneic cells. In some cases, upon detection of poor or low quality allogeneic cells based on the level of allogeneic cell DNA, the methods of the present disclosure may further comprise assessing the quality and/or viability of the allogeneic cells through other means. In some embodiments in which the level of allogeneic cell DNA indicates poor quality allogeneic cells, the methods of the present disclosure may further comprise adjusting treatment of the recipient of the allogeneic cells. In some embodiments in which the level of allogeneic cell DNA indicates poor quality allogeneic cells, treatment may be adjusted by administering allogeneic cells that are different than those originally administered. In some embodiments in which the level of allogeneic cell DNA indicates poor quality allogeneic cells, treatment may be adjusted by administering an increased dose of the allogeneic cells. In some embodiments in which the level of allogeneic cell DNA indicates poor quality allogeneic cells, treatment may be adjusted by administering doses of the allogeneic cells more frequently. In some embodiments in which the level of allogeneic cell DNA indicates poor quality allogeneic cells, the methods of the present disclosure may further comprise improving the quality of the allogeneic cells. For example, the quality of the allogeneic cells may be improved by adjusting the manufacturing process used to produce the allogeneic cells.

### Additional Methods

In addition to the methods of determining the status of allogeneic cells and administering allogeneic cells to a recipient and adjusting treatment of the recipient, as described above, the present disclosure also provides methods of treating allogeneic cell rejection in a recipient, monitoring for relapse of a hematologic cancer in a recipient, measuring the level of chimerism in a sample, measuring a cellular kinetic parameter of allogeneic cells in a recipient, identifying allogeneic cells in a recipient, predicting recipient responsiveness to allogeneic cell administration, and identifying recipients at a higher risk for a side effect associated with allogeneic cell administration. In general, in the methods of the present disclosure, determining the status of allogeneic cells may be informative with regard to making clinical decisions surrounding the treatment of a recipient of allogeneic cells. For example, the status of allogeneic cells may be informative with regard to adjusting the treatment of a recipient of allogeneic cells, as described above.

### Treating Allogeneic Cell Rejection

In one aspect, the present disclosure provides a method of treating allogeneic cell rejection. Without wishing to be bound by theory, it is believed that allogeneic cell rejection occurs when the immune system of the recipient attacks the allogeneic cells. Accordingly, allogeneic cell rejection results in a reduction in the level of allogeneic cells, thereby resulting in a level of allogeneic cell DNA below a therapeutically effective threshold and/or decreasing over a time interval.

In some embodiments, the method of treating allogeneic cell rejection comprises determining the level of allogeneic cell DNA in a recipient using a method as disclosed herein. In some embodiments, the method comprises a) providing cell DNA from a sample obtained from a recipient of allogeneic cells; b) sequencing a panel of single nucleotide polymorphisms (SNPs) from the cell DNA, wherein the panel of SNPs is suitable for differentiating between allogeneic cell DNA and recipient-derived cell DNA; c) assaying differences in SNP allele distribution patterns in the panel as compared to expected homozygous or heterozygous distribution patterns to determine the level of allogeneic cell DNA; d) diagnosing a recipient as experiencing allogeneic cell rejection, wherein a level of allogeneic cell DNA below a therapeutically effective threshold and/or decreasing over a time interval indicates allogeneic cell rejection; and e) administering an immunosuppressive therapy or adjusting ongoing immunosuppressive therapy to the recipient diagnosed as exhibiting allogeneic cell rejection. In some embodiments, a level of allogeneic cell DNA below a therapeutically effective threshold and/or decreasing over a time interval indicates allogeneic cell rejection. In some alternative embodiments, instead of diagnosing a recipient as experiencing allogeneic cell rejection, the method comprises providing information on diagnosis of a recipient as experiencing allogeneic cell rejection. In some alternative embodiments, instead of diagnosing a recipient as experiencing allogeneic cell rejection, the method comprises having a recipient diagnosed as experiencing allogeneic cell rejection.

In some embodiments, the method of treating allogeneic cell rejection comprises administering an immunosuppressive therapy, as described above. In some embodiments, the method comprises initiating an immunosuppressive therapy, as described above. In some embodiments, the method of treating allogeneic cell rejection comprises adjusting ongoing immunosuppressive therapy. In some embodiments, the method of treating allogeneic cell rejection comprises administering an increased dose of immunosuppressive therapy. In some embodiments, the method of treating allogeneic cell rejection comprises administering a dose of allogeneic cells that are the same as those originally administered. In some embodiments, the method of treating allogeneic cell rejection comprises administering a dose of allogeneic cells that are different from those originally administered (e.g., allogeneic cells derived from a different donor). In some embodiments, the method of treating allogeneic cell rejection comprises administering to the recipient a donor lymphocyte infusion.

### Monitoring for Relapse of a Hematologic Cancer

The present disclosure also provides a method of monitoring for relapse of a hematologic cancer in a recipient. In some embodiments, the method comprises a) providing cell DNA from a sample obtained from the recipient of allogeneic cells; b) sequencing a panel of single nucleotide polymorphisms (SNPs) from the cell DNA, wherein the panel of SNPs is suitable for differentiating between allogeneic cell DNA and recipient-derived cell DNA; c) assaying differences in SNP allele distribution patterns in the panel as compared to expected homozygous or heterozygous distribution patterns to determine the level of allogeneic cell DNA; and d) monitoring for relapse based on the level of allogeneic cell DNA. In some embodiments, if there appears to be relapse of the hematologic cancer, treatment of the recipient with allogeneic cells is re-initiated, the recipient is monitored more frequently, and/or the relapse is confirmed using other measures. In some alternative embodiments, instead of monitoring for relapse based on the level of allogeneic cell DNA, the method comprises providing information on a relapse of a hematological cancer. In some alternative embodiments, instead of monitoring for relapse based on the level of allogeneic cell DNA, the method comprises having relapse of a hematological cancer monitored, or having monitoring of a hematological cancer adjusted.

In some embodiments, a level of allogeneic cell DNA below a therapeutically effective threshold and/or decreasing over a time interval indicates a relapse of a hematologic cancer. In some embodiments, treatment of the recipient with allogeneic cells is re-initiated if there is a relapse of the hematologic cancer. Hematologic cancers are cancers that begins in blood-forming tissue, such as the bone marrow, or in the cells of the immune system. In some embodiments, the hematologic cancer is a leukemia (e.g., acute lymphocytic leukemia, acute myeloid leukemia, chronic lymphocytic leukemia, chronic myeloid leukemia), a lymphoma (e.g., Hodgkin's lymphoma or non-Hodgkin's lymphoma), a myeloma (e.g., multiple myeloma), myelodysplastic syndromes (MDS), or myeloproliferative neoplasias (MPN). In some embodiments in which the level of allogeneic cell DNA indicates a relapse of a hematologic cancer, the method may further comprise re-initiating treatment of the recipient with allogeneic cells, more frequent monitoring of the recipient, and/or confirmation of the relapse using other measures (e.g., a diagnostic method). In some embodiments, a level of allogeneic cell DNA above a therapeutically effective threshold and/or increasing over a time interval indicates that a relapse of a hematologic cancer is less likely to occur. In some embodiments in which the level of allogeneic cell DNA indicates a relapse of a hematologic cancer, the method may further comprise adjustment of allogeneic cell administration (e.g, by administering an additional and/or modified dose of allogeneic cells), administration of chemotherapy, administration of targeted anti-leukemia therapy, administration of immunotherapy, and/or palliative care. In some embodiments in which the level of allogeneic cell DNA indicates a relapse of a hematologic cancer, the method may further comprise withdrawal of an immune suppression therapy, administration of a chemotherapy, a second administration of allogeneic cells (e.g., an allogeneic cell transplantation), administration of a cytokine therapy, administration of an adoptive cell therapy, and/or donor lymphocyte infusion.

### Measuring the Level of Chimerism

The present disclosure provides a method of measuring the level of chimerism in a sample. In some embodiments, the method comprises: a) providing cell DNA from a sample obtained from a recipient of allogeneic cells; b) sequencing a panel of single nucleotide polymorphisms (SNPs) from the cell DNA, wherein the panel of SNPs is suitable for differentiating between allogeneic cell DNA and recipient-derived cell DNA; c) assaying differences in SNP allele distribution patterns in the panel as compared to expected homozygous or heterozygous distribution patterns to determine the level of allogeneic cell DNA; and d) determining the level of chimerism in the sample. In some embodiments, treatment of the recipient is adjusted based on the level of chimerism in the sample. In some embodiments, the method of measuring the level of chimerism in a sample further comprises adjusting treatment of the recipient of the allogeneic cells based on the level of chimerism in the sample. In some alternative embodiments, instead of determining the level of chimerism in the sample, the method comprises providing information on the level of chimerism in the sample. In some alternative embodiments, instead of determining the level of chimerism in the sample, the method comprises having treatment of the recipient of the allogeneic cells adjusted based on the level of chimerism in the sample.

In some embodiments, the method of measuring the level of chimerism in a sample is used to assess engraftment of allogeneic cells. In some cases, the level of chimerism indicates a successful administration and/or engraftment of the allogeneic cells. In these cases, the method of measuring the level of chimerism may further comprise adjusting treatment by administering a reduced dose of the allogeneic cells, administering doses of the allogeneic cells less frequently, discontinuing administration of the allogeneic cells, or combinations thereof. In some embodiments, treatment is adjusted by reducing an immunosuppressive therapy, or discontinuing administration of an immunosuppressive therapy.

In some cases, the level of chimerism indicates that administration of allogeneic cells was not successful (e.g., that the allogeneic cells did not successfully engraft, and/or persist). In these cases, the method of measuring the level of chimerism may further comprise adjusting treatment by continuing administration of allogeneic cells that are the same as those administered, administering allogeneic cells that are different than those administered, administering an increased dose of the allogeneic cells, administering doses of the allogeneic cells more frequently, or combinations thereof. In some embodiments, treatment is adjusted by initiating an immunosuppressive therapy, or adjusting an immunosuppressive therapy.

In some embodiments, the method of measuring the level of chimerism in a sample is used to detect the relapse of a disease. In some embodiments, a level of chimerism that indicates a reduction in the level of allogeneic cell DNA and/or an increase in the level of recipient-derived DNA indicates the relapse of a disease. In some embodiments, the disease is a malignant disease. In some embodiments in which relapse of a disease is detected, the method of measuring the level of chimerism in a sample may further comprise closer surveillance of the recipient to monitor for a relapse, confirmation of relapse through an alternative method (e.g., a diagnostic method), and/or initiation of treatment for the disease.

### Measuring Cellular Kinetic Parameters

The present disclosure provides a method of measuring a cellular kinetic parameter of allogeneic cells in a recipient. In some embodiments, the method comprises a) providing cell DNA from a sample obtained from a recipient of allogeneic cells; b) sequencing a panel of single nucleotide polymorphisms (SNPs) from the cell DNA, wherein the panel of SNPs is suitable for differentiating between allogeneic cell DNA and recipient-derived cell DNA; and c) assaying differences in SNP allele distribution patterns in the panel as compared to expected homozygous or heterozygous distribution patterns to determine the level of allogeneic cell DNA; thereby measuring the cellular kinetic parameter of allogeneic cells in the recipient. In some embodiments, the method further comprises providing information on a cellular kinetic parameter of allogeneic cells in the recipient. In some embodiments, the method further comprises having treatment of the recipient of the allogeneic cells adjusted based on a cellular kinetic parameter.

In some embodiments, the cellular kinetic parameter is Cₘₐₓ. In some embodiments, the cellular kinetic parameter is the rate of contraction. In some embodiments, the cellular kinetic parameter is the rate of engraftment. In some embodiments, the cellular kinetic parameter is the AUC. In some embodiments, the cellular kinetic parameter is tₘₐₓ, i.e., the time at which Cₘₐₓ is reached. In some embodiments, the cellular kinetic parameter is a measurement of persistence. In some embodiments, the level of allogeneic cell DNA is determined according to the above method to determine the level of allogeneic cells at a point in time following administration of allogeneic cells, thereby allowing for the determination of the AUC. In some embodiments, the AUC is calculated based on the level of allogeneic cell DNA 21, 22, 23, 24, 25, 26, 27, 28, 29, 30, 31, 32, 33, 34, 35, or 36 days after administration of the allogeneic cells. In some embodiments, the AUC is calculated based on the level of allogeneic cell DNA 28 days after administration of the allogeneic cells. In some embodiments, the AUC is calculated based on the level of allogeneic cell DNA 22 days after administration of the allogeneic cells. In some embodiments, the AUC is calculated based on the level of allogeneic cell DNA 32 days after administration of the allogeneic cells. In some embodiments, the AUC is calculated based on the level of allogeneic cell DNA four weeks after administration of the allogeneic cells.

The cellular kinetic parameter (e.g., Cₘₐₓ, AUC, rate of engraftment, rate of contraction, tₘₐₓ, or a measurement of persistence) may be informative with regard to the status of the allogeneic cells. Further, the cellular kinetic parameter may be informative with regard to making a clinical decision such as whether to adjust treatment of a recipient of allogeneic cells. In some embodiments, a cellular kinetic parameter above a therapeutically effective threshold and/or increasing or stable over a time interval indicates a status of engraftment, expansion and/or persistence of the allogeneic cells. In some embodiments, a cellular kinetic parameter below a therapeutically effective threshold and/or decreasing over a time interval indicates a status of exhaustion, contraction, loss of persistence, allogeneic cell rejection, and/or graft vs. host disease. In some embodiments, the method of measuring a cellular kinetic parameter further comprises adjusting treatment of the recipient of the allogeneic cells based on the measurement of the cellular kinetic parameter. Depending on the cellular kinetic parameter, in some embodiments, a cellular kinetic parameter is informative with regard to an adjustment to treatment of the recipient, e.g., with respect to making a clinical decision. For example, in some embodiments in which the cellular kinetic parameter is above a therapeutically effective threshold and/or increasing or stable over a time interval, treatment is adjusted by administering a reduced dose of the allogeneic cells, administering doses of the allogeneic cells less frequently, discontinuing administration of the allogeneic cells, or combinations thereof. In some embodiments in which the cellular kinetic parameter is above a therapeutically effective threshold and/or increasing or stable over a time interval, treatment is adjusted by reducing an immunosuppressive therapy, or discontinuing administration of an immunosuppressive therapy. In some embodiments in which the cellular kinetic parameter is below a therapeutically effective threshold and/or decreasing over a time interval, treatment is adjusted by continuing administration of allogeneic cells that are the same as those originally administered, administering allogeneic cells that are different than those originally administered, administering an increased dose of the allogeneic cells, administering doses of the allogeneic cells more frequently, or combinations thereof. In some embodiments in which cellular kinetic parameter is below a therapeutically effective threshold and/or decreasing over a time interval, treatment is adjusted by initiating an immunosuppressive therapy, or adjusting an immunosuppressive therapy. In some embodiments in which Cₘₐₓ is below a therapeutically effective threshold, an additional dose of the same allogeneic cells is administered. In some embodiments in which Cₘₐₓ is below a therapeutically effective threshold, a dose of allogeneic cells from a different donor is administered. In some embodiments in which a measurement of persistence is below a therapeutically effective threshold, an additional dose of the allogeneic cells from the same or different donor is administered.

### Identifying Allogeneic Cells

The present disclosure provides a method of identifying allogeneic cells in a recipient. In some embodiments, the method of identifying allogeneic cells in a recipient comprises a) providing cell DNA from a sample obtained from a recipient of allogeneic cells; b) sequencing a panel of single nucleotide polymorphisms (SNPs) from the cell DNA, wherein the panel of SNPs is suitable for differentiating between allogeneic cell DNA and recipient-derived cell DNA; and c) assaying differences in SNP allele distribution patterns in the panel as compared to expected homozygous or heterozygous distribution patterns to identify the allogeneic cell DNA and ensure that the correct allogeneic cells were administered.

In some embodiments, the method of identifying allogeneic cells further comprises adjusting treatment of the recipient of the allogeneic cells based on the identification of the allogeneic cells. In some embodiments, the method comprises providing information on the identification of the allogeneic cells. In some embodiments, the method comprises having treatment of the recipient of the allogeneic cells adjusted based on the identification of the allogeneic cells.

### Predicting Recipient Responsiveness to Allogeneic Cell Administration

The present disclosure provides a method of predicting recipient responsiveness to allogeneic cell administration. In some embodiments, the method comprises a) providing cell DNA from a sample obtained from a recipient of allogeneic cells; b) sequencing a panel of single nucleotide polymorphisms (SNPs) from the cell DNA, wherein the panel of SNPs is suitable for differentiating between allogeneic cell DNA and recipient-derived cell DNA; and c) assaying differences in SNP allele distribution patterns in the panel as compared to expected homozygous or heterozygous distribution patterns to determine the level of allogeneic cell DNA. In some embodiments, a level of allogeneic cell DNA above a therapeutically effective threshold and/or increasing or stable over a time interval indicates that it is more likely that the recipient will respond to the allogeneic cells, and a level of allogeneic cell DNA below a therapeutically effective threshold and/or decreasing over a time interval indicates that it is less likely that the recipient will respond to the allogeneic cells. In some embodiments, the method comprises providing information on the recipient responsiveness to allogeneic cell administration. In some embodiments, the method comprises having treatment of the recipient of the allogeneic cells adjusted based on the recipient responsiveness to allogeneic cell administration.

A recipient that is responsive to allogeneic cell administration is one in which the intended therapeutic effect of the allogeneic cells is achieved. Without wishing to be bound by theory, it is believed that the level of allogeneic cells in a recipient correlates to recipient responsiveness. In other words, it is believed that a recipient with a higher level of allogeneic cells is more likely to respond to the allogeneic cells. Accordingly, in some embodiments, a level of allogeneic cell DNA above a therapeutically effective threshold and/or increasing or stable over a time interval indicates that it is more likely that the recipient will respond to the allogeneic cells. In some embodiments, a level of allogeneic cell DNA below a therapeutically effective threshold and/or decreasing over a time interval indicates that it is less likely that the recipient will respond to the allogeneic cells.

In some embodiments, the method of predicting recipient responsiveness to allogeneic cell administration further comprises adjusting treatment of the recipient of the allogeneic cells based on the predicted recipient responsiveness to allogeneic cell administration. Adjusting the treatment may occur through any of the methods described herein, as appropriate. For example, in some embodiments in which the recipient is predicted to be less likely to respond to the allogeneic cells, the administration of allogeneic cells may be adjusted. In some embodiments in which the recipient is predicted to be less likely to respond, administration of allogeneic cells is adjusted by continuing administration of allogeneic cells that are the same as those originally administered, administering allogeneic cells that are different than those originally administered, administering an increased dose of the allogeneic cells, administering doses of the allogeneic cells more frequently, or combinations thereof. In some embodiments in which the recipient is predicted to be less likely to respond, treatment is adjusted by initiating an immunosuppressive therapy, or adjusting an immunosuppressive therapy. In some embodiments in which the recipient is predicted to be less likely to respond, treatment is adjusted by administering a different therapy, such as a chemotherapy, a targeted anti-leukemia therapy, immunotherapy, or palliative care. In some embodiments in which the recipient is predicted to be more likely to respond to the allogeneic cells, treatment is adjusted by administering a reduced dose of the allogeneic cells, administering doses of the allogeneic cells less frequently, discontinuing administration of the allogeneic cells, or combinations thereof. In some embodiments in which the recipient is predicted to be more likely to respond to the allogeneic cells, treatment is adjusted by reducing an immunosuppressive therapy, or discontinuing administration of an immunosuppressive therapy.

Identifying Recipients at a Higher Risk for a Side Effect Associated with Allogeneic Cell Administration

The present disclosure provides a method of identifying recipients at a higher risk for a side effect associated with allogeneic cell administration. In some embodiments, the side effect associated with allogeneic cell administration is graft vs. host disease, chronic graft vs. host disease, acute graft vs. host disease, host vs. graft disease, cytokine release syndrome, fatigue, fever, headache, chill, flushing, nausea, bone pain, avascular necrosis, increased risk of infection, pneumonitis, hepatic veno-occlusive disease, cancer relapse, secondary cancer, cataracts, or changes in the thyroid or pituitary gland. In some embodiments, the side effect is an adverse effect or a serious adverse effect. In some embodiments, the side effect is a neurologic side effect such as encephalopathy (e.g., brain disease, injury, malfunction), confusion, aphasia (i.e., difficulty understand or speaking), drowsiness, agitation, seizures, loss of balance, or altered consciousness. In some embodiments, the side effect is a low white blood cell count (i.e., neutropenia). In some embodiments, the side effect is a low red blood cell count (i.e., anemia). In some embodiments, the method of identifying recipients at a higher risk for a side effect associated with allogeneic cell administration comprising: a) providing cell DNA from a sample obtained from a recipient of allogeneic cells; b) sequencing a panel of single nucleotide polymorphisms (SNPs) from the cell DNA, wherein the panel of SNPs is suitable for differentiating between allogeneic cell DNA and recipient-derived cell DNA; and c) assaying differences in SNP allele distribution patterns in the panel as compared to expected homozygous or heterozygous distribution patterns to determine the level of allogeneic cell DNA.

In general, the level of allogeneic cell DNA can be above or below a safety threshold, and thereby be informative with regard to whether a recipient is at risk for a side effect associated with allogeneic cell administration. In some embodiments, a level of allogeneic cell DNA above a safety threshold indicates that the recipient is at a higher risk of a side effect associated with allogeneic cell administration, thereby identifying recipients at a higher risk for a side effect associated with allogeneic cell administration. In some embodiments, a level of allogeneic cell DNA below safety threshold indicates that the recipient is at a lower risk of a side effect associated with allogeneic cell administration, thereby identifying recipients at a lower risk for a side effect associated with allogeneic cell administration.

In some embodiments, the method of identifying recipients at a higher risk for a side effect associated with allogeneic cell administration further comprises adjusting treatment of the recipient of the allogeneic cells based on the risk for a side effect associated with allogeneic cell administration. In some embodiments, the method of identifying recipients at a higher risk for a side effect associated with allogeneic cell administration further comprises adjusting the treatment of the recipient, as appropriate. For example, in some embodiments in which the recipient is at a lower risk of a side effect associated with allogeneic cell administration, the administration of allogeneic cells may be adjusted. In some embodiments in which the recipient is at a lower risk of a side effect associated with allogeneic cell administration, administration of allogeneic cells is adjusted by continuing administration of allogeneic cells that are the same as those originally administered, administering an increased dose of the allogeneic cells, administering doses of the allogeneic cells more frequently, or combinations thereof.

In some embodiments in which the recipient is at a higher risk of a side effect associated with allogeneic cell administration, the administration of allogeneic cells may be reduced or ceased. In some embodiments in which the recipient is at a higher risk of a side effect associated with allogeneic cell administration, treatment of the recipient of the allogeneic cells is adjusted by administering a reduced dose of the allogeneic cells, administering doses of the allogeneic cells less frequently, and/or discontinuing administration of the allogeneic cells. In some embodiments in which the recipient is at a higher risk of a side effect associated with allogeneic cell administration, treatment is adjusted by initiating an immunosuppressive therapy, or adjusting an immunosuppressive therapy. In some embodiments in which the recipient is at a higher risk of a side effect associated with allogeneic cell administration, the recipient is monitored for the development of a side effect. In some embodiments in which the recipient is at a higher risk of a side effect associated with allogeneic cell administration, the recipient is prophylactically treated. In some embodiments in which the recipient is at a higher risk of a side effect associated with allogeneic cell administration, the method of identifying recipients at a higher risk for a side effect associated with allogeneic cell administration further comprises performing further methods to detect a side effect.

In some embodiments, the method of identifying recipients at a higher risk for a side effect associated with allogeneic cell administration comprises providing information on the identification of a recipient at a higher risk for a side effect associated with allogeneic cell administration. In some embodiments, the method comprises having treatment of the recipient of the allogeneic cells adjusted based on the identification of a recipient at a higher risk for a side effect associated with allogeneic cell administration.

### Assessing Therapeutic Effectiveness

The present disclosure provides a method of assessing therapeutic effectiveness of allogeneic cells in a recipient. In some embodiments, the method comprises a) providing cell DNA from a sample obtained from the recipient of allogeneic cells; b) sequencing a panel of single nucleotide polymorphisms (SNPs) from the cell DNA, wherein the panel of SNPs is suitable for differentiating between allogeneic cell DNA and recipient-derived cell DNA; c) assaying differences in SNP allele distribution patterns in the panel as compared to expected homozygous or heterozygous distribution patterns to determine the level of allogeneic cell DNA; and d) assessing therapeutic effectiveness based on the level of allogeneic cell DNA. In some embodiments, the method further comprises providing information on the therapeutic effectiveness of the allogeneic cells in a recipient. In some embodiments, the method further comprises having treatment of the recipient of the allogeneic cells adjusted based on the therapeutic effectiveness of the allogeneic cells in a recipient.

In general, as described above, a level of allogeneic cell DNA above a therapeutically effective threshold and/or increasing or stable over a time interval indicates therapeutic effectiveness, and a level of allogeneic cell DNA below a therapeutically effective threshold and/or decreasing over a time interval indicates a lack of therapeutic effectiveness. In some embodiments, the method of assessing therapeutic effectiveness of allogeneic cells in a recipient is performed in the context of a clinical trial. In some embodiments, the clinical trial is an allogeneic cell therapy clinical trial, such as a CAR T cell therapy clinical trial or an HCT clinical trial.

### EXAMPLES

### Example 1: Monitoring Chimerism by Detection of Allogeneic Cell DNA

The following example describes the detection of chimerism in a sample.

### Methods

To generate samples representing chimeric cell mixtures, genomic DNAs extracted from two different (i.e., genetically distinguishable) individuals were mixed at pre-defined target ratios ranging from 0-100%. Specifically, genomic DNAs from the two individuals (NA16689 and NA17070) were combined and serially diluted with NA17070 genomic DNA to represent allogeneic cell to recipient cell ratios of 0, 0.01, 0.03, 0.1, 0.3, 0.6, 1, 3, 5, 10, 15, 20, 25, 20, 35, 40, 45, 50 and 100%. Generated mixtures were each analyzed in 5 replicates. The mixtures were PCR-amplified using primers targeting 405 SNPs and labeled with sample-specific molecular barcode tags to generate sequencing libraries. The sequencing libraries were then pooled and sequenced using next-generation sequencing-by-synthesis. Sequencing data was analyzed using an algorithm that calculates the percentage of the genome mixture.

### Results

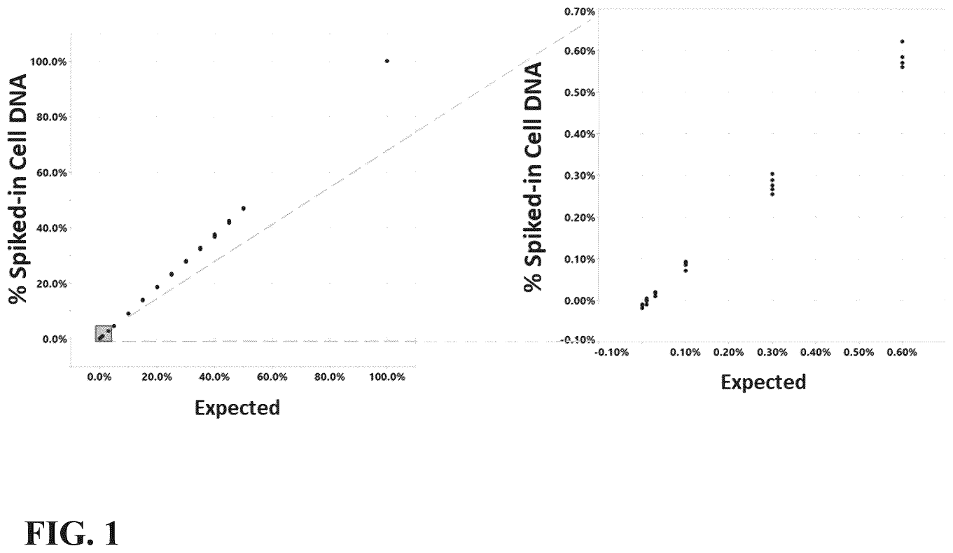

As shown in FIG. 1, the percentage of allogeneic cell DNA was measured accurately in a mixture containing genomic DNAs extracted from two different human cell lines.

### Example 2: Monitoring Allogeneic Cell Therapy by Detection of Cell DNA

The following example describes a method intended for cell therapy patients, such as recipients of TCR or CAR T cells, NK cells, NKT cells, mesenchymal stem cells, or any other cell therapy where majority of the blood cells in the patient are expected to consist of patient's cells. The amount of cell therapy product is measured relative to patient cells, based on analysis of hundreds of SNPs found across human genome (see, e.g., FIG. 2).

The laboratory workflow for performing the method is as follows. DNA is extracted from cells, for example, from whole blood or subpopulations of cells, e.g., CD3+ cells. The SNPs are amplified by multiplex PCR. The amplified SNP loci are then sequenced, for example, by next-generation sequencing such as Illumina sequencing. The fraction of differing nucleotides is calculated using an algorithm, such as that described herein.

Cell therapy product kinetics, engraftment post-infusion, disappearance of cells from peripheral blood (presumably due to redistribution to tissues), expansion, peak expansion, time to peak expansion, contraction, and/or persistence are measured. The measurements enable clinical decisions as described herein, such as optimizing the level and frequency of doses, switching to a different cell donor, identifying a risk of adverse effects, and/or identifying a risk of relapse (for example, through persistence monitoring).

### Example 3: Monitoring Cell Transplantation by Detection of Cell DNA

The following example describes a method intended for patients that have received bone marrow, cord blood, or hematopoietic stem cell transplantations, where majority of the blood cells in the patient are expected to consist of donor cells. The amount of cell product is measured relative to patient cells, based on analysis of hundreds of SNPs found across human genome.

The laboratory workflow for performing the method is as follows. DNA is extracted from cells, for example, whole blood or subpopulations of cells, e.g., CD3+ cells. The SNPs are amplified by multiplex PCR. The amplified SNP loci are then sequenced, for example, by next-generation sequencing such as Illumina sequencing. The fraction of differing nucleotides is calculated using an algorithm, such as that described herein.

The level of chimerism informs clinical decisions, as described herein. For example, the level of chimerism is measured to assess cell engraftment post-infusion and/or detect disease relapse.

### Example 4: Monitoring Chimerism in Blood Samples

The following example describes the detection of chimerism in blood samples.

### Methods

Blood was collected in K2EDTA tubes from ten healthy volunteers. Five blood panels termed AB, CD, EF, GH and IJ were each generated by combining pre-specified volumes of blood from two volunteers at various proportions (see Table 1). The AB panel was generated by combining blood from related volunteers (parent and child pair). The other panels were generated by mixing blood from unrelated subjects.

Blood samples were combined at the proportions shown in Table 1, below.

TABLE 1
Proportions used in blood panels

| Proportion of major contributor | Proportion of minor contributor |
|---|---|
| 99.99 | 0.01 |
| 99.97 | 0.03 |
| 99.95 | 0.05 |
| 99.9 | 0.1 |
| 99.7 | 0.3 |
| 99.5 | 0.5 |
| 99 | 1 |
| 97 | 3 |
| 95 | 5 |
| 90 | 10 |

Genomic cell DNA was extracted in triplicate from the individual panels and was analyzed at 8 and 100 ng input. DNA was PCR-amplified using primers targeting 405 SNPs and labeled with sample-specific molecular barcode tags to generate sequencing libraries. The sequencing libraries were then pooled and sequenced using next-generation sequencing-by-synthesis. Sequencing data was analyzed using an algorithm that calculates the percentage of the genome mixture.

### Results

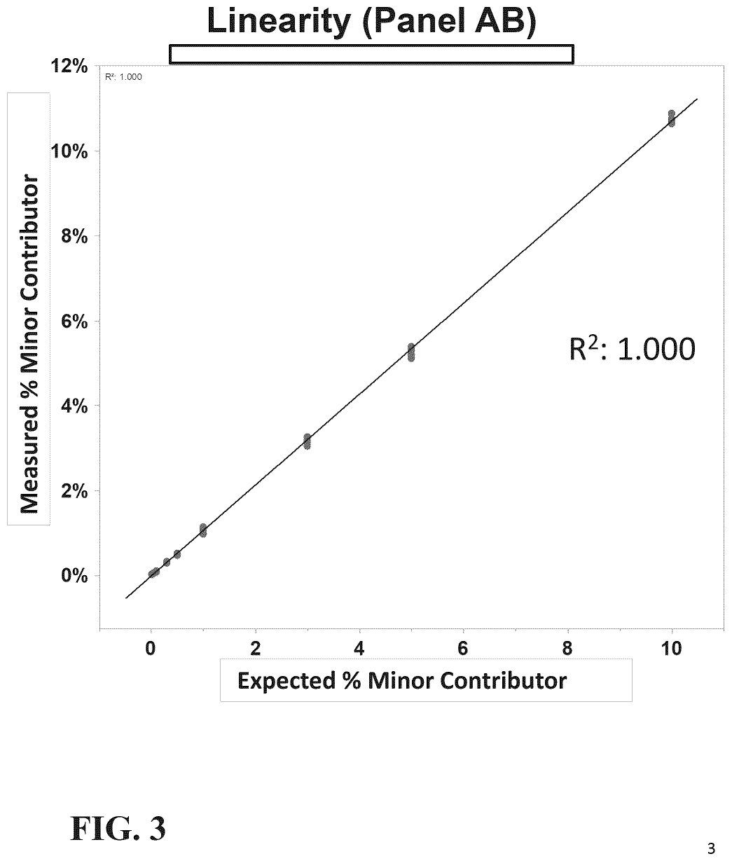

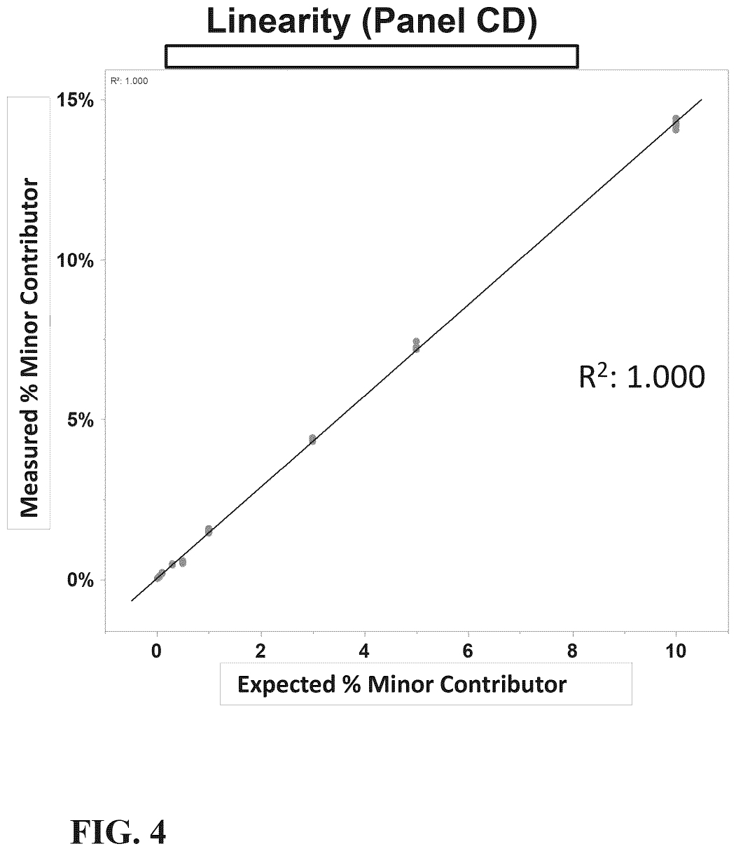

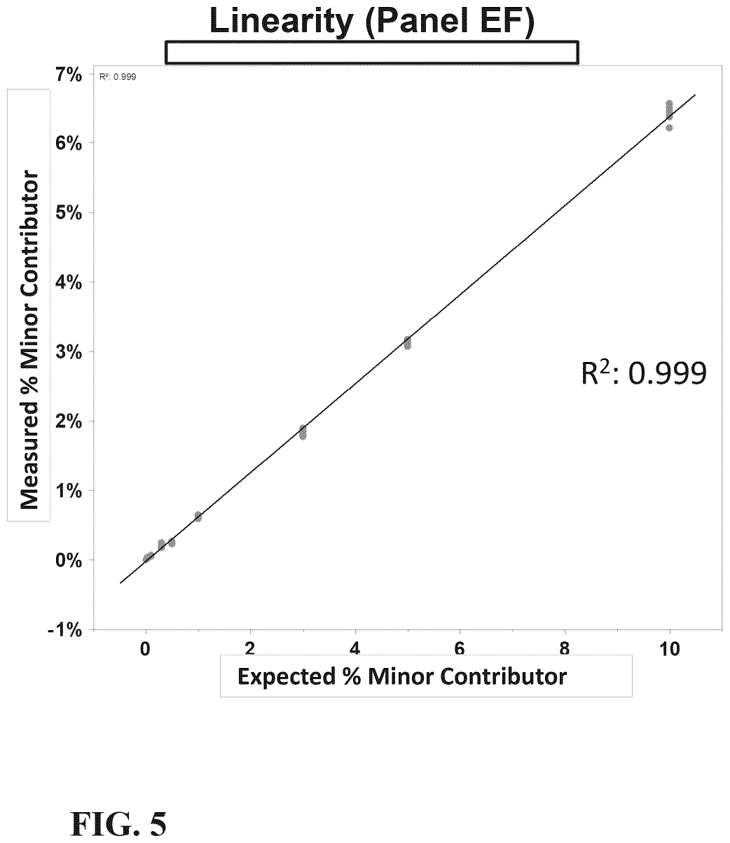

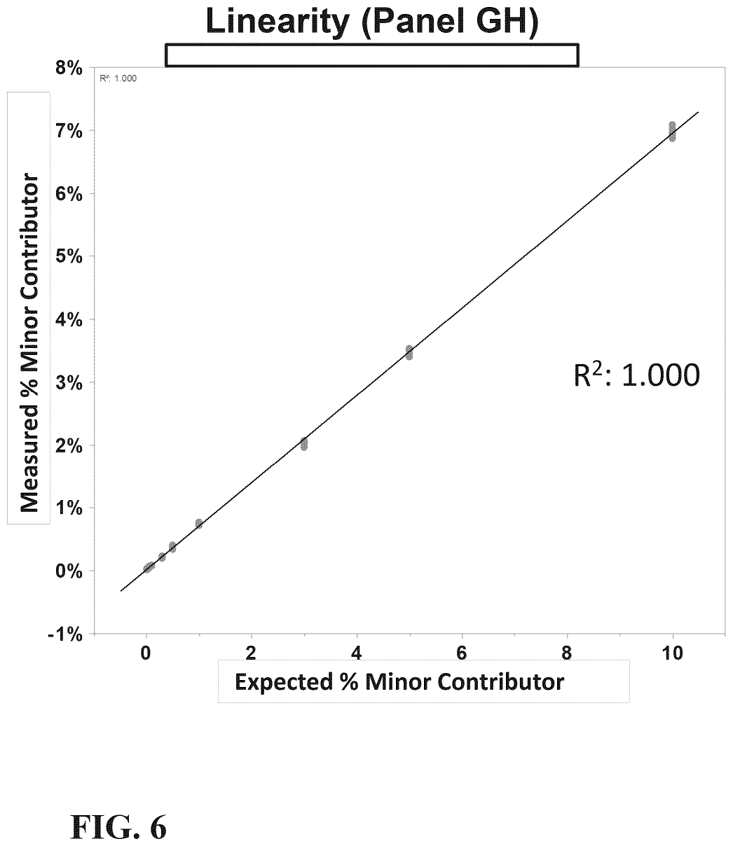

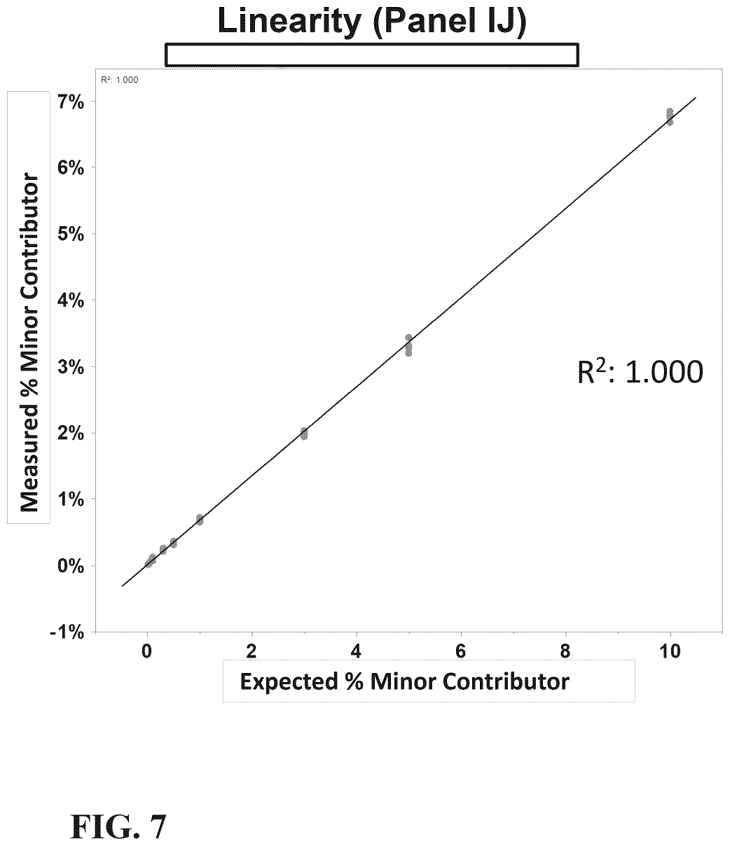

As shown in FIGS. 3-7, the percentage of allogeneic cell DNA was measured accurately in a linear fashion for all five of the blood panels. Slight differences between expected and measured are in accordance with variable number of cells present in blood of different individuals. The R² value for panel AB was 1.000, the R² value for panel CD was 1.000, the R² value for panel EF was 0.999, the R² value for panel GH was 1.000, and the R² value for panel IJ was 1.000. Accordingly, the method accurately measured levels of chimerism in blood samples with a high level of linearity, in blood mixtures from both related (panel AB) and unrelated (panels CD, EF, GH and IJ) individuals.

### Example 5: Monitoring Chimerism of Specific Cell Types in Blood Samples

The following example describes the detection of chimerism in blood samples, in particular in CD3+, CD15+, and CD33+ cell subtypes. In addition, the experiments described in this example demonstrate that equivalent chimerism results are obtained when DNA used from either buccal swab or whole blood was used as a reference.

### Methods

Blood was collected in K2EDTA tubes from 20 healthy volunteers. Equal volumes of blood from two unrelated individuals were mixed to generate 10 mixed samples (A1B1, C1D1, E1F1, MN, OP, QR, ST, UV, WX and YZ).

Cell enrichment for CD3+, CD15+ and CD33+ cell subtypes was performed in triplicates from each mixture using cell subtype-specific antibodies and magnetic beads. Genomic cell DNA was extracted from enriched cell subtypes and analyzed at 8 ng (“low input”) and 100-150 ng (“high input”), for a total of 6 replicates per each cell subtype mixture. The mixtures were PCR-amplified using primers targeting 405 SNPs and labeled with sample-specific molecular barcode tags to generate sequencing libraries. The sequencing libraries were then pooled and sequenced using next-generation sequencing-by-synthesis.

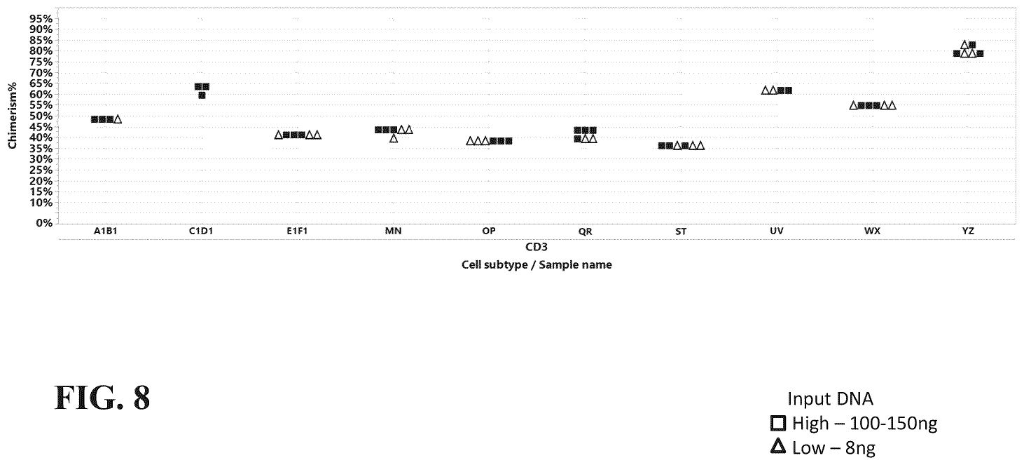

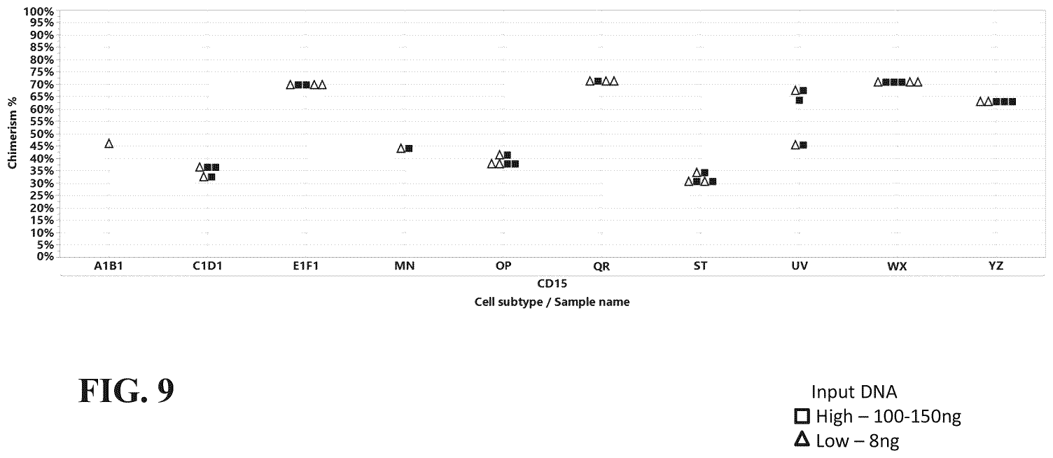

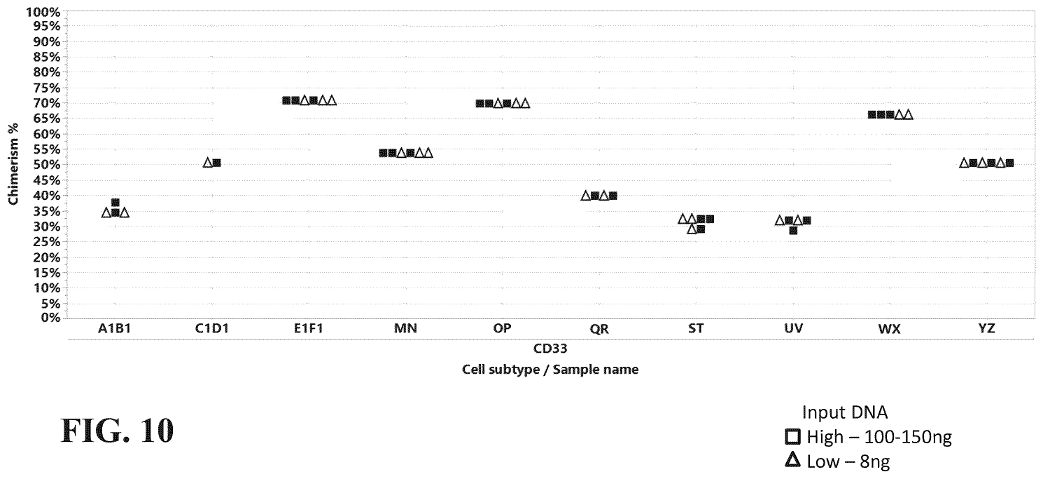

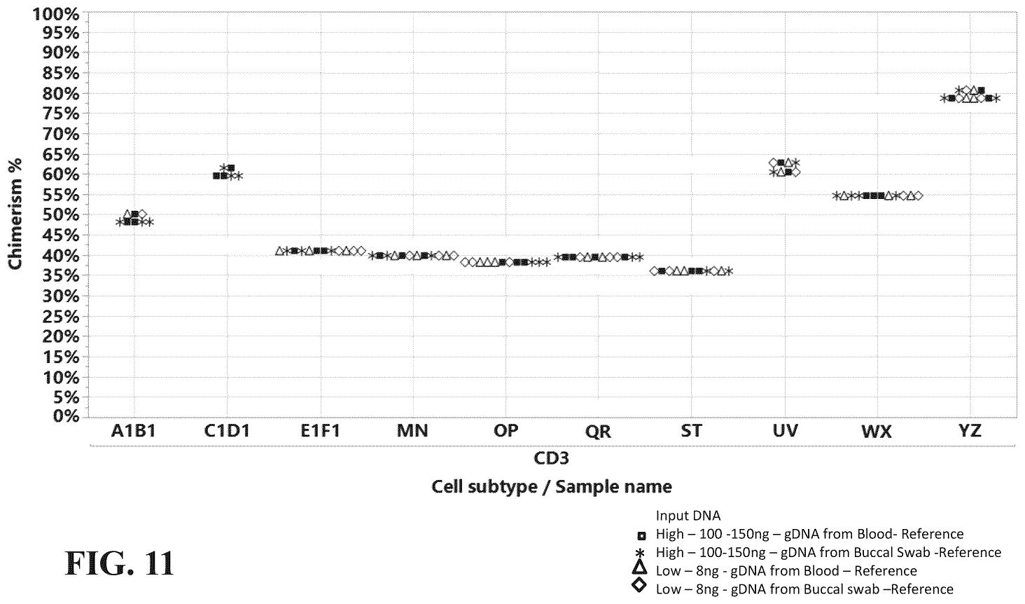

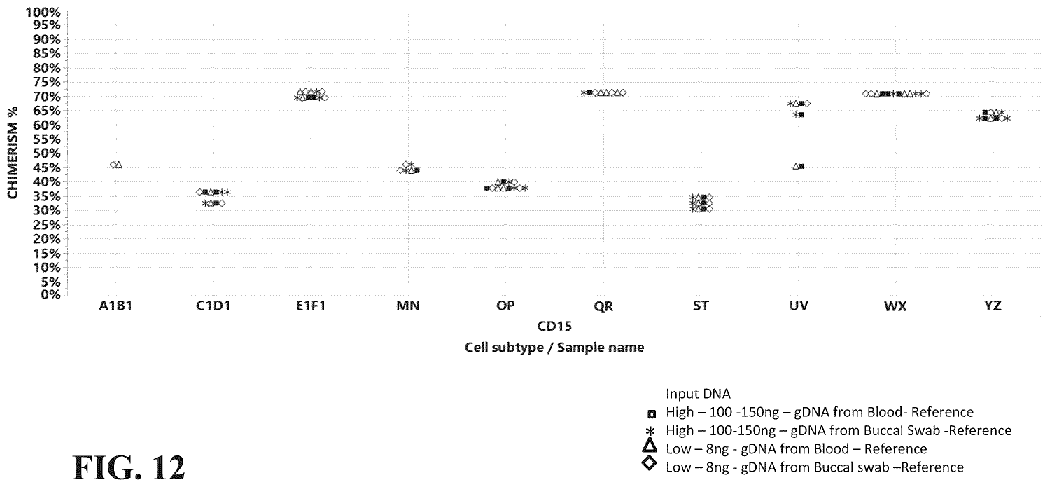

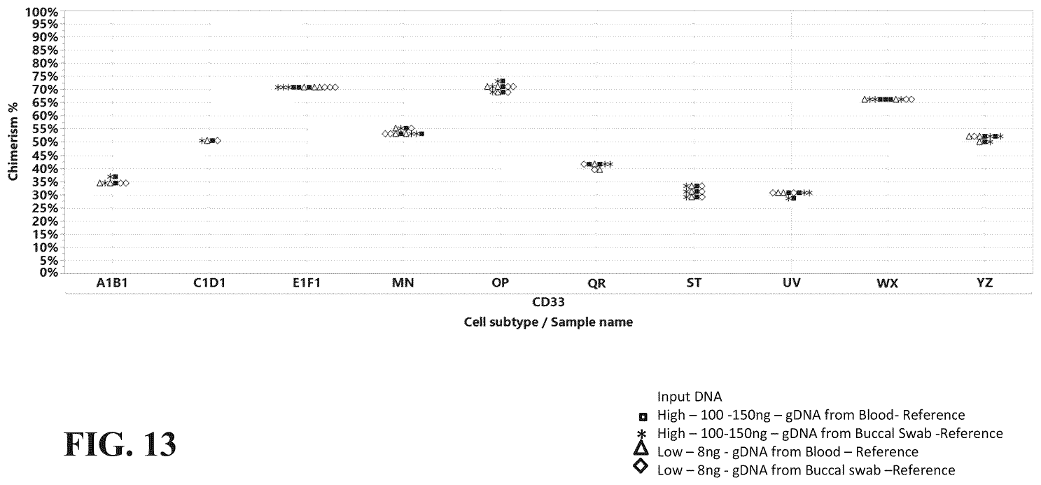

The level of chimerism in CD3+, CD15+, or CD33+ cell subtypes was determined from sequencing data using an algorithm that calculates the percentage of the genome mixtures (FIGS. 8-10). In addition, the level of chimerism in CD3+, CD15+, or CD33+ cell subtypes was determined using DNA reference samples (representing patient's genotype) derived from either whole blood or buccal swabs (FIGS. 11-13).

### Results

As shown in FIGS. 8-10, the percentage of chimerism in CD3+, CD15+, or CD33+ cell subtypes varied between different blood mixtures and different cell subtypes within a blood mixture, as expected and demonstrated biological variability of blood composition in different individuals. However, the results were highly consistent between the replicates and between the high (100-150 ng) and low (8 ng) DNA amount used. Therefore, the method was highly reproducible across the entire process of cell subtype enrichment, DNA extraction, amplification and sequencing analysis. Furthermore, the amount of DNA input did not affect the accuracy of the measurement of the percentage of chimerism.

As shown in FIGS. 11-13, the percent chimerism results in CD3+, CD15+, or CD33+ cell subtypes were not affected by the origin of the DNA reference sample used, as DNA derived from either whole blood or buccal swab yielded similar percent chimerism results.

### Example 6: Monitoring Chimerism of Cell DNA from Three Individuals

The following example describes the detection of chimerism in samples with DNA from three individuals, representing, for example, a situation in which one individual is administered two genetically distinguishable types of allogeneic cells, either in tandem or in succession.

### Methods

To generate samples representing chimeric cell mixtures from three individuals, genomic cell DNAs from three different (i.e., genetically distinguishable) individuals were mixed at pre-defined target ratios ranging from 0.1-60% to generate four different panels; C, D, G and H; each containing 29, 29, 11 and 16 different mixtures, respectively.

Panels C, D, G, and H were generated using DNA obtained from the Coriell Institute for Medical Research, New Jersey, US. Panels C and D combined three genome mixtures from unrelated individuals. Panels G and H combined three genome mixtures from two related (parent and child) and an unrelated individual. gDNA samples were combined at the proportions shown in Tables 2-5, below. Each of Tables 2-5 shows the name of the mixtures at the different proportions, the percentage of target DNA from each of the three individuals, and the sample size (N) for the 8 ng and 100 ng samples.

TABLE 2
Proportions used in Panel C (three genomes, unrelated)

| | Target | Target | Target | N, | N, |
|---|---|---|---|---|---|
| Mixture | DNA1% | DNA2% | DNA3% | 8 ng | 100 ng |
| C1 | 60.00% | 20.00% | 20.00% | 6 | 3 |
| C2 | 30.00% | 30.00% | 40.00% | 6 | 3 |
| C3 | 3.00% | 30.00% | 67.00% | 5 | 3 |
| C4 | 1.00% | 30.00% | 69.00% | 6 | 3 |
| C5 | 0.30% | 30.00% | 69.70% | 6 | 3 |
| C6 | 0.10% | 30.00% | 69.90% | 6 | 3 |
| C7 | 30.00% | 35.00% | 35.00% | 6 | 3 |
| C8 | 3.00% | 49.00% | 48.00% | 6 | 3 |
| C9 | 1.00% | 49.00% | 50.00% | 6 | 3 |
| C10 | 0.30% | 49.00% | 50.70% | 6 | 3 |
| C11 | 0.10% | 49.00% | 50.90% | 5 | 3 |
| C12 | 60.00% | 0.10% | 39.90% | 6 | 3 |
| C13 | 30.00% | 0.10% | 69.90% | 5 | 3 |
| C14 | 3.00% | 0.10% | 96.90% | 6 | 3 |
| C15 | 1.00% | 0.10% | 98.90% | 6 | 2 |
| C16 | 0.30% | 0.10% | 99.60% | 6 | 3 |
| C17 | 0.10% | 0.10% | 99.80% | 6 | 3 |
| C18 | 60.00% | 0.30% | 39.70% | 6 | 3 |
| C19 | 30.00% | 0.30% | 69.70% | 6 | 3 |
| C20 | 3.00% | 0.30% | 96.70% | 5 | 3 |
| C21 | 1.00% | 0.30% | 98.70% | 6 | 3 |
| C22 | 0.30% | 0.30% | 99.40% | 6 | 3 |
| C23 | 0.10% | 0.30% | 99.60% | 6 | 3 |
| C24 | 60.00% | 1.00% | 39.00% | 6 | 3 |
| C25 | 30.00% | 1.00% | 69.00% | 6 | 3 |
| C26 | 3.00% | 1.00% | 96.00% | 6 | 3 |
| C27 | 1.00% | 1.00% | 98.00% | 6 | 3 |
| C28 | 0.30% | 1.00% | 98.70% | 6 | 3 |
| C29 | 0.10% | 1.00% | 98.90% | 6 | 3 |

TABLE 3
Proportions used in Panel D (three genomes, unrelated)

| | Target | Target | Target | N, | N, |
|---|---|---|---|---|---|
| Mixture | DNA1% | DNA2% | DNA3% | 8 ng | 100 ng |
| D1 | 60.00% | 20.00% | 20.00% | 6 | 6 |
| D2 | 30.00% | 30.00% | 40.00% | 6 | 6 |
| D3 | 3.00% | 30.00% | 67.00% | 6 | 5 |
| D4 | 0.60% | 30.00% | 69.40% | 6 | 6 |
| D5 | 0.30% | 30.00% | 69.70% | 6 | 6 |
| D6 | 0.10% | 30.00% | 69.90% | 6 | 6 |
| D7 | 30.00% | 35.00% | 35.00% | 6 | 3 |
| D8 | 3.00% | 49.00% | 48.00% | 6 | 3 |
| D9 | 1.00% | 49.00% | 50.00% | 6 | 3 |
| D10 | 0.30% | 49.00% | 50.70% | 6 | 3 |
| D11 | 0.10% | 49.00% | 50.90% | 6 | 3 |
| D12 | 60.00% | 0.10% | 39.90% | 6 | 3 |
| D13 | 30.00% | 0.10% | 69.90% | 6 | 2 |
| D14 | 3.00% | 0.10% | 96.90% | 6 | 3 |
| D15 | 1.00% | 0.10% | 98.90% | 6 | 3 |
| D16 | 0.30% | 0.10% | 99.60% | 6 | 2 |
| D17 | 0.10% | 0.10% | 99.80% | 6 | 3 |
| D18 | 60.00% | 0.30% | 39.70% | 5 | 3 |
| D19 | 30.00% | 0.30% | 69.70% | 6 | 3 |
| D20 | 3.00% | 0.30% | 96.70% | 3 | 5 |
| D21 | 1.00% | 0.30% | 98.70% | 6 | 3 |
| D22 | 0.30% | 0.30% | 99.40% | 6 | 3 |
| D23 | 0.10% | 0.30% | 99.60% | 6 | 3 |
| D24 | 60.00% | 1.00% | 39.00% | 6 | 6 |
| D25 | 30.00% | 1.00% | 69.00% | 6 | 6 |
| D26 | 3.00% | 1.00% | 96.00% | 11 | 11 |
| D27 | 0.60% | 1.00% | 98.40% | 6 | 6 |
| D28 | 0.30% | 1.00% | 98.70% | 11 | 11 |
| D29 | 0.10% | 1.00% | 98.90% | 6 | 6 |

TABLE 4
Proportions used in Panel G (three genomes,
two related (parent/child) and one unrelated)

| | Target | Target | Target | N, | N, |
|---|---|---|---|---|---|
| Mixture | DNA1% | DNA2% | DNA3% | 8 ng | 100 ng |
| G1 | 60.0% | 20.0% | 20.0% | 6 | 3 |
| G2 | 30.0% | 30.0% | 40.0% | 6 | 3 |
| G3 | 3.0% | 30.0% | 67.0% | 6 | 3 |
| G4 | 1.0% | 30.0% | 69.0% | 6 | 3 |
| G5 | 0.3% | 30.0% | 69.7% | 6 | 3 |
| G6 | 0.1% | 30.0% | 69.9% | 6 | 3 |
| G7 | 30.0% | 35.0% | 35.0% | 6 | 3 |
| G8 | 3.0% | 49.0% | 48.0% | 6 | 3 |
| G9 | 1.0% | 49.0% | 50.0% | 6 | 3 |
| G10 | 0.3% | 49.0% | 50.7% | 6 | 3 |
| G11 | 0.1% | 49.0% | 50.9% | 6 | 3 |

TABLE 5
Proportions used in Panel H (three genomes,
two related (parent/child) and one unrelated)

| | Target | Target | Target | N, | N, |
|---|---|---|---|---|---|
| Mixture | DNA1% | DNA2% | DNA3% | 8 ng | 100 ng |
| H1 | 60.0% | 20.0% | 20.0% | 6 | 3 |
| H2 | 30.0% | 30.0% | 40.0% | 6 | 3 |
| H3 | 3.0% | 30.0% | 67.0% | 6 | 3 |
| H4 | 1.0% | 30.0% | 69.0% | 6 | 3 |
| H5 | 0.3% | 30.0% | 69.7% | 6 | 3 |
| H6 | 0.1% | 30.0% | 69.9% | 5 | 3 |
| H7 | 30.0% | 35.0% | 35.0% | 6 | 3 |
| H8 | 3.0% | 49.0% | 48.0% | 6 | 3 |
| H9 | 1.0% | 49.0% | 50.0% | 6 | 2 |
| H10 | 0.3% | 49.0% | 50.7% | 6 | 3 |
| H11 | 0.1% | 49.0% | 50.9% | 6 | 3 |
| H12 | 60.0% | 1.0% | 39.0% | 6 | 3 |
| H13 | 30.0% | 1.0% | 69.0% | 6 | 3 |
| H14 | 3.0% | 1.0% | 96.0% | 4 | 2 |
| H15 | 1.0% | 1.0% | 98.0% | 4 | 3 |
| H16 | 0.3% | 1.0% | 98.7% | 6 | 3 |

### Results

Using 8 ng input or 100 ng input, percentages of each of the three genomic DNA contributors (1, 2, and 3) within the various panels were evaluated by droplet digital PCR (“Expected DNA %) and compared to the assayed level of allogeneic cell DNA (“Measured DNA %”).

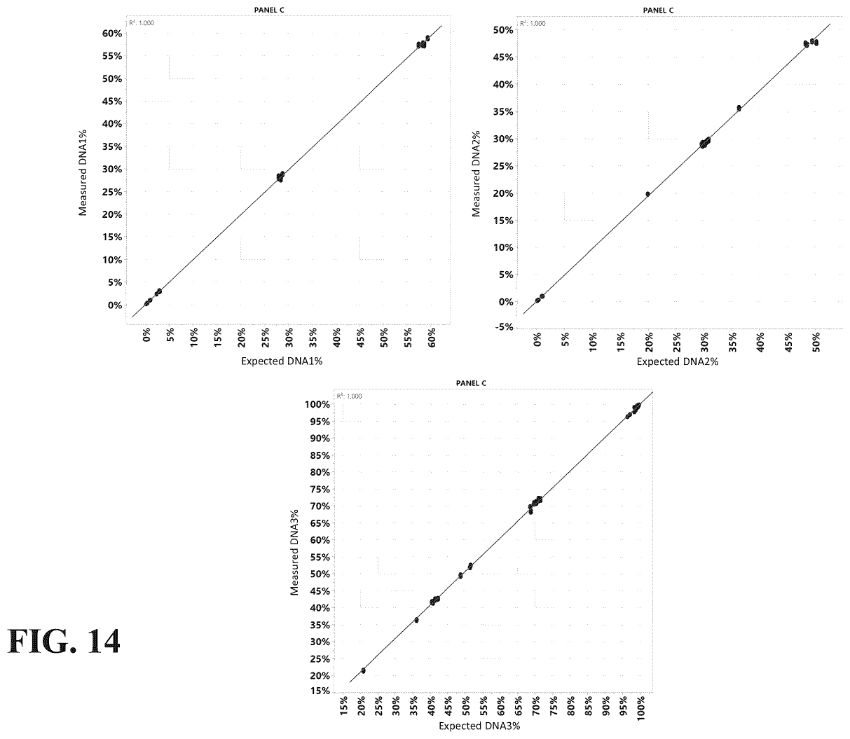

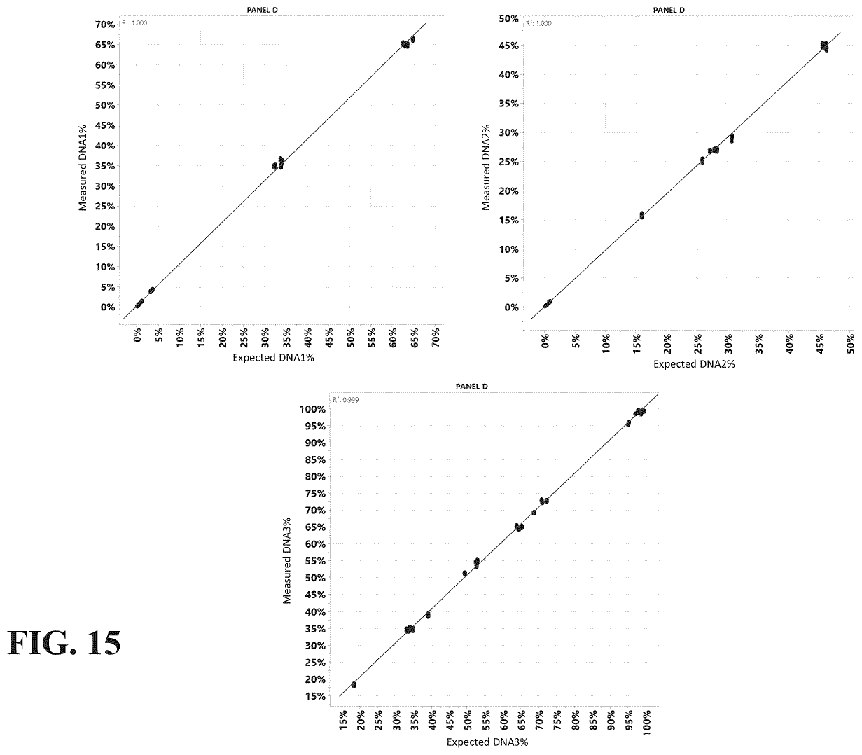

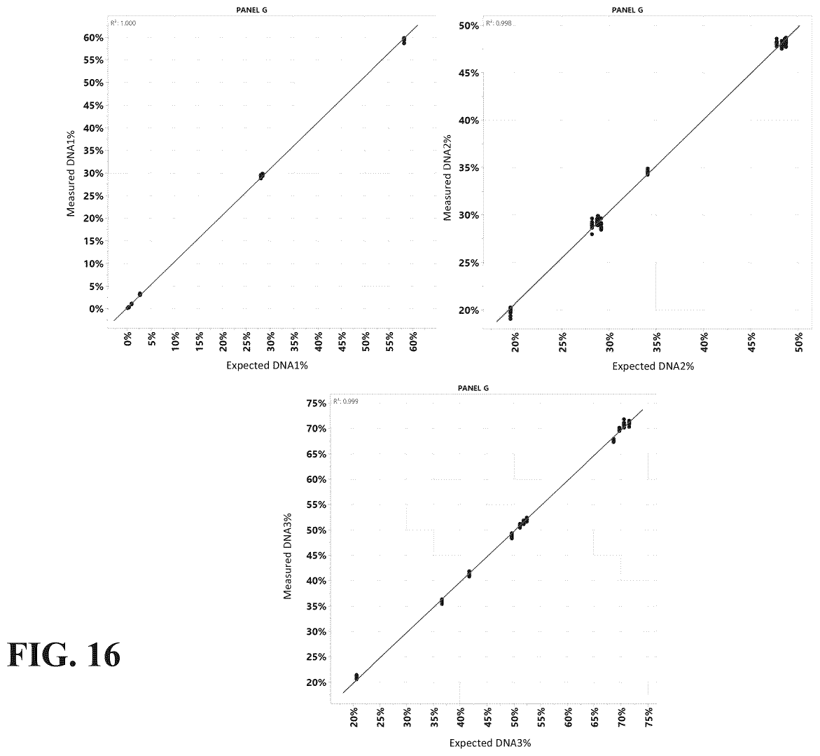

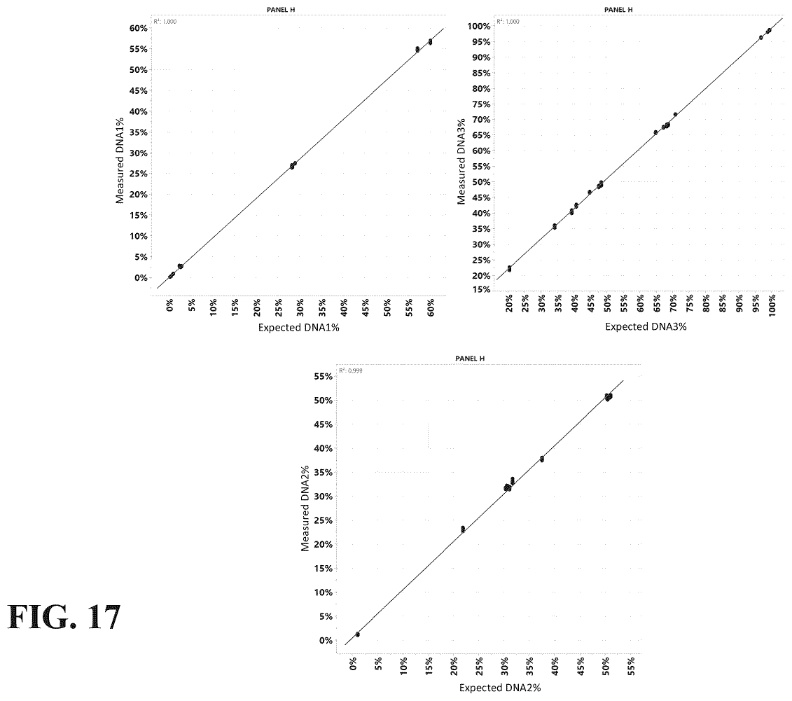

As shown in FIGS. 14-17, the chimerism percentage of allogeneic cell DNA was measured accurately in three genome mixtures containing related (G and H) and unrelated (C and D) genomic DNAs. The results were highly consistent between the replicates and between the high (100-150 ng) and low (8 ng) DNA amount used. Therefore, the method accurately measured levels of chimerism with a high level of linearity in mixtures containing three distinct genomes.

What is claimed is:

1. A method of processing a sample of a subject, said method comprising:
a) receiving a sample obtained from said subject,
wherein said sample comprises a plurality of nucleic acid molecules, and wherein said subject is a recipient of an allograft;
b) isolating said plurality of nucleic acid molecules from said sample;
c) subjecting said isolated said plurality of nucleic acid molecules, or derivative thereof, to one or more amplification reactions using a plurality of primers to generate a plurality of amplicons, and
wherein said plurality of primers target a plurality of independent polymorphisms, and
wherein said plurality of primers are selected without consideration of genotype information from said subject.

2. The method of claim 1, wherein said one or more amplification reactions comprises polymerase chain reaction (PCR).

3. The method of claim 1, further comprising purifying said isolated said plurality of nucleic acid molecules, or a derivative thereof.

4. The method of claim 1, further comprising, after c), sequencing said plurality of amplicons, or derivative thereof.

5. The method of claim 4, wherein said sequencing comprises detecting an optical signal from a probe coupled to an isolated said plurality of nucleic acid molecules, or a derivative thereof.

6. The method of claim 1, further comprising, after c), conducting quantitative polymerase chain reaction (qPCR) of said plurality of amplicons, or derivative thereof.

7. The method of claim 1, further comprising contacting a unique sequence to said plurality of nucleic acid molecules

8. The method of claim 1, wherein said sample is derived from blood.

9. The method of claim 1, further comprising barcoding said plurality of nucleic acids

10. The method of claim 1, wherein said allograft comprises allogenic cells.

11. The method of claim 10, wherein said allogenic cells are selected from the group consisting of hematopoietic stem cells, T cells, B cells, CAR T cells, T reg cells, NK cells, NKT cells, TILs, skeletal muscle stem cells, cardiac stem cells, mesenchymal stem cells, cardiomyocytes, neurons, lymphocytes, macrophages, dendritic cells, and pancreatic islet cells.

12. The method of claim 10, wherein said allogenic cells comprise hematopoietic stem cells.

13. The method of claim 1, wherein said plurality of primers target at least 100 independent polymorphisms.

14. The method of claim 1, wherein said plurality of primers target at least 300 independent polymorphisms.

15. The method of claim 1, further comprising enriching said sample for CD3+, CD15+ or CD33+ cell subtypes.

16. The method of claim 1, wherein said plurality of independent polymorphisms comprise a plurality of single nucleotide polymorphisms.

17. The method of claim 1, wherein said plurality of independent polymorphisms comprise a target population minor allele frequency of >0.4.

18. The method of claim 1, wherein a genomic distance between each independent polymorphism of the plurality of independent polymorphisms is >500 kb.

19. A method comprising:
a) providing cell-free DNA from a sample obtained from a subject who is the recipient of an allograft from a donor,
b) sequencing a panel of single nucleotide polymorphisms (SNPs) from the cell-free DNA, where the panel of SNPs is suitable for differentiating between donor-derived cell-free DNA and recipient-derived cell-free DNA.

20. A system, comprising:
a processor, and
a non-transitory computer readable storage medium encoded with a computer program that causes said processor to:
(a) provide a cell-free sample of a subject, wherein said cell-free sample comprises a plurality of nucleic acid molecules;
(b) subject said plurality of nucleic acid molecules to one or more amplification reactions to generate a plurality of complementary deoxyribonucleic acid (cDNA) molecules or derivative thereof; and
(c) sequence said plurality of cDNA molecules or derivative thereof.
